SEQUENCE LISTING (18 sequences)

SEQ ID NO: 1 - protein, 5 aa, Mus musculus
DNYMH

SEQ ID NO: 2 - protein, 17 aa, Mus musculus
WIDPENGDTEYAPKFRG

SEQ ID NO: 3 - protein, 11 aa, Mus musculus
LIYAGYLAMDY

SEQ ID NO: 4 - protein, 10 aa, Mus musculus
SASSSVTYMH

SEQ ID NO: 5 - protein, 7 aa, Mus musculus
STSNLAS

SEQ ID NO: 6 - protein, 9 aa, Mus musculus
QQRSTYPLT

SEQ ID NO: 7 - protein, 11 aa, Mus sp.
KASQDVGTSVA

SEQ ID NO: 8 - protein, 7 aa, Mus sp.
WTSTRHT

SEQ ID NO: 9 - protein, 8 aa, Mus sp.
QQYSLYRS

SEQ ID NO: 10 - protein, 5 aa, Mus sp.
TYWMS

SEQ ID NO: 11 - protein, 17 aa, Mus sp.
EIHPDSSTINYAPSLKD

SEQ ID NO: 12 - protein, 10 aa, Mus sp.
LYFGFPWFAY

SEQ ID NO: 13 - protein, 15 aa, Artificial Sequence
  source: /note=“Description of Artificial Sequence Synthetic peptide”
KASQSVDYDGDSYLN

SEQ ID NO: 14 - protein, 7 aa, Artificial Sequence
  source: /note=“Description of Artificial Sequence Synthetic peptide”
DASNLVS

SEQ ID NO: 15 - protein, 9 aa, Artificial Sequence
  source: /note=“Description of Artificial Sequence Synthetic peptide”
QQSTEDPWT

SEQ ID NO: 16 - protein, 5 aa, Artificial Sequence
  source: /note=“Description of Artificial Sequence Synthetic peptide”
SYWMN

SEQ ID NO: 17 - protein, 17 aa, Artificial Sequence
  source: /note=“Description of Artificial Sequence Synthetic peptide”
QIWPGDGDTNYNGKFKG

SEQ ID NO: 18 - protein, 15 aa, Artificial Sequence
  source: /note=“Description of Artificial Sequence Synthetic peptide”
RETTTVGRYYYAMDY

(19) United States
(12) Patent Application Publication
(10) Pub. No.: US 2022/0211864 A1
(43) Pub. Date: Jul. 7, 2022

(54) ANTIBODY-DRUG CONJUGATES WITH IMMUNE-MEDIATED THERAPY AGENTS

(71) Applicant: MEDIMMUNE LIMITED, CAMBRIDGE (GB)

(72) Inventors: ROBERT E. HOLLINGSWORTH, GAITHERSBURG, MD (US); JOHN W. HARPER, III, GAITHERSBURG, MD (US); EMIL MICHELOTTI, GAITHERSBURG, MD (US); RAYMOND ROTHSTEIN, GAITHERSBURG, MD (US); JONATHAN RIOS-DORIA, GAITHERSBURG, MD (US)

(21) Appl. No.: 17/524,139

(22) Filed: Nov. 11, 2021

Related U.S. Application Data
(63) Continuation of application No. 16/341,194, filed on Apr. 11, 2019, now abandoned.
(60) Provisional application No. 62/406,630, filed on Oct. 11, 2016.

(51) Int. Cl. A61K 47/68; A61K 9/00; C07K 16/28

(52) U.S. Cl. CPC A61K 47/6851; A61K 47/6803; A61P 35/00; C07K 16/2818; C07K 16/2827; A61K 9/0019

(57) ABSTRACT

Combinations comprising antibody drug conjugates (ADCs) and immunotherapeutic agents (IMTs) are provided. Methods of using such combinations in therapy, e.g. for treatment of cancer, are also provided.

### BACKGROUND

The present invention relates to antibody-drug conjugates and uses thereof, for the treatment or prophylaxis of cancer, in particular use with cancer immunotherapy.

Cancer immunotherapy has revolutionized the way patients with cancer are being treated. Monoclonal antibodies that interfere with immune checkpoints such as CTLA-4 and PD-1 have demonstrated clinical efficacy in multiple tumor types (Wolchok et al., 2013; Callahan and Wolchok, 2013) In addition to these antibodies, several other drugs are being developed that modulate both adaptive and innate immunity (Khalil et al., 2016; Smyth et al., 2016).

Given the resistance that typically occurs following single-agent therapy, it has been hypothesized that combinatorial therapies will improve clinical benefit. Indeed, combination studies with multiple immunotherapies have led to improved response rates and survival (Larkin et al., 2015).

In addition to therapeutics that specifically target the immune system, other agents have been described that have been shown to have some effect on the immune system. It has been shown that some chemotherapies, such as anthracyclines and oxaliplatin, induce immunogenic cell death (ICD) and increase antitumor responses (Rios-Doria et al., 2015; Galluzzi et al., 2012; Tesniere et al., 2010; Obeid et al., 2007), each of which is incorporated herein by reference. ICD is the process by which certain cytotoxic drugs induce apoptosis of tumor cells in a manner which causes the release of immunogenic molecules.

Targeted therapies such as MEK and BRAF inhibitors have also been shown to have some immunomodulatory effects (Liu et al., 2015; Hu-Lieskovan et al., 2015; Vanneman and Dranoff, 2012), each of which is incorporated herein by reference), however it is unclear if these therapeutics elicit ICD or impact the immune system by some other mechanism.

Chemotherapy or targeted therapy requires systemic administration of the drug and thus neither therapy targets tumors per se. The antibody-drug conjugate (ADC) class has been developed to overcome this limitation by conjugating a cyotoxic payload directly to an antibody, which binds tightly and specifically to antigens that are overexpressed on tumor cells. ADCs can provide the localized delivery of cytotoxic payloads to tumors and have been shown to promote intracellular accumulation of the drug within the tumor cells. Such localization provides for relatively high concentrations of drug within the tumor whereas systemic administration of unconjugated (i.e., untargeted) drug to achieve the same tumor concentration may result in unacceptable levels of toxicity to normal cells).

Some ADC payloads have been investigated for their immunomodulatory activity. Müller et al. have demonstrated that ansamitocin P3 and dolastatin induces direct dendritic cell maturation and increased antitumor efficacy when combined with checkpoint blockade (Muller et al., 2014b; Muller et al., 2014a), both of which are incorporated herein by reference). From a mechanistic point of view, combining ADCs with immunotherapy has been contemplated as an approach to increase antitumor responses (Gerber et al., 2016). Indeed, a recent study has demonstrated that T-DM1, an ADC targeting the Her2 receptor, produced in vivo antitumor synergy in mice when combined with anti-CTLA-4 and PD-1, providing initial proof of concept that combining ADC and immunotherapy can produce strong anti-tumor effects (Muller et al., 2015).

The majority of ADCs currently in clinical development are conjugated with microtubule inhibitor payloads: either auristatins or maytansinoids. (Sievers and Senter, 2013), incorporated herein by reference). In this report, two other ADC payloads were investigated for their immunomodulatory activity: PBDs (pyrrolobenzodiazepines) and tubulysins (Hartley, 2011; Li et al., 2016). The mechanism of action of tubulysins is to destabilize tubulin polymers leading to G2/M arrest in mitosis leading to apoptosis, whereas the PBDs form interstrand cross-links in DNA leading to mitotic arrest during S phase and subsequent apoptosis. ADCs with PBD payloads are highlight potent, with in vitro EC50's typically in the low picomolar range (Saunders et al., 2015). Though more potent, the microtubule-destabilizing mechanism of action of tubulysin is similar to that of auristatins and maytansinoids. Despite this, it was shown that such compounds (microtubule-destabilizing) could induce immunogenic effects whereas microtubule-stabilizing compounds could not (Martin et al., 2014). While there is evidence in the literature that certain DNA targeting agents such as radiation and doxorubicin can induce immunogenic cell death, there is no published literature on the ability of PBDs to induce immunogenic cell death and it was not obvious that it would, given that the DNA alkylating agent mitomycin C does not induce ICD (Kroemer et al., 2013; Obeid et al., 2007).

Accordingly, there is need for novel targeted immunomodulatory tumor specific therapies that have potential for treating patients with conditions not adequately met by current approaches

### SUMMARY OF THE INVENTION

The present invention is concerned with antibody-drug conjugates (ADCs) for use in cancer immunotherapy. The invention provides an ADC in combination with an immunotherapy (IMT) agent for use in cancer treatment. For example:

The invention provides an ADC as defined anywhere herein for use in cancer immunotherapy, wherein the use comprises administering to a patient the ADC in combination with an IMT agent. The invention also provides an IMT agent as defined anywhere herein for use in cancer immunotherapy, wherein the use comprises administering to a patient the IMT agent in combination with an ADC.

The invention provides an ADC as defined anywhere herein and an IMT agent as defined anywhere herein for use in cancer immunotherapy, wherein the use comprises administering to a patient the ADC in combination with the IMT agent.

The invention provides an ADC as defined anywhere herein for use in cancer immunotherapy, wherein the use comprises simultaneously, separately or sequentially administering to a patient the ADC in combination with an IMT agent. The invention also provides an IMT agent as defined anywhere herein for use in cancer immunotherapy, wherein the use comprises simultaneously, separately or sequentially administering to a patient the IMT agent in combination with an ADC.

The invention provides an ADC as defined anywhere herein and an IMT agent as defined anywhere herein for use in cancer immunotherapy, wherein the use comprises simultaneously, separately or sequentially administering to a patient the ADC in combination with the IMT agent.

The invention provides a cancer immunotherapy method, the method comprising administering to a patient an ADC as defined anywhere herein and an IMT agent as defined anywhere herein.

The invention provides a cancer immunotherapy method, the method comprising simultaneously, separately or sequentially administering to a patient an ADC as defined anywhere herein and an IMT agent as defined anywhere herein.

In one embodiment, the IMT agent is administered sequentially to the ADC agent. The IMT agent may be administered at least 1 hour, at least 2 hours, at least 4 hours, at least 8 hours, at least 12 hours, at least 24 hours, at least 48 hours, at least 72 hours, at least 96 hours, at least 120 hours after administration of the ADC. The IMT agent may be administered up to 15 days, up to 20 days, up to 25 days or up to 30 days after administration of the ADC.

In one embodiment, the ADC as defined anywhere herein is administered at a lower dosage compared to the dosage required to be therapeutically effective as a monotherapy. In another embodiment, the IMT agent as defined anywhere herein is administered at a lower dosage compared to the dosage required to be therapeutically effective as a monotherapy. In a further embodiment, the ADC as defined anywhere herein and the IMT agent as defined anywhere herein are both administered at lower dosages compared to the respective dosages for the IMT agent or the ADC required to be therapeutically effective as a monotherapy.

In one embodiment, the ADC as defined anywhere herein is administered at a dosage that is at least 90%, at least 80%, at least 70%, at least 60%, at least 50%, at least 40%, at least 30%, at least 20%, at least 10%, at least, 5%, at least 1% lower than the dosage required to be therapeutically effective as a monotherapy. In another embodiment, the IMT agent as defined anywhere herein is administered at a dosage that is at least 90%, at least 80%, at least 70%, at least 60%, at least 50%, at least 40%, at least 30%, at least 20%, at least 10%, at least, 5%, at least 1% lower than the dosage required to be therapeutically effective as a monotherapy. In a further embodiment, the ADC as defined anywhere herein and the IMT agent as defined anywhere herein are both administered at dosages that are at least 90%, at least 80%, at least 70%, at least 60%, at least 50%, at least 40%, at least 30%, at least 20%, at least 10%, at least, 5%, at least 1% lower than the respective dosages for the IMT agent or the ADC required to be therapeutically effective as a monotherapy. Suitable models for observing tumor growth are well known to the person skilled in the art and may vary depending on the indication being investigated.

In one embodiment, the drug of the ADC as defined anywhere herein is pyrrolobenzodiazepine (PBD). In another embodiment, the drug of the ADC as defined anywhere herein is tubulysin.

In one embodiment, the ADC as defined anywhere herein is administered intravenously. In one embodiment, the ADC as defined anywhere herein is administered intratumorally. In one embodiment, the IMT agent as defined anywhere herein is administered intravenously. In one embodiment, the IMT agent as defined anywhere herein is administered intraperitoneally. In one embodiment, the IMT agent as defined anywhere herein is administered intratumorally.

In one embodiment, the IMT agent as defined anywhere herein is a checkpoint inhibitor. In another embodiment, the IMT agent as defined anywhere herein is an agonist of the tumor necrosis factor (TNF) receptor superfamily. In one embodiment, the IMT agent as defined anywhere herein is selected from the group consisting of: a programmed cell death protein-1 (PD-1) inhibitor, a programmed death-ligand-1 (PD-L1) inhibitor, an OX40 agonist, and a glucocorticoid-induced TNFR-related protein (GITR) agonist. In a further embodiment, the IMT agent as defined anywhere herein is selected from the group consisting of: an anti-PD-1 antibody, an anti-PD-L1 antibody, an anti-OX40 antibody, OX40 ligand fusion protein and a GITRL fusion protein.

In one embodiment, the antibody as defined anywhere above is an antibody recognizing a tumor-associated antigen or antigen-binding fragment thereof. Exemplary tumour-associated antigens against which antibodies may be generated for use in embodiments of the present invention are listed below.

Exemplary antibodies against tumor-associated antigens or antigen-binding fragments thereof for use in embodiments of the present invention are also listed below.

### Tumor-Associated Antigens and Cognate Antibodies

### (1) BMPRIB (Bone Morphogenetic Protein Receptor-Type IB)

Nucleotide:
Genbank accession no. NM_001203
Genbank version no. NM_001203.2 GI:169790809
Genbank record update date: Sep. 23, 2012 02:06 PM

Polypeptide:
Genbank accession no. NP_001194
Genbank version no. NP_001194.1 GI:4502431
Genbank record update date: Sep. 23, 2012 02:06 PM

### CROSS-REFERENCES

ten Dijke,P., et al Science 264 (5155): 101-104 (1994), Oncogene 14 (11):1377-1382 (1997)); WO2004/063362 (claim 2); WO2003/042661 (claim 12); US2003/134790-A1 (Page 38-39); WO2002/102235 (claim 13; Page 296); WO2003/055443 (Page 91-92); WO2002/99122 (Example 2; Page 528-530); WO2003/029421 (claim 6); WO2003/024392 (claim 2; FIG. 112); WO2002/98358 (claim 1; Page 183); WO2002/54940 (Page 100-101); WO2002/59377 (Page 349-350); WO2002/30268 (claim 27; Page 376); WO2001/48204 (Example; FIG. 4); NP_001194 bone morphogenetic protein receptor, type IB/pid=NP_001194.1; MIM:603248; AY065994.

### (2) E16 (LAT1, SLC7A5)

Nucleotide:
Genbank accession no. NM_003486
Genbank version no. NM_003486.5 GI:71979931
Genbank record update date: Jun. 27, 2012 12:06 PM

Polypeptide:
Genbank accession no. NP_003477
Genbank version no. NP_003477.4 GI:71979932
Genbank record update date: Jun. 27, 2012 12:06 PM

### CROSS REFERENCES

Biochem. Biophys. Res. Commun. 255 (2), 283-288 (1999), Nature 395 (6699):288-291 (1998), Gaugitsch, H. W., et al (1992) J. Biol. Chem. 267 (16):11267-11273); WO2004/048938 (Example 2); WO2004/032842 (Example IV); WO2003/042661 (claim 12); WO2003/016475 (claim 1); WO2002/78524 (Example 2); WO2002/99074 (claim 19; Page 127-129); WO2002/86443 (claim 27; Pages 222, 393); WO2003/003906 (claim 10; Page 293); WO2002/64798 (claim 33; Page 93-95); WO2000/14228 (claim 5; Page 133-136); US2003/224454 (FIG. 3); WO2003/025138 (claim 12; Page 150); NP_003477 solute carrier family 7 (cationic amino acid transporter, y+system), member 5/pid=NP_003477.3—Homo sapiens; MIM:600182; NM_015923.

### (3) STEAPI (Six Transmembrane Epithelial Antigen of Prostate)

Nucleotide:
Genbank accession no. NM_012449
Genbank version no. NM_012449.2 GI:22027487
Genbank record update date: Sep. 9, 2012 02:57 PM

Polypeptide:
Genbank accession no. NP_036581
Genbank version no. NP_036581.1 GI:9558759
Genbank record update date: Sep. 9, 2012 02:57 PM

### CROSS REFERENCES

Cancer Res. 61 (15), 5857-5860 (2001), Hubert, R. S., et al (1999) Proc. Natl. Acad. Sci. U.S.A. 96 (25):14523-14528); WO2004/065577 (claim 6); WO2004/027049 (FIG. 1L); EP1394274 (Example 11); WO2004/016225 (claim 2); WO2003/042661 (claim 12); US2003/157089 (Example 5); US2003/185830 (Example 5); US2003/064397 (FIG. 2); WO2002/89747 (Example 5; Page 618-619); WO2003/022995 (Example 9; FIG. 13A, Example 53; Page 173, Example 2; FIG. 2A); six transmembrane epithelial antigen of the prostate; MIM:604415.

### (4) 0772P (CA125, MUC16)

Nucleotide:
Genbank accession no. AF361486
Genbank version no. AF361486.3 GI:34501466
Genbank record update date: Mar. 11, 2010 07:56 AM

Polypeptide:
Genbank accession no. AAK74120
Genbank version no. AAK74120.3 GI:34501467
Genbank record update date: Mar. 11, 2010 07:56 AM

### CROSS REFERENCES

J. Biol. Chem. 276 (29):27371-27375 (2001)); WO2004/045553 (claim 14); WO2002/92836 (claim 6; FIG. 12); WO2002/83866 (claim 15; Page 116-121); US2003/124140 (Example 16); GI:34501467.

### (5) MPF (MPF, MSLN, SMR, Megakaryocyte Potentiating Factor, Mesothelin)

Nucleotide:
Genbank accession no. NM_005823
Genbank version no. NM_005823.5 GI:293651528
Genbank record update date: Sep. 2, 2012 01:47 PM

Polypeptide:
Genbank accession no. NP_005814
Genbank version no. NP_005814.2 GI:53988378
Genbank record update date: Sep. 2, 2012 01:47 PM

### CROSS REFERENCES

Yamaguchi, N., et al Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J. Biol. Chem. 270 (37):21984-21990 (1995)); WO2003/101283 (claim 14); (WO2002/102235 (claim 13; Page 287-288); WO2002/101075 (claim 4; Page 308-309); WO2002/71928 (Page 320-321); WO94/10312 (Page 52-57); IM:601051.

### (6) Napi3b (NAPI-3B, NPTIIb, SLC34A2, Solute Carrier Family 34 (Sodium Phosphate), Member 2, Type II Sodium-Dependent Phosphate Transporter 3b)

Nucleotide:
Genbank accession no. NM_006424
Genbank version no. NM_006424.2 GI:110611905
Genbank record update date: Jul. 22, 2012 03:39 PM

Polypeptide:
Genbank accession no. NP_006415
Genbank version no. NP_006415.2 GI:110611906
Genbank record update date: Jul. 22, 2012 03:39 PM

### CROSS REFERENCES

J. Biol. Chem. 277 (22):19665-19672 (2002), Genomics 62 (2):281-284 (1999), Feild, J. A., et al (1999) Biochem. Biophys. Res. Commun. 258 (3):578-582); WO2004/022778 (claim 2); EP1394274 (Example 11); WO2002/102235 (claim 13; Page 326); EP0875569 (claim 1; Page 17-19); WO2001/57188 (claim 20; Page 329); WO2004/032842 (Example IV); WO2001/75177 (claim 24; Page 139-140); MIM:604217.

### (7) Sema 5b (FLJ10372, KIAA1445, Mm.42015, SEMA5B, SEMAG, Semaphorin 5b Hlog, Sema Domain, Seven Thrombospondin Repeats (Type 1 and Type 1-Like), Transmembrane Domain (TM) and Short Cytoplasmic Domain, (Semaphorin) 5B)

Nucleotide:
Genbank accession no. AB040878
Genbank version no. AB040878.1 GI:7959148
Genbank record update date: Aug. 2, 2006 05:40 PM

Polypeptide:
Genbank accession no. BAA95969
Genbank version no. BAA95969.1 GI:7959149
Genbank record update date: Aug. 2, 2006 05:40 PM

### CROSS REFERENCES

Nagase T., et al (2000) DNA Res. 7 (2):143-150); WO2004/000997 (claim 1); WO2003/003984 (claim 1); WO2002/06339 (claim 1; Page 50); WO2001/88133 (claim 1; Page 41-43, 48-58); WO2003/054152 (claim 20); WO2003/101400 (claim 11); Accession: 30 Q9P283; Genew; HGNC:10737.

(8) PSCA hlg (2700050C12Rik, C530008O16Rik, RIKEN cDNA 2700050C12, RIKEN cDNA 2700050C12 gene)

Nucleotide:
Genbank accession no. AY358628
Genbank version no. AY358628.1 GI:37182377
Genbank record update date: Dec. 1, 2009 04:15 AM

Polypeptide:
Genbank accession no. AAQ88991
Genbank version no. AAQ88991.1 GI:37182378
Genbank record update date: Dec. 1, 2009 04:15 AM

### CROSS REFERENCES

Ross et al (2002) Cancer Res. 62:2546-2553; US2003/129192 (claim 2); US2004/044180 (claim 12); US2004/044179 (claim 11); US2003/096961 (claim 11); US2003/232056 (Example 5); WO2003/105758 16 (claim 12); US2003/206918 (Example 5); EP1347046 (claim 1); WO2003/025148 (claim 20); GI:37182378.

(9) ETBR (Endothelin type B receptor)

Nucleotide:
Genbank accession no. AY275463
Genbank version no. AY275463.1 GI:30526094
Genbank record update date: Mar. 11, 2010 02:26 AM

Polypeptide:
Genbank accession no. AAP32295
Genbank version no. AAP32295.1 GI:30526095
Genbank record update date: Mar. 11, 2010 02:26 AM

### CROSS REFERENCES

Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, s1-S4, 1992; Tsutsumi M., et al Gene 228, 43-49, 1999; Strausberg R. L., et al Proc. Natl. Acad. Sci. U.S.A. 99, 16899-16903, 2002; Bourgeois C., et al J. Clin. Endocrinol. Metab. 82, 3116-3123, 1997; Okamoto Y., et al Biol. Chem. 272, 21589-21596, 1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P. J., et al Hum. Genet. 103, 145-148, 1998; Fuchs S., et al Mol. Med. 7, 115-124, 2001; Pingault V., et al (2002) Hum. Genet. 111, 198-206; WO2004/045516 (claim 1); WO2004/048938 (Example 2); WO2004/040000 (claim 151); WO2003/087768 (claim 1); WO2003/016475 (claim 1); WO2003/016475 (claim 1); WO2002/61087 (FIG. 1); WO2003/016494 (FIG. 6); WO2003/025138 (claim 12; Page 144); WO2001/98351 (claim 1; Page 124-125); EP0522868 (claim 8; FIG. 2); WO2001/77172 (claim 1; Page 297-299); US2003/109676; U.S. Pat. No. 6,518,404 (FIG. 3); U.S. Pat. No. 5,773,223 (claim 1a; Col 31-34); WO2004/001004.

### (10) MSG783 (RNF124, Hypothetical Protein FLJ20315)

Nucleotide:
Genbank accession no. NM_017763
Genbank version no. NM_017763.4 GI:167830482
Genbank record update date: Jul. 22, 2012 12:34 AM

Polypeptide:
Genbank accession no. NP_060233
Genbank version no. NP_060233.3 GI:56711322
Genbank record update date: Jul. 22, 2012 12:34 AM

### CROSS REFERENCES

WO2003/104275 (claim 1); WO2004/046342 (Example 2); WO2003/042661 (claim 12); WO2003/083074 (claim 14; Page 61); WO2003/018621 (claim 1); WO2003/024392 (claim 2; FIG. 93); WO2001/66689 (Example 6); LocusID:54894.

### (11) STEAP2 (HGNC_8639, IPCA-1, PCANAP1, STAMP1, STEAP2, STMP, Prostate Cancer Associated Gene 1, Prostate Cancer Associated Protein 1, Six Transmembrane Epithelial Antigen of Prostate 2, Six Transmembrane Prostate Protein)

Nucleotide:
Genbank accession no. AF455138
Genbank version no. AF455138.1 GI:22655487
Genbank record update date: Mar. 11, 2010 01:54 AM

Polypeptide:
Genbank accession no. AAN04080
Genbank version no. AAN04080.1 GI:22655488
Genbank record update date: Mar. 11, 2010 01:54 AM

### CROSS REFERENCES

Lab. Invest. 82 (11):1573-1582 (2002)); WO2003/087306; US2003/064397 (claim 1; FIG. 1); WO2002/72596 (claim 13; Page 54-55); WO2001/72962 (claim 1; FIG. 4B); WO2003/104270 (claim 11); WO2003/104270 (claim 16); US2004/005598 (claim 22); WO2003/042661 (claim 12); US2003/060612 (claim 12; FIG. 10); WO2002/26822 (claim 23; FIG. 2); WO2002/16429 (claim 12; FIG. 10); GI:22655488.

### (12) TrpM4 (BR22450, FLJ20041, TRPM4, TRPM4B, Transient Receptor Potential Cation Channel, Subfamily M, Member 4)

Nucleotide:
Genbank accession no. NM_017636
Genbank version no. NM_017636.3 GI:304766649
Genbank record update date: Jun. 29, 2012 11:27 AM

Polypeptide:
Genbank accession no. NP_060106
Genbank version no. NP_060106.2 GI:21314671
Genbank record update date: Jun. 29, 2012 11:27 AM

### CROSS REFERENCES

Xu, X. Z., et al Proc. Natl. Acad. Sci. U.S.A. 98 (19):10692-10697 (2001), Cell 109 (3):397-407 (2002), J. Biol. Chem. 278 (33):30813-30820 (2003)); US2003/143557 (claim 4); WO2000/40614 (claim 14; Page 100-103); WO2002/10382 (claim 1; FIG. 9A); WO2003/042661 (claim 12); WO2002/30268 (claim 27; Page 391); US2003/219806 (claim 4); WO2001/62794 (claim 14; FIG. 1A-D); MIM:606936.

### (13) CRIPTO (CR, CR1, CRGF, CRIPTO, TDGF1, Teratocarcinoma-Derived Growth Factor)

Nucleotide:
Genbank accession no. NM_003212
Genbank version no. NM_003212.3 GI:292494881
Genbank record update date: Sep. 23, 2012 02:27 PM

Polypeptide:
Genbank accession no. NP_003203
Genbank version no. NP_003203.1 GI:4507425
Genbank record update date: Sep. 23, 2012 02:27 PM

### CROSS REFERENCES

Ciccodicola, A., et al EMBO J. 8 (7):1987-1991 (1989), Am. J. Hum. Genet. 49 (3):555-565 (1991)); US2003/224411 (claim 1); WO2003/083041 (Example 1); WO2003/034984 (claim 12); WO2002/88170 (claim 2; Page 52-53); WO2003/024392 (claim 2; FIG. 58); WO2002/16413 (claim 1; Page 94-95, 105); WO2002/22808 (claim 2; FIG. 1); U.S. Pat. No. 5,854,399 (Example 2; Col 17-18); U.S. Pat. No. 5,792,616 (FIG. 2); MIM:187395.

### (14) CD21 (CR2 (Complement Receptor 2) or C3DR (C3d/Epstein Barr Virus Receptor) or Hs.73792)

Nucleotide:
Genbank accession no M26004
Genbank version no. M26004.1 GI:181939
Genbank record update date: Jun. 23, 2010 08:47 AM

Polypeptide:
Genbank accession no. AAA35786
Genbank version no. AAA35786.1 GI:181940
Genbank record update date: Jun. 23, 2010 08:47 AM

### CROSS REFERENCES

Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004/045520 (Example 4); US2004/005538 (Example 1); WO2003/062401 (claim 9); WO2004/045520 (Example 4); WO91/02536 (FIGS. 9.1-9.9); WO2004/020595 (claim 1); Accession: P20023; Q13866; Q14212; EMBL; M26004; AAA35786.1.

### (15) CD79b (CD79B, CD79p, IGb (Immunoglobulin-Associated Beta), B29)

Nucleotide:
Genbank accession no NM_000626
Genbank version no. NM_000626.2 GI:90193589
Genbank record update date: Jun. 26, 2012 01:53 PM

Polypeptide:
Genbank accession no. NP_000617
Genbank version no. NP_000617.1 GI:11038674
Genbank record update date: Jun. 26, 2012 01:53 PM

### CROSS REFERENCES

Proc. Natl. Acad. Sci. U.S.A. (2003) 100 (7):4126-4131, Blood (2002) 100 (9):3068-3076, Muller et al (1992) Eur. J. Immunol. 22 (6):1621-1625); WO2004/016225 (claim 2, FIG. 140); WO2003/087768, US2004/101874 (claim 1, page 102); WO2003/062401 (claim 9); WO2002/78524 (Example 2); US2002/150573 (claim 5, page 15); U.S. Pat. No. 5,644,033; WO2003/048202 (claim 1, pages 306 and 309); WO 99/58658, U.S. Pat. No. 6,534,482 (claim 13, FIG. 17A/B); WO2000/55351 (claim 11, pages 1145-1146); MIM:147245 (16) FcRH2 (IFGP4, IRTA4, SPAPIA (SH2 domain containing phosphatase anchor protein 1a), SPAP1B, SPAP1C)

Nucleotide:
Genbank accession no NM_030764
Genbank version no. NM_030764.3 GI:227430280
Genbank record update date: Jun. 30, 2012 12:30 AM

Polypeptide:
Genbank accession no. NP_110391
Genbank version no. NP_110391.2 GI:19923629
Genbank record update date: Jun. 30, 2012 12:30 AM

### CROSS REFERENCES

AY358130); Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004/016225 (claim 2); WO2003/077836; WO2001/38490 (claim 5; FIG. 18D-1-18D-2); WO2003/097803 (claim 12); WO2003/089624 (claim 25); MIM:606509.

### (17) HER2 (ErbB2)

Nucleotide:
Genbank accession no M11730
Genbank version no. M11730.1 GI:183986
Genbank record update date: Jun. 23, 2010 08:47 AM

Polypeptide:
Genbank accession no. AAA75493
Genbank version no. AAA75493.1 GI:306840
Genbank record update date: Jun. 23, 2010 08:47 AM

### CROSS REFERENCES

Coussens L., et al Science (1985) 230(4730):1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-880, 2004; Kuhns J. J., et al J. Biol. Chem. 274, 36422-36427, 1999; Cho H.-S., et al Nature 421, 756-760, 2003; Ehsani A., et al (1993) Genomics 15, 426-429; WO2004/048938 (Example 2); WO2004/027049 (FIG. 11); WO2004/009622; WO2003/081210; WO2003/089904 (claim 9); WO2003/016475 (claim 1); US2003/118592; WO2003/008537 (claim 1); WO2003/055439 (claim 29; FIG. 1A-B); WO2003/025228 (claim 37; FIG. 5C); WO2002/22636 (Example 13; Page 95-107); WO2002/12341 (claim 68; FIG. 7); WO2002/13847 (Page 71-74); WO2002/14503 (Page 114-117); WO2001/53463 (claim 2; Page 41-46); WO2001/41787 (Page 15); WO2000/44899 (claim 52; FIG. 7); WO2000/20579 (claim 3; FIG. 2); U.S. Pat. No. 5,869,445 (claim 3; Col 31-38); WO9630514 (claim 2; Page 56-61); EP1439393 (claim 7); WO2004/043361 (claim 7); WO2004/022709; WO2001/00244 (Example 3; FIG. 4); Accession: P04626; EMBL; M11767; AAA35808.1. EMBL; M11761; AAA35808.1

Antibodies:
Abbott: US20110177095
   for example, an antibody comprising CDRs having overall at least 80% sequence identity to CDRs having amino acid sequences of SEQ ID NO:3 (CDR-H1), SEQ ID NO:4 (CDR-H2), SEQ ID NO:5 (CDR-H3), SEQ ID NO:104 and/or SEQ ID NO:6 (CDR-L1), SEQ ID NO:7 (CDR-L2), and SEQ ID NO:8 (CDR-L3), wherein the anti-HER2 antibody or anti-HER2 binding fragment has reduced immunogenicity as compared to an antibody having a VH of SEQ ID NO:1 and a VL of SEQ ID NO:2.
Biogen: US20100119511
   for example, ATCC accession numbers: PTA-10355, PTA-10356, PTA-10357, PTA 10358
   for example, a purified antibody molecule that binds to HER2 comprising a all six CDR's from an antibody selected from the group consisting of BIIB71F10 (SEQ ID NOs:11, 13), BIIB69A09 (SEQ ID NOs:15, 17); BIIB67F10 (SEQ ID NOs:19, 21); BIIB67F11 (SEQ ID NOs:23, 25), BIIB66A12 (SEQ ID NOs:27, 29), BIIB66C01 (SEQ ID NOs:31, 33), BIIB65C10 (SEQ ID NOs:35, 37), BIIB65H09 (SEQ ID NOs:39, 41) and B116651303 (SEQ ID NOs:43, 45), or CDRs which are identical or which have no more than two alterations from said CDRs.
Herceptin (Genentech)—U.S. Pat. No. 6,054,297; ATCC accession no. CRL-10463 (Genentech)
Pertuzumab (Genentech) US20110117097
   for example, see SEQ IDs No. 15&16, SEQ IDs No. 17&18, SEQ IDs No. 23&24 & ATCC accession numbers HB-12215, HB-12216, CRL 10463, HB-12697.
US20090285837
US20090202546
   for example, ATCC accession numbers: HB-12215, HB-12216, CRL 10463, HB-12698.
US20060088523
   for example, ATCC accession numbers: HB-12215, HB-12216
   for example, an antibody comprising the variable light and variable heavy amino acid sequences in SEQ ID Nos. 3 and 4, respectively.
   for example, an antibody comprising a light chain amino acid sequence selected from SEQ ID No. 15 and 23, and a heavy chain amino acid sequence selected from SEQ ID No. 16 and 24
US20060018899
   for example, ATCC accession numbers: (7C2) HB-12215, (7F3) HB-12216, (4D5) CRL-10463, (2C4) HB-12697.
   for example, an antibody comprising the amino acid sequence in SEQ ID No. 23, or a deamidated and/or oxidized variant thereof.
US2011/0159014
   for example, an antibody having a light chain variable domain comprising the hypervariable regions of SEQ ID NO: 1.
   For example, an antibody having a heavy chain variable domain comprising the hypervariable regions of SEQ ID NO: 2.
US20090187007
Glycotope: TrasGEX antibody http://www.glycotope.com/pipeline
   for example, see International Joint Cancer Institute and Changhai Hospital Cancer Cent: HMTI-Fc Ab—Gao J., et al BMB Rep. 2009 Oct. 31; 42(10):636-41.
Symphogen: US20110217305
Union Stem Cell &Gene Engineering, China—Liu H Q., et al Xi Bao Yu Fen Zi Mian Yi Xue Za Zhi. 2010 May; 26(5):456-8.

### (18) NCA (CEACAM6)

Nucleotide:
Genbank accession no M18728
Genbank version no. M18728.1 GI:189084
Genbank record update date: Jun. 23, 2010 08:48 AM

Polypeptide:
Genbank accession no. AAA59907
Genbank version no. AAA59907.1 GI:189085
Genbank record update date: Jun. 23, 2010 08:48 AM

### CROSS REFERENCES

Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99:16899-16903, 2002; WO2004/063709; EP1439393 (claim 7); WO2004/044178 (Example 4); WO2004/031238; WO2003/042661 (claim 12); WO2002/78524 (Example 2); WO2002/86443 (claim 27; Page 427); WO2002/60317 (claim 2); Accession: P40199; Q14920; EMBL; M29541; AAA59915.1. EMBL; M18728.

### (19) MDP (DPEP1)

Nucleotide:
Genbank accession no BC017023
Genbank version no. BC017023.1 GI:16877538
Genbank record update date: Mar. 6, 2012 01:00 PM

Polypeptide:
Genbank accession no. AAH17023
Genbank version no. AAH17023.1 GI:16877539
Genbank record update date: Mar. 6, 2012 01:00 PM

### CROSS REFERENCES

Proc. Natl. Acad. Sci. U.S.A. 99 (26):16899-16903 (2002)); WO2003/016475 (claim 1); WO2002/64798 (claim 33; Page 85-87); JP05003790 (FIG. 6-8); WO99/46284 (FIG. 9); MIM:179780.

### (20) IL20R-alpha (IL20Ra, ZCYTOR7)

Nucleotide:
Genbank accession no AF184971
Genbank version no. AF184971.1 GI:6013324
Genbank record update date: Mar. 10, 2010 10:00 PM

Polypeptide:
Genbank accession no. AAF01320
Genbank version no. AAF01320.1 GI:6013325
Genbank record update date: Mar. 10, 2010 10:00 PM

### CROSS REFERENCES

Clark H. F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) Biochemistry 42:12617-12624; Sheikh F., et al (2004) J. Immunol. 172, 2006-2010; EP1394274 (Example 11); US2004/005320 (Example 5); WO2003/029262 (Page 74-75); WO2003/002717 (claim 2; Page 63); WO2002/22153 (Page 45-47); US2002/042366 (Page 20-21); WO2001/46261 (Page 57-59); WO2001/46232 (Page 63-65); WO98/37193 (claim 1; Page 55-59); Accession: Q9UHF4; Q6UWA9; Q96SH8; EMBL; AF184971; AAF01320.1.

### (21) Brevican (BCAN, BEHAB)

Nucleotide:
Genbank accession no AF229053
Genbank version no. AF229053.1 GI:10798902
Genbank record update date: Mar. 11, 2010 12:58 AM

Polypeptide:
Genbank accession no. AAG23135
Genbank version no. AAG23135.1 GI:10798903
Genbank record update date: Mar. 11, 2010 12:58 AM

### CROSS REFERENCES

Gary S.C., et al Gene 256, 139-147, 2000; Clark H.F., et al Genome Res. 13, 2265-2270, 2003; Strausberg R. L., et al Proc. Natl. Acad. Sci. U.S.A. 99, 16899-16903, 2002; US2003/186372 (claim 11); US2003/186373 (claim 11); US2003/119131 (claim 1; FIG. 52); US2003/119122 (claim 1; FIG. 52); US2003/119126 (claim 1); US2003/119121 (claim 1; FIG. 52); US2003/119129 (claim 1); US2003/119130 (claim 1); US2003/119128 (claim 1; FIG. 52); US2003/119125 (claim 1); WO2003/016475 (claim 1); WO2002/02634 (claim 1) (22) EphB2R (DRT, ERK, Hek5, EPHT3, Tyro5)

Nucleotide:
Genbank accession no NM_004442
Genbank version no. NM_004442.6 GI:111118979
Genbank record update date: Sep. 8, 2012 04:43 PM

Polypeptide:
Genbank accession no. NP_004433
Genbank version no. NP_004433.2 GI:21396504
Genbank record update date: Sep. 8, 2012 04:43 PM

### CROSS REFERENCES

Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (claim 12); WO200053216 (claim 1; Page 41); WO2004065576 (claim 1); WO2004020583 (claim 9); WO2003004529 (Page 128-132); WO200053216 (claim 1; Page 42); MIM:600997.

### (23) ASLG659 (B7h)

Nucleotide:
Genbank accession no. AX092328
Genbank version no. AX092328.1 GI:13444478
Genbank record update date: Jan. 26, 2011 07:37 AM

### CROSS REFERENCES

US2004/0101899 (claim 2); WO2003104399 (claim 11); WO2004000221 (FIG. 3); US2003/165504 (claim 1); US2003/124140 (Example 2); US2003/065143 (FIG. 60); WO2002/102235 (claim 13; Page 299); US2003/091580 (Example 2); WO2002/10187 (claim 6; FIG. 10); WO2001/94641 (claim 12; FIG. 7b); WO2002/02624 (claim 13; FIG. 1A-16); US2002/034749 (claim 54; Page 45-46); WO2002/06317 (Example 2; Page 320-321, claim 34; Page 321-322); WO2002/71928 (Page 468-469); WO2002/02587 (Example 1; FIG. 1); WO2001/40269 (Example 3; Pages 190-192); WO2000/36107 (Example 2; Page 205-207); WO2004/053079 (claim 12); WO2003/004989 (claim 1); WO2002/71928 (Page 233-234, 452-453); WO 01/16318.

(24) PSCA (Prostate stem cell antigen precursor)

Nucleotide:
Genbank accession no AJ297436
Genbank version no. AJ297436.1 GI:9367211
Genbank record update date: Feb. 1, 2011 11:25 AM

Polypeptide:
Genbank accession no. CAB97347
Genbank version no. CAB97347.1 GI:9367212
Genbank record update date: Feb. 1, 2011 11:25 AM

### CROSS REFERENCES

Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004/022709; EP1394274 (Example 11); U52004/018553 (claim 17); WO2003/008537 (claim 1); WO2002/81646 (claim 1; Page 164); WO2003/003906 (claim 10; Page 288); WO2001/40309 (Example 1; FIG. 17); US2001/055751 (Example 1; FIG. 1b); WO2000/32752 (claim 18; FIG. 1); WO98/51805 (claim 17; Page 97); WO98/51824 (claim 10; Page 94); WO98/40403 (claim 2; FIG. 1B); Accession: 043653; EMBL; AF043498; AAC39607.1 (25) GEDA

Nucleotide:
Genbank accession no AY260763
Genbank version no. AY260763.1 GI:30102448
Genbank record update date: Mar. 11, 2010 02:24 AM

Polypeptide:
Genbank accession no. AAP14954
Genbank version no. AAP14954.1 GI:30102449
Genbank record update date: Mar. 11, 2010 02:24 AM

### CROSS REFERENCES

AP14954 lipoma HMGIC fusion-partnerlike protein/pid=AAP14954.1—Homo sapiens (human); WO2003/054152 (claim 20); WO2003/000842 (claim 1); WO2003/023013 (Example 3, claim 20); US2003/194704 (claim 45); GI:30102449.

### (26) BAFF-R (B Cell-Activating Factor Receptor, BLyS Receptor 3, BR3)

Nucleotide:
Genbank accession no AF116456
Genbank version no. AF116456.1 GI:4585274
Genbank record update date: Mar. 10, 2010 09:44 PM

Polypeptide:
Genbank accession no. AAD25356
Genbank version no. AAD25356.1 GI:4585275
Genbank record update date: Mar. 10, 2010 09:44 PM

### CROSS REFERENCES

BAFF receptor/pid=NP_443177.1—Homo sapiens: Thompson, J. S., et al Science 293 (5537), 2108-2111 (2001); WO2004/058309; WO2004/011611; WO2003/045422 (Example; Page 32-33); WO2003/014294 (claim 35; FIG. 6B); WO2003/035846 (claim 70; Page 615-616); WO2002/94852 (Col 136-137); WO2002/38766 (claim 3; Page 133); WO2002/24909 (Example 3; FIG. 3); MIM:606269; NP_443177.1; NM_052945_1; AF132600 (27) CD22 (B-cell receptor CD22-B isoform, BL-CAM, Lyb-8, Lyb8, SIGLEC-2, FLJ22814)

Nucleotide:
Genbank accession no AK026467
Genbank version no. AK026467.1 GI:10439337
Genbank record update date: Sep. 11, 2006 11:24 PM

Polypeptide:
Genbank accession no. BAB15489
Genbank version no. BAB15489.1 GI:10439338
Genbank record update date: Sep. 11, 2006 11:24 PM

### CROSS REFERENCES

Wilson et al (1991) J. Exp. Med. 173:137-146; WO2003/072036 (claim 1; FIG. 1); IM:107266; NP_001762.1; NM_001771_1.

(27a) CD22 (CD22 molecule)

Nucleotide:
Genbank accession no X52785
Genbank version no. X52785.1 GI:29778
Genbank record update date: Feb. 2, 2011 10:09 AM

Polypeptide:
Genbank accession no. CAA36988
Genbank version no. CAA36988.1 GI:29779
Genbank record update date: Feb. 2, 2011 10:09 AM

### CROSS REFERENCES

Stamenkovic I. et al., Nature 345 (6270), 74-77 (1990)??

Other Information:
Official Symbol: CD22
Other Aliases: SIGLEC-2, SIGLEC2
Other Designations: B-cell receptor CD22; B-lymphocyte cell adhesion molecule; BL-CAM; CD22 antigen; T-cell surface antigen Leu-14; sialic acid binding Ig-like lectin 2; sialic acid-binding Ig-like lectin 2

### Antibodies:

G5/44 (Inotuzumab): DiJoseph J F.,et al Cancer Immunol Immunother. 2005 January; 54(1):11-24.
Epratuzumab—Goldenberg D M., et al Expert Rev Anticancer Ther. 6(10): 1341-53, 2006.
   (28) CD79a (CD79A, CD79alpha), Immunoglobulin-Associated Alpha, a B Cell-Specific Protein that Covalently Interacts with Ig Beta (CD79B) and Forms a Complex on the Surface with Ig M Molecules, Transduces a Signal Involved in B-Cell Differentiation), pl: 4.84, MW: 25028 TM: 2 [P] Gene Chromosome: 19q13.2).

Nucleotide:
Genbank accession no NM_001783
Genbank version no. NM_001783.3 GI:90193587
Genbank record update date: Jun. 26, 2012 01:48 PM

Polypeptide:
Genbank accession no. NP_001774
Genbank version no. NP_001774.1 GI:4502685
Genbank record update date: Jun. 26, 2012 01:48 PM

### CROSS REFERENCES

WO2003/088808, US2003/0228319; WO2003/062401 (claim 9); US2002/150573 (claim 4, pages 13-14); WO99/58658 (claim 13, FIG. 16); WO92/07574 (FIG. 1); U.S. Pat. No. 5,644,033; Ha et al (1992) J. Immunol. 148(5):1526-1531; Müller et al (1992) Eur. J. Immunol. 22:1621-1625; Hashimoto et al (1994) Immunogenetics 40(4):287-295; Preud'homme et al (1992) Clin. Exp. Immunol. 90(1):141-146; Yu et al (1992) J. Immunol. 148(2) 633-637; Sakaguchi et al (1988) EMBO J. 7(11):3457-3464 (29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pl: 8.54 MW: 41959 TM: 7 [P] Gene Chromosome: 11q23.3.

Nucleotide:
Genbank accession no NM_001716
Genbank version no. NM_001716.4 GI:342307092
Genbank record update date: Sep. 30, 2012 01:49 PM

Polypeptide:
Genbank accession no. NP_001707
Genbank version no. NP_001707.1 GI:4502415
Genbank record update date: Sep. 30, 2012 01:49 PM

### CROSS REFERENCES

WO2004/040000; WO2004/015426; US2003/105292 (Example 2); U.S. Pat. No. 6,555,339 (Example 2); WO2002/61087 (FIG. 1); WO2001/57188 (claim 20, page 269); WO2001/72830 (pages 12-13); WO2000/22129 (Example 1, pages 152-153, Example 2, pages 254-256); WO99/28468 (claim 1, page 38); U.S. Pat. No. 5,440,021 (Example 2, col 49-52); WO94/28931 (pages 56-58); WO92/17497 (claim 7, FIG. 5); Dobner et al (1992) Eur. J. Immunol. 22:2795-2799; Barella et al (1995) Biochem. J. 309:773-779 (30) HLA-DOB (Beta subunit of MHC class II molecule (Ia antigen) that binds peptides and presents them to CD4+ T lymphocytes); 273 aa, pl: 6.56, MW: 30820.TM: 1 [P] Gene Chromosome: 6p21.3)

Nucleotide:
Genbank accession no NM_002120
Genbank version no. NM_002120.3 GI:118402587
Genbank record update date: Sep. 8, 2012 04:46 PM

Polypeptide:
Genbank accession no. NP_002111
Genbank version no. NP_002111.1 GI:4504403
Genbank record update date: Sep. 8, 2012 04:46 PM

### CROSS REFERENCES

Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99:16899-16903; Servenius et al (1987) J. Biol. Chem. 262:8759-8766; Beck et al (1996) J. Mol. Biol. 255:1-13; Naruse et al (2002) Tissue Antigens 59:512-519; WO99/58658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al (1989) Immunogenetics 30(1):66-68; Larhammar et al (1985) J. Biol. Chem. 260(26):14111-14119 (31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).

Nucleotide:
Genbank accession no NM_002561
Genbank version no. NM_002561.3 GI:325197202
Genbank record update date: Jun. 27, 2012 12:41 AM

Polypeptide:
Genbank accession no. NP_002552
Genbank version no. NP_002552.2 GI:28416933
Genbank record update date: Jun. 27, 2012 12:41 AM

### CROSS REFERENCES

Le et al (1997) FEBS Lett. 418(1-2):195-199; WO2004/047749; WO2003/072035 (claim 10); Touchman et al (2000) Genome Res. 10:165-173; WO2002/22660 (claim 20); WO2003/093444 (claim 1); WO2003/087768 (claim 1); WO2003/029277 (page 82)

### (32) CD72 (B-Cell Differentiation Antigen CD72, Lyb-2); 359 aa, pl: 8.66, MW: 40225, TM: 1 [P] Gene Chromosome: 9p13.3).

Nucleotide:
Genbank accession no NM_001782
Genbank version no. NM_001782.2 GI:194018444
Genbank record update date: Jun. 26, 2012 01:43 PM

Polypeptide:
Genbank accession no. NP_001773
Genbank version no. NP_001773.1 GI:4502683
Genbank record update date: Jun. 26, 2012 01:43 PM

### CROSS REFERENCES

WO2004042346 (claim 65); WO2003/026493 (pages 51-52, 57-58); WO2000/75655 (pages 105-106); Von Hoegen et al (1990) J. Immunol. 144(12):4870-4877; Strausberg et al (2002) Proc. Natl. Acad. Sci USA 99:16899-16903.

(33) LY64 (Lymphocyte Antigen 64 (RP105), Type I Membrane Protein of the Leucine Rich Repeat (LRR) Family, Regulates B-Cell Activation and Apoptosis, Loss of Function is Associated with Increased Disease Activity in Patients with Systemic Lupus Erythematosis); 661 aa, pl: 6.20, MW: 74147 TM: 1 [P] Gene Chromosome: 5q12).

Nucleotide:
Genbank accession no NM_005582
Genbank version no. NM_005582.2 GI:167555126
Genbank record update date: Sep. 2, 2012 01:50 PM

Polypeptide:
Genbank accession no. NP_005573
Genbank version no. NP_005573.2 GI:167555127
Genbank record update date: Sep. 2, 2012 01:50 PM

### CROSS REFERENCES

US2002/193567; WO97/07198 (claim 11, pages 39-42); Miura et al (1996) Genomics 38(3):299-304; Miura et al (1998) Blood 92:2815-2822; WO2003/083047; WO97/44452 (claim 8, pages 57-61); WO2000/12130 (pages 24-26).

(34) FcRH1 (Fc Receptor-Like Protein 1, a Putative Receptor for the Immunoglobulin Fc Domain that Contains C2 Type Ig-Like and ITAM Domains, May have a Role in B-Lymphocyte Differentiation); 429 aa, pl: 5.28, MW: 46925 TM: 1 [P] Gene Chromosome: 1q21-1q22)

Nucleotide:
Genbank accession no NM_052938
Genbank version no. NM_052938.4 GI:226958543
Genbank record update date: Sep. 2, 2012 01:43 PM

Polypeptide:
Genbank accession no. NP_443170
Genbank version no. NP_443170.1 GI:16418419
Genbank record update date: Sep. 2, 2012 01:43 PM

### CROSS REFERENCES

WO2003/077836; WO2001/38490 (claim 6, FIG. 18E-1-18-E-2); Davis et al (2001) Proc. Natl. Acad. Sci USA 98(17):9772-9777; WO2003/089624 (claim 8); EP1347046 (claim 1); WO2003/089624 (claim 7).

(35) IRTA2 (Immunoglobulin Superfamily Receptor Translocation Associated 2, a Putative Immunoreceptor with Possible Roles in B Cell Development and Lymphomagenesis; Deregulation of the Gene by Translocation Occurs in Some B Cell Malignancies); 977 aa, pl: 6.88, MW: 106468, TM: 1 [P] Gene Chromosome: 1q21)

Nucleotide:
Genbank accession no AF343662
Genbank version no. AF343662.1 GI:13591709
Genbank record update date: Mar. 11, 2010 01:16 AM

Polypeptide:
Genbank accession no. AAK31325
Genbank version no. AAK31325.1 GI:13591710
Genbank record update date: Mar. 11, 2010 01:16 AM

### CROSS REFERENCES

AF343663, AF343664, AF343665, AF369794, AF397453, AK090423, AK090475, AL834187, AY358085; Mouse:AK089756, AY158090, AY506558; NP_112571.1; WO2003/024392 (claim 2, FIG. 97); Nakayama et al (2000) Biochem. Biophys. Res. Commun. 277(1):124-127; WO2003/077836; WO2001/38490 (claim 3, FIG. 18B-1-18B-2).

### (36) TENB2 (TMEFF2, Tomoregulin, TPEF, HPP1, TR, Putative Transmembrane Proteoglycan, Related to the EGF/Heregulin Family of Growth Factors and Follistatin); 374 aa)

Nucleotide:
Genbank accession no AF179274
Genbank version no. AF179274.2 GI:12280939
Genbank record update date: Mar. 11, 2010 01:05 AM

Polypeptide:
Genbank accession no. AAD55776
Genbank version no. AAD55776.2 GI:12280940
Genbank record update date: Mar. 11, 2010 01:05 AM

### CROSS REFERENCES

NCBI Accession: AAD55776, AAF91397, AAG49451, NCBI RefSeq: NP_057276; NCBI Gene: 23671; OMIM: 605734; SwissProt Q9UIK5; AY358907, CAF85723, CQ782436; WO2004/074320; JP2004113151; WO2003/042661; WO2003/009814; EP1295944 (pages 69-70); WO2002/30268 (page 329); WO2001/90304; US2004/249130; US2004/022727; WO2004/063355; US2004/197325; US2003/232350; US2004/005563; US2003/124579; Horie et al (2000) Genomics 67:146-152; Uchida et al (1999) Biochem. Biophys. Res. Commun. 266:593-602; Liang et al (2000) Cancer Res. 60:4907-12; Glynne-Jones et al (2001) Int J Cancer. October 15; 94(2):178-84.

### (37) PSMA—FOLH1 (Folate Hydrolase (Prostate-Specific Membrane Antigen) 1)

Nucleotide:
Genbank accession no M99487
Genbank version no. M99487.1 GI:190663
Genbank record update date: Jun. 23, 2010 08:48 AM

Polypeptide:
Genbank accession no. AAA60209
Genbank version no. AAA60209.1 GI:190664
Genbank record update date: Jun. 23, 2010 08:48 AM

### CROSS REFERENCES

Israeli R. S., et al Cancer Res. 53 (2), 227-230 (1993)

Other Information:
Official Symbol: FOLH1
Other Aliases: GIG27, FGCP, FOLH, GCP2, GCPII, NAALAD1, NAALAdase, PSM, PSMA, mGCP
Other Designations: N-acetylated alpha-linked acidic dipeptidase 1; N-acetylated-alpha-linked acidic dipeptidase I; NAALADase I; cell growth-inhibiting gene 27 protein; folylpoly-gamma-glutamate carboxypeptidase; glutamate carboxylase II; glutamate carboxypeptidase 2; glutamate carboxypeptidase II; membrane glutamate carboxypeptidase; prostate specific membrane antigen variant F; pteroylpoly-gamma-glutamate carboxypeptidase

Antibodies:
U.S. Pat. No. 7,666,425: Antibodies produces by Hybridomas having the following ATCC references:ATCC accession No. HB-12101, ATCC accession No. HB-12109, ATCC accession No. HB-12127 and ATCC accession No. HB-12126.
Proscan: a monoclonal antibody selected from the group consisting of 8H12, 3E11, 17G1, 2964, 30C1 and 20F2 (U.S. Pat. No. 7,811,564; Moffett S., et al Hybridoma (Larchmt). 2007 December; 26(6):363-72).
Cytogen: monoclonal antibodies 7E11-05 (ATCC accession No. HB 10494) and 9H10-A4 (ATCC accession No. HB11430)—U.S. Pat. No. 5,763,202
GlycoMimetics: NUH2—ATCC accession No. HB 9762 (U.S. Pat. No. 7,135,301)
Human Genome Science: HPRAJ70—ATCC accession No. 97131 (U.S. Pat. No. 6,824,993); Amino acid sequence encoded by the cDNA clone (HPRAJ70) deposited as American Type Culture Collection (“ATCC”) Deposit No. 97131
Medarex: Anti-PSMA antibodies that lack fucosyl residues—U.S. Pat. No. 7,875,278
Mouse anti-PSMA antibodies include the 3F5.4G6, 3D7.1.1, 4E10-1.14, 3E11, 4D8, 3E6, 3C9, 2C7, 1G3, 3C4, 3C6, 4D4, 1G9, 5C8B9, 3G6, 4C8B9, and monoclonal antibodies. Hybridomas secreting 3F5.4G6, 3D7.1.1, 4E10-1.14, 3E11, 4D8, 3E6, 3C9, 2C7, 1G3, 3C4, 3C6, 4D4, 1G9, 5C8B9, 3G6 or 4C8B9 have been publicly deposited and are described in U.S. Pat. No. 6,159,508. Relevant hybridomas have been publicly deposited and are described in U.S. Pat. No. 6,107,090. Moreover, humanized anti-PSMA antibodies, including a humanized version of J591, are described in further detail in PCT Publication WO 02/098897.
Other mouse anti-human PSMA antibodies have been described in the art, such as mAb 107-1A4 (Wang, S. et al. (2001) Int. J. Cancer 92:871-876) and mAb 2C9 (Kato, K. et al. (2003) Int. J. Urol. 10:439-444).
Examples of human anti-PSMA monoclonal antibodies include the 4A3, 7F12, 8C12, 8A11, 16F9, 2A10, 2C6, 2F5 and 1C3 antibodies, isolated and structurally characterized as originally described in PCT Publications WO 01/09192 and WO 03/064606 and in U.S. Provisional Application Ser. No. 60/654,125, entitled “Human Monoclonal Antibodies to Prostate Specific Membrane Antigen (PSMA)”, filed on Feb. 18, 2005. The V.sub.H amino acid sequences of 4A3, 7F12, 8C12, 8A11, 16F9, 2A10, 2C6, 2F5 and 1C3 are shown in SEQ ID NOs: 1-9, respectively. The V.sub.L amino acid sequences of 4A3, 7F12, 8C12, 8A11, 16F9, 2A10, 2C6, 2F5 and 1C3 are shown in SEQ ID NOs: 10-18, respectively.
Other human anti-PSMA antibodies include the antibodies disclosed in PCT Publication WO 03/034903 and US Application No. 2004/0033229.
NW Biotherapeutics: A hybridoma cell line selected from the group consisting of 3F5.4G6 having ATCC accession number HB12060, 3D7-1.I. having ATCC accession number HB12309, 4E10-1.14 having ATCC accession number HB12310, 3E11 (ATCC HB12488), 4D8 (ATCC HB12487), 3E6 (ATCC HB12486), 3C9 (ATCC HB12484), 2C7 (ATCC HB12490), 1G3 (ATCC HB12489), 3C4 (ATCC HB12494), 3C6 (ATCC HB12491), 4D4 (ATCC HB12493), 1G9 (ATCC HB12495), 5C8B9 (ATCC HB12492) and 3G6 (ATCC HB12485)—see U.S. Pat. No. 6,150,508
PSMA Development Company/Progenics/Cytogen—Seattle Genetics: mAb 3.9, produced by the hybridoma deposited under ATCC Accession No. PTA-3258 or mAb 10.3, produced by the hybridoma deposited under ATCC Accession No. PTA-3347—U.S. Pat. No. 7,850,971
PSMA Development Company—Compositions of PSMA antibodies (US 20080286284, Table 1)
This application is a divisional of U.S. patent application Ser. No. 10/395,894, filed on Mar. 21, 2003 (U.S. Pat. No. 7,850,971)
University Hospital Freiburg, Germany—mAbs 3/A12, 3/E7, and 3/F11 (Wolf P., et al Prostate. 2010 Apr. 1; 70(5):562-9).
   (38) SST (Somatostatin Receptor; Note that there Are5 Subtypes)

(38.1) SSTR2 (Somatostatin Receptor 2)

Nucleotide:
Genbank accession no NM_001050
Genbank version no. NM_001050.2 GI:44890054
Genbank record update date: Aug. 19, 2012 01:37 PM

Polypeptide:
Genbank accession no. NP_001041
Genbank version no. NP_001041.1 GI:4557859
Genbank record update date: Aug. 19, 2012 01:37 PM

### CROSS REFERENCES

Yamada Y., et al Proc. Natl. Acad. Sci. U.S.A. 89 (1), 251-255 (1992); Susini C., et al Ann Oncol. 2006 December; 17(12):1733-42

Other Information:
Official Symbol: SSTR2
Other Designations: SRIF-1; SS2R; somatostatin receptor type 2

(38.2) SSTR5 (Somatostatin Receptor 5)

Nucleotide:
Genbank accession no D16827
Genbank version no. D16827.1 GI:487683
Genbank record update date: Aug. 1, 2006 12:45 PM

Polypeptide:
Genbank accession no. BAA04107
Genbank version no. BAA04107.1 GI:487684
Genbank record update date: Aug. 1, 2006 12:45 PM

### CROSS REFERENCES

Yamada, Y., et al Biochem. Biophys. Res. Commun. 195 (2), 844-852 (1993)

Other Information:
Official Symbol: SSTR5
Other Aliases: SS-5-R
Other Designations: Somatostatin receptor subtype 5; somatostatin receptor type 5

(38.3) SSTR1

(38.4) SSTR3

(38.5) SSTR4
AvB6—Both subunits (39+40)

### (39) ITGAV (Integrin, Alpha V)

Nucleotide:
Genbank accession no M14648 J02826 M18365
Genbank version no. M14648.1 GI:340306
Genbank record update date: Jun. 23, 2010 08:56 AM

Polypeptide:
Genbank accession no. AAA36808
Genbank version no. AAA36808.1 GI:340307
Genbank record update date: Jun. 23, 2010 08:56 AM

### CROSS REFERENCES

Suzuki S., et al Proc. Natl. Acad. Sci. U.S.A. 83 (22), 8614-8618 (1986)

Other Information:
Official Symbol: ITGAV
Other Aliases: CD51, MSK8, VNRA, VTNR
Other Designations: antigen identified by monoclonal antibody L230; integrin alpha-V; integrin alphaVbeta3; integrin, alpha V (vitronectin receptor, alpha polypeptide, antigen CD51); vitronectin receptor subunit alpha

### (40) ITGB6 (Integrin, Beta 6)

Nucleotide:
Genbank accession no NM_000888
Genbank version no. NM_000888.3 GI:9966771
Genbank record update date: Jun. 27, 2012 12:46 AM

Polypeptide:
Genbank accession no. NP_000879
Genbank version no. NP_000879.2 GI:9625002
Genbank record update date: Jun. 27, 2012 12:46 AM

### CROSS REFERENCES

Sheppard D. J., et al Biol. Chem. 265 (20), 11502-11507 (1990)

Other Information:
Official Symbol: ITGB6
Other Designations: integrin beta-6

Antibodies:
Biogen: U.S. Pat. No. 7,943,742—Hybridoma clones 6.3G9 and 6.8G6 were deposited with the ATCC, accession numbers ATCC PTA-3649 and -3645, respectively.
Biogen: U.S. Pat. No. 7,465,449—In some embodiments, the antibody comprises the same heavy and light chain polypeptide sequences as an antibody produced by hybridoma 6.1A8, 6.3G9, 6.8G6, 6.261, 6.2610, 6.2A1, 6.2E5, 7.1G10, 7.7G5, or 7.105.
Centocor (J&J): U.S. Pat. Nos. 7,550,142; 7,163,681
   for example in U.S. Pat. No. 7,550,142—an antibody having human heavy chain and human light chain variable regions comprising the amino acid sequences shown in SEQ ID NO: 7 and SEQ ID NO: 8.
Seattle Genetics: 15H3 (Ryan M C., et al Cancer Res Apr. 15, 2012; 72(8 Supplement): 4630)

### (41) CEACAM5 (Carcinoembryonic Antigen-Related Cell Adhesion Molecule 5)

Nucleotide:
Genbank accession no M17303
Genbank version no. M17303.1 GI:178676
Genbank record update date: Jun. 23, 2010 08:47 AM

Polypeptide:
Genbank accession no. AAB59513
Genbank version no. AAB59513.1 GI:178677
Genbank record update date: Jun. 23, 2010 08:47 AM

### CROSS REFERENCES

Beauchemin N., et al Mol. Cell. Biol. 7 (9), 3221-3230 (1987)

Other Information:
Official Symbol: CEACAM5
Other Aliases: CD66e, CEA
Other Designations: meconium antigen 100

Antibodies:
AstraZeneca-MedImmune: US 20100330103; US20080057063; US20020142359
   for example an antibody having complementarity determining regions (CDRs) with the following sequences: heavy chain; CDR1—DNYMH, CDR2—WIDPENGDTE YAPKFRG, CDR3—LIYAGYLAMD Y; and light chain CDR1—SASSSVTYMH, CDR2—STSNLAS, CDR3—QQRSTYPLT.
   Hybridoma 806.077 deposited as European Collection of Cell Cultures (ECACC) deposit no. 96022936.
Research Corporation Technologies, Inc.: U.S. Pat. No. 5,047,507
Bayer Corporation: U.S. Pat. No. 6,013,772
BioAlliance: U.S. Pat. Nos. 7,982,017; 7,674,605
U.S. Pat. No. 7,674,605
   an antibody comprising the heavy chain variable region sequence from the amino acid sequence of SEQ ID NO: 1, and the light chain variable region sequence from the amino acid sequence of SEQ ID NO:2.
   an antibody comprising the heavy chain variable region sequence from the amino acid sequence of SEQ ID NO:5, and the light chain variable region sequence from the amino acid sequence of SEQ ID NO:6.
Celltech Therapeutics Limited: U.S. Pat. No. 5,877,293
The Dow Chemical Company: U.S. Pat. Nos. 5,472,693; 6,417,337; 6,333,405
U.S. Pat. No. 5,472,693—for example, ATCC No. CRL-11215
U.S. Pat. No. 6,417,337—for example, ATCC CRL-12208
U.S. Pat. No. 6,333,405—for example, ATCC CRL-12208
Immunomedics, Inc: U.S. Pat. Nos. 7,534,431; 7,230,084; 7,300,644; 6,730,300; US20110189085
   an antibody having CDRs of the light chain variable region comprise: CDR1 comprises KASQDVGTSVA (SEQ ID NO: 20); CDR2 comprises WTSTRHT (SEQ ID NO: 21); and CDR3 comprises QQYSLYRS (SEQ ID NO: 22); and the CDRs of the heavy chain variable region of said anti-CEA antibody comprise: CDR1 comprises TYWMS (SEQ ID NO: 23); CDR2 comprises EIHPDSSTINYAPSLKD (SEQ ID NO: 24); and CDR3 comprises LYFGFPWFAY (SEQ ID NO: 25).
US20100221175; US20090092598; US20070202044; US20110064653; US20090185974; US20080069775.

### (42) MET (Met Proto-Oncogene; Hepatocyte Growth Factor Receptor)

Nucleotide:
Genbank accession no M35073
Genbank version no. M35073.1 GI:187553
Genbank record update date: Mar. 6, 2012 11:12 AM

Polypeptide:
Genbank accession no. AAA59589
Genbank version no. AAA59589.1 GI:553531
Genbank record update date: Mar. 6, 2012 11:12 AM

### CROSS REFERENCES

Dean M., et al Nature 318 (6044), 385-388 (1985)

Other Information:
Official Symbol: MET
Other Aliases: AUTS9, HGFR, RCCP2, c-Met
Other Designations: HGF receptor; HGF/SF receptor; SF receptor; hepatocyte growth factor receptor; met proto-oncogene tyrosine kinase; proto-oncogene c-Met; scatter factor receptor; tyrosine-protein kinase Met

Antibodies:
Abgenix/Pfizer: US20100040629
   for example, the antibody produced by hybridoma 13.3.2 having American Type Culture Collection (ATCC) accession number PTA-5026; the antibody produced by hybridoma 9.1.2 having ATCC accession number PTA-5027; the antibody produced by hybridoma 8.70.2 having ATCC accession number PTA-5028; or the antibody produced by hybridoma 6.90.3 having ATCC accession number PTA-5029.
Amgen/Pfizer: US20050054019
   for example, an antibody comprising a heavy chain having the amino acid sequences set forth in SEQ ID NO: 2 where X2 is glutamate and X4 is serine and a light chain having the amino acid sequence set forth in SEQ ID NO: 4 where X8 is alanine, without the signal sequences; an antibody comprising a heavy chain having the amino acid sequences set forth in SEQ ID NO: 6 and a light chain having the amino acid sequence set forth in SEQ ID NO: 8, without the signal sequences; an antibody comprising a heavy chain having the amino acid sequences set forth in SEQ ID NO: 10 and a light chain having the amino acid sequence set forth in SEQ ID NO: 12, without the signal sequences; or an antibody comprising a heavy chain having the amino acid sequences set forth in SEQ ID NO: 14 and a light chain having the amino acid sequence set forth in SEQ ID NO: 16, without the signal sequences.
Agouron Pharmaceuticals (Now Pfizer): US20060035907
Eli Lilly: US20100129369
Genentech: U.S. Pat. No. 5,686,292; US20100028337; US20100016241; US20070129301; US20070098707; US20070092520, US20060270594; US20060134104; US20060035278; US20050233960; US20050037431 U.S. Pat. No. 5,686,292—for example, ATCC HB-11894 and ATCC HB-11895 US 20100016241—for example, ATCC HB-11894 (hybridoma 1A3.3.13) or HB-11895 (hybridoma 5D5.11.6)
National Defense Medical Center, Taiwan: Lu R M., et al Biomaterials. 2011 April; 32(12):3265-74.
Novartis: US20090175860
   for example, an antibody comprising the sequences of CDR1, CDR2 and CDR3 of heavy chain 4687, wherein the sequences of CDR1, CDR2, and CDR3 of heavy chain 4687 are residues 26-35, 50-65, and 98-102, respectively, of SEQ ID NO: 58; and the sequences of CDR1, CDR2, and CDR3 of light chain 5097, wherein the sequences of CDR1, CDR2, and CDR3 of light chain 5097 are residues 24-39, 55-61, and 94-100 of SEQ ID NO: 37.
Pharmacia Corporation: US20040166544
Pierre Fabre: US20110239316, US20110097262, U520100115639
Sumsung: US 20110129481—for example a monoclonal antibody produced from a hybridoma cell having accession number KCLRF-BP-00219 or accession number of KCLRF-BP-00223.
Samsung: US 20110104176—for example an antibody produced by a hybridoma cell having Accession Number: KCLRF-BP-00220.
University of Turin Medical School: DN-30 Pacchiana G., et al J Biol Chem. 2010 Nov. 12; 285(46):36149-57
Van Andel Research Institute: Jiao Y., et al Mol Biotechnol. 2005 September; 31(1):41-54.

### (43) MUC1 (Mucin 1, Cell Surface Associated)

Nucleotide:
Genbank accession no J05581
Genbank version no. J05581.1 GI:188869
Genbank record update date: Jun. 23, 2010 08:48 AM

Polypeptide:
Genbank accession no. AAA59876
Genbank version no. AAA59876.1 GI:188870
Genbank record update date: Jun. 23, 2010 08:48 AM

### CROSS REFERENCES

Gendler S. J., et al J. Biol. Chem. 265 (25), 15286-15293 (1990)

Other Information:
Official Symbol: MUC1
Other Aliases: RP11-263K19.2, CD227, EMA, H23AG, KL-6, MAM6, MUC-1, MUC-1/SEC, MUC-1/X, MUC1/ZD, PEM, PEMT, PUM
Other Designations: DF3 antigen; H23 antigen; breast carcinoma-associated antigen DF3; carcinoma-associated mucin; episialin; krebs von den Lungen-6; mucin 1, transmembrane; mucin-1; peanut-reactive urinary mucin; polymorphic epithelial mucin; tumor associated epithelial mucin; tumor-associated epithelial membrane antigen; tumor-associated mucin

Antibodies:
AltaRex—Quest Pharma Tech: U.S. Pat. No. 6,716,966—for example an Alt-1 antibody produced by the hybridoma ATCC No PTA-975.
AltaRex—Quest Pharma Tech: U.S. Pat. No. 7,147,850
CRT: 5E5—Sorensen A L., et al Glycobiology vol. 16 no. 2 pp. 96-107, 2006; HMFG2—Burchell J., et al Cancer Res., 47, 5476-5482 (1987); see WO2015/159076
Glycotope GT-MAB: GT-MAB 2.5-GEX (Website: http://www.glycotope.com/pipeline/pankomab-gex)
Immunogen: U.S. Pat. No. 7,202,346
   for example, antibody MJ-170: hybridoma cell line MJ-170 ATCC accession no. PTA-5286Monoclonal antibody MJ-171: hybridoma cell line MJ-171 ATCC accession no. PTA-5287; monoclonal antibody MJ-172: hybridoma cell line MJ-172 ATCC accession no. PTA-5288; or monoclonal antibody MJ-173: hybridoma cell line MJ-173 ATCC accession no. PTA-5302
Immunomedics: U.S. Pat. No. 6,653,104
Ramot Tel Aviv Uni: U.S. Pat. No. 7,897,351
Regents Uni. CA: U.S. Pat. No. 7,183,388; US20040005647; US20030077676.
Roche GlycArt: U.S. Pat. No. 8,021,856
Russian National Cancer Research Center: Imuteran-Ivanov P K., et al Biotechnol J. 2007 July; 2(7):863-70
Technische Univ Braunschweig: (1166, HT186-B7, HT186-D11, HT186-G2, HT200-3A-C1, HT220-M-D1, HT220-M-G8)—Thie H., et al PLoS One. 2011 Jan. 14; 6(1):e15921

### (44) CA9 (Carbonic Anhydrase IX)

Nucleotide:
Genbank accession no. X66839
Genbank version no. X66839.1 GI:1000701
Genbank record update date: Feb. 2, 2011 10:15 AM

Polypeptide:
Genbank accession no. CAA47315
Genbank version no. CAA47315.1 GI:1000702
Genbank record update date: Feb. 2, 2011 10:15 AM

### CROSS REFERENCES

Pastorek J., et al Oncogene 9 (10), 2877-2888 (1994)

Other Information:
Official Symbol: CA9
Other Aliases: CAIX, MN
Other Designations: CA-IX; P54/58N; RCC-associated antigen G250; RCC-associated protein G250; carbonate dehydratase IX; carbonic anhydrase 9; carbonic dehydratase; membrane antigen MN; pMW1; renal cell carcinoma-associated antigen G250

Antibodies:
Abgenix/Amgen: US20040018198
Affibody: Anti-CAIX Affibody molecules (http://www.affibody.com/en/Product-Portfolio/Pipeline/)
Bayer: U.S. Pat. No. 7,462,696
Bayer/Morphosys: 3ee9 mAb—Petrul H M., et al Mol Cancer Ther. 2012 February; 11(2):340-9
Harvard Medical School: Antibodies G10, G36, G37, G39, G45, G57, G106, G119, G6, G27, G40 and G125. Xu C., et al PLoS One. 2010 Mar. 10; 5(3):e9625
Institute of Virology, Slovak Academy of Sciences (Bayer)—U.S. Pat. No. 5,955,075
   for example, M75—ATCC Accession No. HB 11128 or MN12—ATCC Accession No. HB 11647
Institute of Virology, Slovak Academy of Sciences: U.S. Pat. No. 7,816,493
   for example the M75 monoclonal antibody that is secreted from the hybridoma VU-M75, which was deposited at the American Type Culture Collection under ATCC No. HB 11128; or the V/10 monoclonal antibody secreted from the hybridoma V/10-VU, which was deposited at the International Depository Authority of the Belgian Coordinated Collection of Microorganisms (BCCM) at the Laboratorium voor Moleculaire Biologie-Plasmidencollectie (LMBP) at the Universeit Gent in Gent, Belgium, under Accession No. LMBP 6009CB.
Institute of Virology, Slovak Academy of Sciences US20080177046; U520080176310; U520080176258; U520050031623
Novartis: US20090252738
Wilex: U.S. Pat. No. 7,691,375—for example the antibody produced by the hybridoma cell line DSM ASC 2526.
Wilex: US20110123537; Rencarex: Kennett R H., et al Curr Opin Mol Ther. 2003 February; 5(1):70-5
Xencor: US20090162382

### (45) EGFRvIII (Epidermal Growth Factor Receptor (EGFR), Transcript Variant 3

Nucleotide:
Genbank accession no. NM_201283
Genbank version no. NM_201283.1 GI:41327733
Genbank record update date: Sep. 30, 2012 01:47 PM

Polypeptide:
Genbank accession no. NP_958440
Genbank version no. NP_958440.1 GI:41327734
Genbank record update date: Sep. 30, 2012 01:47 PM

### CROSS-REFERENCES

Batra S K., et al Cell Growth Differ 1995; 6:1251-1259.

Antibodies:
U.S. Pat. Nos. 7,628,986 and 7,736,644 (Amgen)
   for example, a heavy chain variable region amino acid sequence selected from the group consisting of SEQ ID NO: 142 and variants & a light chain variable region amino acid sequence selected from the group consisting of: SEQ ID NO: 144 and variants.
US20100111979 (Amgen)
   for example, an antibody comprising a heavy chain amino acid sequence comprising: CDR1 consisting of a sequence selected from the group consisting of the amino acid sequences for the CDR1 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17); CDR2 consisting of a sequence selected from the group consisting of the amino acid sequences for the CDR2 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17); and CDR3 consisting of a sequence selected from the group consisting of the amino acid sequences for the CDR3 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).
US20090240038 (Amgen)
   for example, an antibody having at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.
US20090175887 (Amgen)
   for example, an antibody having a heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).
US20090156790 (Amgen)
   for example, antibody having heavy chain polypeptide and a light chain polypeptide, wherein at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.
US20090155282, US20050059087 and US20050053608 (Amgen)
   for example, an antibody heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).
MR1-1 (U.S. Pat. No. 7,129,332; Duke)
   for example, a variant antibody having the sequence of SEQ ID NO.18 with the substitutions S98P-T99Y in the CDR3 VH, and F92W in CDR3 VL.
L8A4, H10, Y10 (Wikstrand C J., et al Cancer Res. 1995 Jul. 15; 55(14):3140-8; Duke)
US20090311803 (Harvard University)
   for example, SEQ ID NO:9 for antibody heavy chain variable region, and SEQ ID NO: 3 for light chain variable region amino acid sequences
US20070274991 (EMD72000, also known as matuzumab; Harvard University)
   for example, SEQ ID NOs: 3 & 9 for light chain and heavy chain respectively
U.S. Pat. No. 6,129,915 (Schering)
   for example, SEQ. ID NOs: 1, 2, 3, 4, 5 and 6.
mAb CH12—Wang H., et al FASEB J. 2012 January; 26(1):73-80 (Shanghai Cancer Institute).
RAbDMvIII—Gupta P., et al BMC Biotechnol. 2010 Oct. 7; 10:72 (Stanford University Medical Center).
mAb Ua30—Ohman L., et al Tumour Biol. 2002 March-April; 23(2):61-9 (Uppsala University).
Han D G., et al Nan Fang Yi Ke Da Xue Xue Bao. 2010 January; 30(1):25-9 (Xi'an Jiaotong University).

### (46) CD33 (CD33 Molecule)

Nucleotide:
Genbank accession no. M_23197
Genbank version no. NM_23197.1 GI:180097
Genbank record update date: Jun. 23, 2010 08:47 AM

Polypeptide:
Genbank accession no. AAA51948
Genbank version no. AAA51948.1 GI:188098
Genbank record update date: Jun. 23, 2010 08:47 AM

### CROSS-REFERENCES

Simmons D., et al J. Immunol. 141 (8), 2797-2800 (1988)

Other Information:
Official Symbol: CD33
Other Aliases: SIGLEC-3, SIGLEC3, p67
Other Designations: CD33 antigen (gp67); gp67; myeloid cell surface antigen CD33; sialic acid binding Ig-like lectin 3; sialic acid-binding Ig-like lectin

Antibodies:
H195 (Lintuzumab)—Raza A., et al Leuk Lymphoma. 2009 August; 50(8):1336-44; U.S. Pat. No. 6,759,045 (Seattle Genetics/Immunomedics)
mAb OKT9: Sutherland, D. R. et al. Proc Natl Acad Sci USA 78(7): 4515-4519 1981, Schneider, C., et al J Biol Chem 257, 8516-8522 (1982) mAb E6: Hoogenboom, H. R., et al J Immunol 144, 3211-3217 (1990)
U.S. Pat. No. 6,590,088 (Human Genome Sciences)
   for example, SEQ ID NOs: 1 and 2 and ATCC accession no. 97521
U.S. Pat. No. 7,557,189 (Immunogen)
   for example, an antibody or fragment thereof comprising a heavy chain variable region which comprises three CDRs having the amino acid sequences of SEQ ID NOs:1-3 and a light chain variable region comprising three CDRs having the amino acid sequences of SEQ ID NOs:4-6.

### (47) CD19 (CD19 Molecule)

Nucleotide:
Genbank accession no. NM_001178098
Genbank version no. NM_001178098.1 GI:296010920
Genbank record update date: Sep. 10, 2012 12:43 AM

Polypeptide:
Genbank accession no. NP_001171569
Genbank version no. NP_001171569.1 GI:296010921
Genbank record update date: Sep. 10, 2012 12:43 AM

### CROSS-REFERENCES

Tedder T F., et al J. Immunol. 143 (2): 712-7 (1989)

Other Information:
Official Symbol: CD19
Other Aliases: B4, CVID3
Other Designations: B-lymphocyte antigen CD19; B-lymphocyte surface antigen B4; T-cell surface antigen Leu-12; differentiation antigen CD19

Antibodies:
Immunogen: HuB4—Al-Katib A M., et al Clin Cancer Res. 2009 Jun. 15; 15(12):4038-45.
4G7: Kügler M., et al Protein Eng Des Sel. 2009 March; 22(3):135-47
   for example, sequences in FIG. 3 of of Knappik, A. et al. J Mol Biol 2000 February; 296(1):57-86
AstraZeneca/MedImmune: MEDI-551—Herbst R., et al J Pharmacol Exp Ther. 2010 October; 335(1):213-22
Glenmark Pharmaceuticals: GBR-401—Hou S., et al Mol Cancer Ther November 2011 (Meeting Abstract Supplement) C164
U.S. Pat. No. 7,109,304 (Immunomedics)
   for example, an antibody comprising the sequence of hA19Vk (SEQ ID NO:7) and the sequence of hA19VH (SEQ ID NO:10)
U.S. Pat. No. 7,902,338 (Immunomedics)
   for example, an antibody or antigen-binding fragment thereof that comprises the light chain complementarity determining region CDR sequences CDR1 of SEQ ID NO: 16 (KASQSVDYDGDSYLN); CDR2 of SEQ ID NO: 17 (DASNLVS); and CDR3 of SEQ ID NO: 18 (QQSTEDPWT) and the heavy chain CDR sequences CDR1 of SEQ ID NO: 19 (SYWMN); CDR2 of SEQ ID NO: 20 (QIWPGDGDTNYNGKFKG) and CDR3 of SEQ ID NO: 21 (RETTTVGRYYYAMDY) and also comprises human antibody framework (FR) and constant region sequences with one or more framework region amino acid residues substituted from the corresponding framework region sequences of the parent murine antibody, and wherein said substituted FR residues comprise the substitution of serine for phenylalanine at Kabat residue 91 of the heavy chain variable region.
Medarex: MDX-1342—Cardarelli P M., et al Cancer Immunol Immunother. 2010 February; 59(2):257-65.
MorphoSys/Xencor: MOR-208/XmAb-5574—Zalevsky J., et al Blood. 2009 Apr. 16; 113(16):3735-43
U.S. Pat. No. 7,968,687 (Seattle Genetics)
   An antibody or antigen-binding fragment comprising a heavy chain variable domain comprising the amino acid sequence of SEQ ID NO:9 and a light chain variable domain comprising the amino acid sequence of SEQ ID NO: 24.
4G7 chim—Lang P., et al Blood. 2004 May 15; 103(10):3982-5 (University of Tübingen)
   for example, FIG. 6 and SEQ ID No: 80 of US20120082664
Zhejiang University School of Medicine: 2E8—Zhang J., et al J Drug Target. 2010 November; 18(9):675-8

### (48) IL2RA (Interleukin 2 Receptor, Alpha); NCBI Reference Sequence: NM_000417.2)

Nucleotide:
Genbank accession no. NM_000417
Genbank version no. NM_000417.2 GI:269973860
Genbank record update date: Sep. 9, 2012 04:59 PM

Polypeptide:
Genbank accession no. NP_000408
Genbank version no. NP_000408.1 GI:4557667
Genbank record update date: Sep. 9, 2012 04:59 PM

### CROSS-REFERENCES

Kuziel W. A., et al J. Invest. Dermatol. 94 (6 SUPPL), 27S-32S (1990)

Other Information:
Official Symbol: IL2RA
Other Aliases: RP11-536K7.1, CD25, IDDM10, IL2R, TCGFR
Other Designations: FIL-2 receptor subunit alpha; IL-2-RA; IL-2R subunit alpha; IL2-RA; TAC antigen; interleukin-2 receptor subunit alpha; p55

Antibodies:
U.S. Pat. No. 6,383,487 (Novartis/UCL: Baxilisimab [Simulect])
U.S. Pat. No. 6,521,230 (Novartis/UCL: Baxilisimab [Simulect])
   for example, an antibody having an antigen binding site comprises at least one domain which comprises CDR1 having the amino acid sequence in SEQ. ID. NO: 7, CDR2 having the amino acid sequence in SEQ. ID. NO: 8, and CDR3 chaving the amino acid sequence in SEQ. ID. NO: 9; or said CDR1, CDR2 and CDR3 taken in sequence as a whole comprise an amino acid sequence which is at least 90% identical to SEQ. ID. NOs: 7, 8 and 9 taken in sequence as a whole.
Daclizumab—Rech A J., et al Ann N Y Acad Sci. 2009 September; 1174:99-106 (Roche)

### (49) AXL (AXL Receptor Tyrosine Kinase)

Nucleotide:
Genbank accession no. M76125
Genbank version no. M76125.1 GI:292869
Genbank record update date: Jun. 23, 2010 08:53 AM

Polypeptide:
Genbank accession no. AAA61243
Genbank version no. AAA61243.1 GI:29870
Genbank record update date: Jun. 23, 2010 08:53 AM

### CROSS-REFERENCES

O'Bryan J. P., et al Mol. Cell. Biol. 11 (10), 5016-5031 (1991); Bergsagel P. L., et al J. Immunol. 148 (2), 590-596 (1992)

Other Information:
Official Symbol: AXL
Other Aliases: JTK11, UFO
Other Designations: AXL oncogene; AXL transforming sequence/gene; oncogene AXL; tyrosine-protein kinase receptor UFO

Antibodies:
YW327.652—Ye X., et al Oncogene. 2010 Sep. 23; 29(38):5254-64. (Genentech)
BergenBio: BGB324 (http://www.bergenbio.com/BGB324)

### (50) CD30—TNFRSF8 (Tumor Necrosis Factor Receptor Superfamily, Member 8)

Nucleotide:
Genbank accession no. M83554
Genbank version no. M83554.1 GI:180095
Genbank record update date: Jun. 23, 2010 08:53 AM

Polypeptide:
Genbank accession no. AAA51947
Genbank version no. AAA51947.1 GI:180096
Genbank record update date: Jun. 23, 2010 08:53 AM

### CROSS-REFERENCES

Durkop H., et al Cell 68 (3), 421-427 (1992)

Other Information:
Official Symbol: TNFRSF8
Other Aliases: CD30, D1S166E, Ki-1
Other Designations: CD30L receptor; Ki-1 antigen; cytokine receptor CD30; lymphocyte activation antigen CD30; tumor necrosis factor receptor superfamily member 8

### (51) BCMA (B-Cell Maturation Antigen)—TNFRSF17 (Tumor Necrosis Factor Receptor Superfamily, Member 17)

Nucleotide:
Genbank accession no. Z29574
Genbank version no. Z29574.1 GI:471244
Genbank record update date: Feb. 2, 2011 10:40 AM

Polypeptide:
Genbank accession no. CAA82690
Genbank version no. CAA82690.1 GI:471245
Genbank record update date: Feb. 2, 2011 10:40 AM

### CROSS-REFERENCES

Laabi Y., et al Nucleic Acids Res. 22 (7), 1147-1154 (1994)

Other Information:
Official Symbol: TNFRSF17
Other Aliases: BCM, BCMA, CD269
Other Designations: B cell maturation antigen; B-cell maturation factor; B-cell maturation protein; tumor necrosis factor receptor superfamily member 17

### (52) CT Ags—CTA (Cancer Testis Antigens)

### CROSS-REFERENCES

Fratta E., et al. Mol Oncol. 2011 April; 5(2):164-82; Lim S H., at al Am J Blood Res. 2012; 2(1):29-35.

### (53) CD174 (Lewis Y)—FUT3 (Fucosyltransferase 3 (Galactoside 3(4)-L-Fucosyltransferase, Lewis Blood Group)

Nucleotide:
Genbank accession no. NM000149
Genbank version no. NM000149.3 GI:148277008
Genbank record update date: Jun. 26, 2012 04:49 PM

Polypeptide:
Genbank accession no. NP_000140
Genbank version no. NP_000140.1 GI:4503809
Genbank record update date: Jun. 26, 2012 04:49 PM

### CROSS-REFERENCES

Kukowska-Latallo, J. F., et al Genes Dev. 4 (8), 1288-1303 (1990)

Other Information:
Official Symbol: FUT3
Other Aliases: CD174, FT3B, FucT-Ill, LE, Les
Other Designations: Lewis F T; alpha-(1,3/1,4)-fucosyltransferase; blood group Lewis alpha-4-fucosyltransferase; fucosyltransferase III; galactoside 3(4)-L-fucosyltransferase
   (54) CLEC14A (C-Type Lectin Domain Family 14, Member a; Genbank Accession No. NM175060)

Nucleotide:
Genbank accession no. NM175060
Genbank version no. NM175060.2 GI:371123930
Genbank record update date: Apr. 1, 2012 03:34 PM

Polypeptide:
Genbank accession no. NP_778230
Genbank version no. NP_778230.1 GI:28269707
Genbank record update date: Apr. 1, 2012 03:34 PM

Other Information:
Official Symbol: CLEC14A
Other Aliases: UNQ236/PRO269, C14orf27, CEG1, EGFR-5
Other Designations: C-type lectin domain family 14 member A; CIECT and EGF-like domain containing protein; epidermal growth factor receptor 5 (55) GRP78—HSPA5 (heat shock 70 kDa protein 5 (glucose-regulated protein, 78 kDa)

Nucleotide:
Genbank accession no. NM005347
Genbank version no. NM005347.4 GI:305855105
Genbank record update date: Sep. 30, 2012 01:42 PM

Polypeptide:
Genbank accession no. NP_005338
Genbank version no. NP_005338.1 GI:16507237
Genbank record update date: Sep. 30, 2012 01:42 PM

### CROSS-REFERENCES

Ting J., et al DNA 7 (4), 275-286 (1988)

Other Information:
Official Symbol: HSPA5
Other Aliases: BIP, GRP78, MIF2
Other Designations: 78 kDa glucose-regulated protein; endoplasmic reticulum lumenal Ca(2+)-binding protein grp78; immunoglobulin heavy chain-binding protein

### (56) CD70 (CD70 Molecule) L08096

Nucleotide:
Genbank accession no. L08096
Genbank version no. L08096.1 GI:307127
Genbank record update date: Jun. 23, 2012 08:54 AM

Polypeptide:
Genbank accession no. AAA36175
Genbank version no. AAA36175.1 GI:307128
Genbank record update date: Jun. 23, 2012 08:54 AM

### CROSS-REFERENCES

Goodwin R. G., et al Cell 73 (3), 447-456 (1993)

Other Information:
Official Symbol: CD70
Other Aliases: CD27L, CD27LG, TNFSF7
Other Designations: CD27 ligand; CD27-L; CD70 antigen; Ki-24 antigen; surface antigen CD70; tumor necrosis factor (ligand) superfamily, member 7; tumor necrosis factor ligand superfamily member 7

Antibodies:
MDX-1411 against CD70 (Medarex)
h1F6 (Oflazoglu, E., et al, Clin Cancer Res. 2008 Oct. 1; 14(19):6171-80; Seattle Genetics)
   for example, see US20060083736 SEQ ID NOs: 1, 2, 11 and 12 and FIG. 1.

### (57) Stem Cell Specific Antigens.

### For Example

5T4 (see entry (63) below)

CD25 (see entry (48) above)

CD32

Polypeptide:
Genbank accession no. ABK42161
Genbank version no. ABK42161.1 GI:117616286
Genbank record update date: Jul. 25, 2007 03:00 PM

LGR5/GPR49

Nucleotide:
Genbank accession no. NM_003667
Genbank version no. NM_003667.2 GI:24475886
Genbank record update date: Jul. 22, 2012 03:38 PM

Polypeptide:
Genbank accession no. NP_003658
Genbank version no. NP_003658.1 GI:4504379
Genbank record update date: Jul. 22, 2012 03:38 PM

Prominin/CD133

Nucleotide:
Genbank accession no. NM_006017
Genbank version no. NM_006017.2 GI:224994187
Genbank record update date: Sep. 30, 2012 01:47 PM

Polypeptide:
Genbank accession no. NP_006008
Genbank version no. NP_006008.1 GI:5174387
Genbank record update date: Sep. 30, 2012 01:47 PM

### (58) ASG-5

### CROSS-REFERENCES

(Smith L. M., et. al AACR 2010 Annual Meeting (abstract #2590); Gudas J. M., et. al. AACR 2010 Annual Meeting (abstract #4393)

Antibodies:
Anti-AGS-5 Antibody: M6.131 (Smith, L. M., et. al AACR 2010 Annual Meeting (abstract #2590)

### (59) ENPP3 (Ectonucleotide Pyrophosphatase/Phosphodiesterase 3)

Nucleotide:
Genbank accession no. AF005632
Genbank version no. AF005632.2 GI:4432589
Genbank record update date: Mar. 10, 2010 09:41 PM

Polypeptide:
Genbank accession no. AAC51813
Genbank version no. AAC51813.1 GI:2465540
Genbank record update date: Mar. 10, 2010 09:41 PM

### CROSS-REFERENCES

Jin-Hua P., et al Genomics 45 (2), 412-415 (1997)

Other Information:
Official Symbol: ENPP3
Other Aliases: RP5-988G15.3, B10, CD203c, NPP3, PD-IBETA, PDNP3
Other Designations: E-NPP 3; dJ1005H11.3 (phosphodiesterase I/nucleotide pyrophosphatase 3); dJ914N13.3 (phosphodiesterase I/nucleotide pyrophosphatase 3); ectonucleotide pyrophosphatase/phosphodiesterase family member 3; gp130RB13-6; phosphodiesterase I beta; phosphodiesterase I/nucleotide pyrophosphatase 3; phosphodiesterase-I beta

### (60) PRR4 (Proline Rich 4 (Lacrimal))

Nucleotide:
Genbank accession no. NM_007244
Genbank version no. NM_007244.2 GI:154448885
Genbank record update date: Jun. 28, 2012 12:39 PM

Polypeptide:
Genbank accession no. NP_009175
Genbank version no. NP_009175.2 GI:154448886
Genbank record update date: Jun. 28, 2012 12:39 PM

### CROSS-REFERENCES

Dickinson D. P., et al Invest. Ophthalmol. Vis. Sci. 36 (10), 2020-2031 (1995)

Other Information:
Official Symbol: PRR4
Other Aliases: LPRP, PROL4
Other Designations: lacrimal proline-rich protein; nasopharyngeal carcinoma-associated proline-rich protein 4; proline-rich polypeptide 4; proline-rich protein 4

### (61) GCC—GUCY2C (Guanylate Cyclase 2C (Heat Stable Enterotoxin Receptor)

Nucleotide:
Genbank accession no. NM_004963
Genbank version no. NM_004963.3 GI:222080082
Genbank record update date: Sep. 2, 2012 01:50 PM

Polypeptide:
Genbank accession no. NP_004954
Genbank version no. NP_004954.2 GI:222080083
Genbank record update date: Sep. 2, 2012 01:50 PM

### CROSS-REFERENCES

De Sauvage F. J., et al J. Biol. Chem. 266 (27), 17912-17918 (1991); Singh S., et al Biochem. Biophys. Res. Commun. 179 (3), 1455-1463 (1991)

Other Information:
Official Symbol: GUCY2C:
Other Aliases: DIAR6, GUC2C, MUCIL, STAR
Other Designations: GC-C; STA receptor; guanylyl cyclase C; hSTAR; heat-stable enterotoxin receptor; intestinal guanylate cyclase

### (62) Liv-1—SLC39A6 (Solute Carrier Family 39 (Zinc Transporter), Member 6)

Nucleotide:
Genbank accession no. U41060
Genbank version no. U41060.2 GI:12711792
Genbank record update date: Nov. 30, 2009 04:35 PM

Polypeptide:
Genbank accession no. AAA96258
Genbank version no. AAA96258.2 GI:12711793
Genbank record update date: Nov. 30, 2009 04:35 PM

### CROSS-REFERENCES

Taylor K M., et al Biochim Biophys Acta. 2003 Apr. 1; 1611(1-2):16-30

Other Information:
Official Symbol: SLC39A6
Other Aliases: LIV-1
Other Designations: LIV-1 protein, estrogen regulated; ZIP-6; estrogen-regulated protein LIV-1; solute carrier family 39 (metal ion transporter), member 6; solute carrier family 39 member 6; zinc transporter ZIP6; zrt- and Irt-like protein 6

### (63) 5T4, Trophoblast Glycoprotein, TPBG—TPBG (Trophoblast Glycoprotein)

Nucleotide:
Genbank accession no. AJ012159
Genbank version no. AJ012159.1 GI:3805946
Genbank record update date: Feb. 1, 2011 10:27 AM

Polypeptide:
Genbank accession no. CAA09930
Genbank version no. CAA09930.1 GI:3805947
Genbank record update date: Feb. 1, 2011 10:27 AM

### CROSS-REFERENCES

King K. W., et al Biochim. Biophys. Acta 1445 (3), 257-270 (1999)

Other Information:
Official Symbol: TPBG
Other Aliases: 5T4, 5T4AG, M6P1
Other Designations: 5T4 oncofetal antigen; 5T4 oncofetal trophoblast glycoprotein; 5T4 oncotrophoblast glycoprotein See WO2015/155345

### (64) CD56—NCMA1 (Neural Cell Adhesion Molecule 1)

Nucleotide:
Genbank accession no. NM_000615
Genbank version no. NM_000615.6 GI:336285433
Genbank record update date: Sep. 23, 2012 02:32 PM

Polypeptide:
Genbank accession no. NP_000606
Genbank version no. NP_000606.3 GI:94420689
Genbank record update date: Sep. 23, 2012 02:32 PM

### CROSS-REFERENCES

Dickson, G., et al, Cell 50 (7), 1119-1130 (1987)

Other Information:
Official Symbol: NCAM1
Other Aliases: CD56, MSK39, NCAM
Other Designations: antigen recognized by monoclonal antibody 5.1H11; neural cell adhesion molecule, NCAM

Antibodies:
Immunogen: HuN901 (Smith S V., et al Curr Opin Mol Ther. 2005 August; 7(4):394-401)
   for example, see humanized from murine N901 antibody. See FIGS. 1b and 1e of Roguska, M. A., et al. Proc Natl Acad Sci USA February 1994; 91:969-973.

### (65) CanAg (Tumor Associated Antigen CA242)

### CROSS-REFERENCES

Haglund C., et al Br J Cancer 60:845-851, 1989; Baeckstrom D., et al J Biol Chem 266:21537-21547, 1991

Antibodies:
huC242 (Tolcher A W et al., J Clin Oncol. 2003 Jan. 15; 21(2):211-22; Immunogen)
   for example, see US20080138898A1 SEQ ID NO: 1 and 2 (66) FOLR1 (Folate Receptor 1)

Nucleotide:
Genbank accession no. J05013
Genbank version no. J05013.1 GI:182417
Genbank record update date: Jun. 23, 2010 08:47 AM

Polypeptide:
Genbank accession no. AAA35823
Genbank version no. AAA35823.1 GI:182418
Genbank record update date: Jun. 23, 2010 08:47 AM

### CROSS-REFERENCES

Elwood P. C., et al J. Biol. Chem. 264 (25), 14893-14901 (1989)

Other Information:
Official Symbol: FOLR1
Other Aliases: FBP, FOLR
Other Designations: FR-alpha; KB cells FBP; adult folate-binding protein; folate binding protein; folate receptor alpha; folate receptor, adult; ovarian tumor-associated antigen MOv18

Antibodies:
M9346A—Whiteman K R., et al Cancer Res Apr. 15, 2012; 72(8 Supplement): 4628 (Immunogen)

### (67) GPNMB (Glycoprotein (Transmembrane) Nmb)

Nucleotide:
Genbank accession no. X76534
Genbank version no. X76534.1 GI:666042
Genbank record update date: Feb. 2, 2011 10:10 AM

Polypeptide:
Genbank accession no. CAA54044
Genbank version no. CAA54044.1 GI:666043
Genbank record update date: Feb. 2, 2011 10:10 AM

### CROSS-REFERENCES

Weterman M. A., et al Int. J. Cancer 60 (1), 73-81 (1995)

Other Information:
Official Symbol: GPNMB
Other Aliases: UNQ1725/PRO9925, HGFIN, NMB
Other Designations: glycoprotein NMB; glycoprotein nmb-like protein; osteoactivin; transmembrane glycoprotein HGFIN; transmembrane glycoprotein NMB

Antibodies:
Celldex Therapeutics: CR011 (Tse K F., et al Clin Cancer Res. 2006 Feb. 15; 12(4):1373-82)
   for example, see EP1827492B1 SEQ ID NO: 22, 24, 26, 31, 33 and 35

### (68) TIM-1—HAVCRI (Hepatitis A Virus Cellular Receptor 1)

Nucleotide:
Genbank accession no. AF043724
Genbank version no. AF043724.1 GI:2827453
Genbank record update date: Mar. 10, 2010 06:24 PM

Polypeptide:
Genbank accession no. AAC39862
Genbank version no. AAC39862.1 GI:2827454
Genbank record update date: Mar. 10, 2010 06:24 PM

### CROSS-REFERENCES

Feigelstock D., et al J. Virol. 72 (8), 6621-6628 (1998)

Other Information:
Official Symbol: HAVCR1
Other Aliases: HAVCR, HAVCR-1, KIM-1, KIM1, TIM, TIM-1, TIM1, TIMD-1, TIMD1
Other Designations: T cell immunoglobin domain and mucin domain protein 1; T-cell membrane protein 1; kidney injury molecule 1

### (69) RG-1/Prostate Tumor Target Mindin—Mindin/RG-1

### CROSS-REFERENCES

Parry R., et al Cancer Res. 2005 Sep. 15; 65(18):8397-405

### (70) B7-H4—VTCN1 (V-Set Domain Containing T Cell Activation Inhibitor 1

Nucleotide:
Genbank accession no. BX648021
Genbank version no. BX648021.1 GI:34367180
Genbank record update date: Feb. 2, 2011 08:40 AM

### CROSS-REFERENCES

Sica G L., et al Immunity. 2003 June; 18(6):849-61

Other Information:
Official Symbol: VTCN1
Other Aliases: RP11-229A19.4, B7-H4, B7H4, B7S1, B7X, B7h.5, PRO1291, VCTN1
Other Designations: B7 family member, H4; B7 superfamily member 1; T cell costimulatory molecule B7x; T-cell costimulatory molecule B7x; V-set domain-containing T-cell activation inhibitor 1; immune costimulatory protein B7-H4

### (71) PTK7 (PTK7 Protein Tyrosine Kinase 7)

Nucleotide:
Genbank accession no. AF447176
Genbank version no. AF447176.1 GI:17432420
Genbank record update date: Nov. 28, 2008 01:51 PM

Polypeptide:
Genbank accession no. AAL39062
Genbank version no. AAL39062.1 GI:17432421
Genbank record update date: Nov. 28, 2008 01:51 PM

### CROSS-REFERENCES

Park S. K., et al J. Biochem. 119 (2), 235-239 (1996)

Other Information:
Official Symbol: PTK7
Other Aliases: CCK-4, CCK4
Other Designations: colon carcinoma kinase 4; inactive tyrosine-protein kinase 7; pseudo tyrosine kinase receptor 7; tyrosine-protein kinase-like 7 (72) CD37 (CD37 molecule)

Nucleotide:
Genbank accession no. NM_001040031
Genbank version no. NM_001040031.1 GI:91807109
Genbank record update date: Jul. 29, 2012 02:08 PM

Polypeptide:
Genbank accession no. NP_001035120
Genbank version no. NP_001035120.1 GI:91807110
Genbank record update date: Jul. 29, 2012 02:08 PM

### CROSS-REFERENCES

Schwartz-Albiez R., et al J. Immunol. 140 (3), 905-914 (1988)

Other Information:
Official Symbol: CD37
Other Aliases: GP52-40, TSPAN26
Other Designations: CD37 antigen; cell differentiation antigen 37; leukocyte antigen CD37; leukocyte surface antigen CD37; tetraspanin-26; tspan-26

Antibodies:
Boehringer Ingelheim: mAb 37.1 (Heider K H., et al Blood. 2011 Oct. 13; 118(15):4159-68)
Trubion: CD37-SMIP (G28-1 scFv-Ig) ((Zhao X., et al Blood. 2007; 110: 2569-2577)
   for example, see US20110171208A1 SEQ ID NO: 253
Immunogen: K7153A (Deckert J., et al Cancer Res Apr. 15, 2012; 72(8 Supplement): 4625)

### (73) CD138-SDC1 (Syndecan 1)

Nucleotide:
Genbank accession no. AJ551176
Genbank version no. AJ551176.1 GI:29243141
Genbank record update date: Feb. 1, 2011 12:09 PM

Polypeptide:
Genbank accession no. CAD80245
Genbank version no. CAD80245.1 GI:29243142
Genbank record update date: Feb. 1, 2011 12:09 PM

### CROSS-REFERENCES

O'Connell F P., et al Am J Clin Pathol. 2004 February; 121(2):254-63

Other Information:
Official Symbol: SDC1
Other Aliases: CD138, SDC, SYND1, syndecan
Other Designations: CD138 antigen; heparan sulfate proteoglycan fibroblast growth factor receptor; syndecan proteoglycan 1; syndecan-1

Antibodies:
Biotest: chimerized MAb (nBT062)—(Jagannath S., et al Poster ASH #3060, 2010; WIPO Patent Application WO/2010/128087)
   for example, see US20090232810 SEQ ID NO: 1 and 2
Immunogen: B-B4 (Tassone P., et al Blood 104_3688-3696)
   for example, see US20090175863A1 SEQ ID NO: 1 and 2 (74) CD74 (CD74 molecule, major histocompatibility complex, class II invariant chain)

Nucleotide:
Genbank accession no. NM_004355
Genbank version no. NM_004355.1 GI:343403784
Genbank record update date: Sep. 23, 2012 02:30 PM

Polypeptide:
Genbank accession no. NP_004346
Genbank version no. NP_004346.1 GI:10835071
Genbank record update date: Sep. 23, 2012 02:30 PM

### CROSS-REFERENCES

Kudo, J., et al Nucleic Acids Res. 13 (24), 8827-8841 (1985)

Other Information:
Official Symbol: CD74
Other Aliases: DHLAG, HLADG, II, Ia-GAMMA
Other Designations: CD74 antigen (invariant polypeptide of major histocompatibility complex, class II antigen-associated); HLA class II histocompatibility antigen gamma chain; HLA-DR antigens-associated invariant chain; HLA-DR-gamma; Ia-associated invariant chain; MHC HLA-DR gamma chain; gamma chain of class II antigens; p33

Antibodies:
Immunomedics: hLL1 (Milatuzumab)—Berkova Z., et al Expert Opin Investig Drugs. 2010 January; 19(1):141-9)
   for example, see US20040115193 SEQ ID NOs: 19, 20, 21, 22, 23 and 24
Genmab: HuMax-CD74 (see website)

### (75) Claudins—CLs (Claudins)

### CROSS-REFERENCES

Offner S., et al Cancer Immunol Immunother. 2005 May; 54(5):431-45, Suzuki H., et al Ann N Y Acad Sci. 2012 July; 1258:65-70)

In humans, 24 members of the family have been described—see literature reference.

### (76) EGFR (Epidermal Growth Factor Receptor)

Nucleotide:
Genbank accession no. NM_005228
Genbank version no. NM_005228.3 GI:41927737
Genbank record update date: Sep. 30, 2012 01:47 PM

Polypeptide:
Genbank accession no. NP_005219
Genbank version no. NP_005219.2 GI:29725609
Genbank record update date: Sep. 30, 2012 01:47 PM

### CROSS-REFERENCES

Dhomen N S., et al Crit Rev Oncog. 2012; 17(1):31-50

Other Information:
Official Symbol: EGFR
Other Aliases: ERBB, ERBB1, HER1, PIG61, mENA
Other Designations: avian erythroblastic leukemia viral (v-erb-b) oncogene homolog; cell growth inhibiting protein 40; cell proliferation-inducing protein 61; proto-oncogene c-ErbB-1; receptor tyrosine-protein kinase erbB-1

Antibodies:
BMS: Cetuximab (Erbitux)—Broadbridge V T., et al Expert Rev Anticancer Ther. 2012 May; 12(5):555-65.
   for example, see U.S. Pat. No. 6,217,866—ATTC deposit No. 9764.
Amgen: Panitumumab (Vectibix)—Argiles G., et al Future Oncol. 2012 April; 8(4):373-89
   for example, see U.S. Pat. No. 6,235,883 SEQ ID NOs: 23-38.
Genmab: Zalutumumab—Rivera F., et al Expert Opin Biol Ther. 2009 May; 9(5):667-74.
YM Biosciences: Nimotuzumab—Ramakrishnan M S., et al MAbs. 2009 January-February; 1(1):41-8.
   for example, see U.S. Pat. No. 5,891,996 SEQ ID NOs: 27-34.
      (77) Her3 (ErbB3)—ERBB3 (v-erb-b2 Erythroblastic Leukemia Viral Oncogene Homolog 3 (Avian))

Nucleotide:
Genbank accession no. M34309
Genbank version no. M34309.1 GI:183990
Genbank record update date: Jun. 23, 2010 08:47 PM

Polypeptide:
Genbank accession no. AAA35979
Genbank version no. AAA35979.1 GI:306841
Genbank record update date: Jun. 23, 2010 08:47 PM

### CROSS-REFERENCES

Plowman, G. D., et al., Proc. Natl. Acad. Sci. U.S.A. 87 (13), 4905-4909 (1990)

Other Information:
Official Symbol: ERBB3
Other Aliases: ErbB-3, HER3, LCCS2, MDA-BF-1, c-erbB-3, c-erbB3, erbB3-S, p180-ErbB3, p45-sErbB3, p85-sErbB3
Other Designations: proto-oncogene-like protein c-ErbB-3; receptor tyrosine-protein kinase erbB-3; tyrosine kinase-type cell surface receptor HER3

Antibodies:
Merimack Pharma: MM-121 (Schoeberl B., et al Cancer Res. 2010 Mar. 15; 70(6):2485-2494)
   for example, see US2011028129 SEQ ID NOs: 1, 2, 3, 4, 5, 6, 7 and 8.
      (78) RON—MST1R (Macrophage Stimulating 1 Receptor (c-Met-Related Tyrosine Kinase))

Nucleotide:
Genbank accession no. X70040
Genbank version no. X70040.1 GI:36109
Genbank record update date: Feb. 2, 2011 10:17 PM

Polypeptide:
Genbank accession no. CCA49634
Genbank version no. CCA49634.1 GI:36110
Genbank record update date: Feb. 2, 2011 10:17 PM

### CROSS-REFERENCES

Ronsin C., et al Oncogene 8 (5), 1195-1202 (1993)

Other Information:
Official Symbol: MST1R
Other Aliases: CD136, CDw136, PTK8, RON
Other Designations: MSP receptor; MST1R variant RON30; MST1R variant RON62; PTK8 protein tyrosine kinase 8; RON variant E2E3; c-met-related tyrosine kinase; macrophage-stimulating protein receptor; p185-Ron; soluble RON variant 1; soluble RON variant 2; soluble RON variant 3; soluble RONvariant 4

### (79) EPHA2 (EPH Receptor A2)

Nucleotide:
Genbank accession no. BC037166
Genbank version no. BC037166.2 GI:33879863
Genbank record update date: Mar. 6, 2012 01:59 PM

Polypeptide:
Genbank accession no. AAH37166
Genbank version no. AAH37166.1 GI:22713539
Genbank record update date: Mar. 6, 2012 01:59 PM

### CROSS-REFERENCES

Strausberg R. L., et al Proc. Natl. Acad. Sci. U.S.A. 99 (26), 16899-16903 (2002)

Other Information:
Official Symbol: EPHA2
Other Aliases: ARCC2, CTPA, CTPP1, ECK
Other Designations: ephrin type-A receptor 2; epithelial cell receptor protein tyrosine kinase; soluble EPHA2 variant 1; tyrosine-protein kinase receptor ECK

Antibodies:
Medimmune: 1C1 (Lee J W., et al Clin Cancer Res. 2010 May 1; 16(9):2562-2570)
   for example, see US20090304721A1 FIGS. 7 and 8.

### (80) CD20—MS4A1 (Membrane-Spanning 4-Domains, Subfamily a, Member 1)

Nucleotide:
Genbank accession no. M27394
Genbank version no. M27394.1 GI:179307
Genbank record update date: Nov. 30, 2009 11:16 AM

Polypeptide:
Genbank accession no. AAA35581
Genbank version no. AAA35581.1 GI:179308
Genbank record update date: Nov. 30, 2009 11:16 AM

### CROSS-REFERENCES

Tedder T. F., et al Proc. Natl. Acad. Sci. U.S.A. 85 (1), 208-212 (1988)

Other Information:
Official Symbol: MS4A1
Other Aliases: B1, Bp35, CD20, CVIDS, LEU-16, MS4A2, S7
Other Designations: B-lymphocyte antigen CD20; B-lymphocyte cell-surface antigen B1; CD20 antigen; CD20 receptor; leukocyte surface antigen Leu-16

Antibodies:
Genentech/Roche: Rituximab—Abdulla N E., et al BioDrugs. 2012 Apr. 1; 26(2):71-82
   for example, see U.S. Pat. No. 5,736,137, ATCC deposit No. HB-69119.
GSK/Genmab: Ofatumumab—Nightingale G., et al Ann Pharmacother. 2011 October; 45(10):1248-55
   for example, see US20090169550A1 SEQ ID NOs: 2, 4 and 5.
Immunomedics: Veltuzumab—Goldenberg D M., et al Leuk Lymphoma. 2010 May; 51(5):747-55
   for example, see U.S. Pat. No. 7,919,273B2 SEQ ID NOs: 1, 2, 3, 4, 5 and 6.

### (81) Tenascin C—TNC (Tenascin C)

Nucleotide:
Genbank accession no. NM_002160
Genbank version no. NM_002160.3 GI:340745336
Genbank record update date: Sep. 23, 2012 02:33 PM

Polypeptide:
Genbank accession no. NP_002151
Genbank version no. NP_002151.2 GI:153946395
Genbank record update date: Sep. 23, 2012 02:33 PM

### CROSS-REFERENCES

Nies D. E., et al J. Biol. Chem. 266 (5), 2818-2823 (1991); Siri A., et al Nucleic Acids Res. 19 (3), 525-531 (1991)

Other Information:
Official Symbol: TNC
Other Aliases: 150-225, GMEM, GP, HXB, JI, TN, TN-C
Other Designations: GP 150-225; cytotactin; glioma-associated-extracellular matrix antigen; hexabrachion (tenascin); myotendinous antigen; neuronectin; tenascin; tenascin-C isoform 14/AD1/16

Antibodies:
Philogen: G11 (von Lukowicz T., et al J Nucl Med. 2007 April; 48(4):582-7) and F16 (Pedretti M., et al Lung Cancer. 2009 April; 64(1):28-33)
   for example, see U.S. Pat. No. 7,968,685 SEQ ID NOs: 29, 35, 45 and 47.

### (82) FAP (Fibroblast Activation Protein, Alpha)

Nucleotide:
Genbank accession no. U09278
Genbank version no. U09278.1 GI:1888315
Genbank record update date: Jun. 23, 2010 09:22 AM

Polypeptide:
Genbank accession no. AAB49652
Genbank version no. AAB49652.1 GI:1888316
Genbank record update date: Jun. 23, 2010 09:22 AM

### CROSS-REFERENCES

Scanlan, M. J., et al Proc. Natl. Acad. Sci. U.S.A. 91 (12), 5657-5661 (1994)

Other Information:
Official Symbol: FAP
Other Aliases: DPPIV, FAPA
Other Designations: 170 kDa melanoma membrane-bound gelatinase; integral membrane serine protease; seprase

### (83) DKK-1 (Dickkopf 1 Homolog (Xenopus Iaevis)

Nucleotide:
Genbank accession no. NM_012242
Genbank version no. NM_012242.2 GI:61676924
Genbank record update date: Sep. 30, 2012 01:48 PM

Polypeptide:
Genbank accession no. NP_036374
Genbank version no. NP_036374.1 GI:7110719
Genbank record update date: Sep. 30, 2012 01:48 PM

### CROSS-REFERENCES

Fedi P. et al J. Biol. Chem. 274 (27), 19465-19472 (1999)

Other Information:
Official Symbol: DKK1
Other Aliases: UNQ492/PRO1008, DKK-1, SK
Other Designations: dickkopf related protein-1; dickkopf-1 like; dickkopf-like protein 1; dickkopf-related protein 1; hDkk-1

Antibodies:
Novartis: BHQ880 (Fulciniti M., et al Blood. 2009 Jul. 9; 114(2):371-379)
   for example, see US20120052070A1 SEQ ID NOs: 100 and 108.

### (84) CD52 (CD52 Molecule)

Nucleotide:
Genbank accession no. NM_001803
Genbank version no. NM_001803.2 GI:68342029
Genbank record update date: Sep. 30, 2012 01:48 PM

Polypeptide:
Genbank accession no. NP_001794
Genbank version no. NP_001794.2 GI:68342030
Genbank record update date: Sep. 30, 2012 01:48 PM

### CROSS-REFERENCES

Xia M. Q., et al Eur. J. Immunol. 21 (7), 1677-1684 (1991)

Other Information:
Official Symbol: CD52
Other Aliases: CDW52
Other Designations: CAMPATH-1 antigen; CD52 antigen (CAMPATH-1 antigen); CDW52 antigen (CAMPATH-1 antigen); cambridge pathology 1 antigen; epididymal secretory protein E5; he5; human epididymis-specific protein 5

Antibodies:
Alemtuzumab (Campath)—Skoetz N., et al Cochrane Database Syst Rev. 2012 Feb. 15; 2:CD008078
   for example, see Drugbank Acc. No. DB00087 (BIOD00109, BTD00109) (85) CS1—SLAMF7 (SLAM family member 7)

Nucleotide:
Genbank accession no. NM_021181
Genbank version no. NM_021181.3 GI:1993571
Genbank record update date: Jun. 29, 2012 11:24 AM

Polypeptide:
Genbank accession no. NP_067004
Genbank version no. NP_067004.3 GI:19923572
Genbank record update date: Jun. 29, 2012 11:24 AM

### CROSS-REFERENCES

Boles K. S., et al Immunogenetics 52 (3-4), 302-307 (2001)

Other Information:
Official Symbol: SLAMF7
Other Aliases: UNQ576/PRO1138, 19A, CD319, CRACC, CS1
Other Designations: 19A24 protein; CD2 subset 1; CD2-like receptor activating cytotoxic cells; CD2-like receptor-activating cytotoxic cells; membrane protein FOAP-12; novel LY9 (lymphocyte antigen 9) like protein; protein 19A

Antibodies:
BMS: elotuzumab/HuLuc63 (Benson D M., et al J Clin Oncol. 2012 Jun. 1; 30(16):2013-2015)
   for example, see US20110206701 SEQ ID NOs: 9, 10, 11, 12, 13, 14, 15 and 16.

### (86) Endoglin—ENG (Endoglin)

Nucleotide:
Genbank accession no. AF035753
Genbank version no. AF035753.1 GI:3452260
Genbank record update date: Mar. 10, 2010 06:36 PM

Polypeptide:
Genbank accession no. AAC32802
Genbank version no. AAC32802.1 GI:3452261
Genbank record update date: Mar. 10, 2010 06:36 PM

### CROSS-REFERENCES

Rius C., et al Blood 92 (12), 4677-4690 (1998) Official Symbol: ENG

Other Information:
Other Aliases: RP11-228B15.2, CD105, END, HHT1, ORW, ORW1
Other Designations: CD105 antigen

### (87) Annexin A1—ANXA1 (Annexin A1)

Nucleotide:
Genbank accession no. X05908
Genbank version no. X05908.1 GI:34387
Genbank record update date: Feb. 2, 2011 10:02 AM

Polypeptide:
Genbank accession no. CCA29338
Genbank version no. CCA29338.1 GI:34388
Genbank record update date: Feb. 2, 2011 10:02 AM

### CROSS-REFERENCES

Wallner B. P., et al Nature 320 (6057), 77-81 (1986)

Other Information:
Official Symbol: ANXA1
Other Aliases: RP11-71A24.1, ANX1, LPC1
Other Designations: annexin I (lipocortin I); annexin-1; calpactin II; calpactin-2; chromobindin-9; lipocortin I; p35; phospholipase A2 inhibitory protein

### (88) V-CAM (CD106)-VCAM1 (Vascular Cell Adhesion Molecule 1)

Nucleotide:
Genbank accession no. M60335
Genbank version no. M60335.1 GI:340193
Genbank record update date: Jun. 23, 2010 08:56 AM

Polypeptide:
Genbank accession no. AAA61269
Genbank version no. AAA61269.1 GI:340194
Genbank record update date: Jun. 23, 2010 08:56 AM

### CROSS-REFERENCES

Hession C., et al J. Biol. Chem. 266 (11), 6682-6685 (1991)

Other Information:
Official Symbol VCAM1
Other Aliases: CD106, INCAM-100
Other Designations: CD106 antigen; vascular cell adhesion protein 1

In one embodiment, the ADC as defined anywhere herein comprises an antibody that is an anti-tumor antibody or antigen binding fragment thereof. In another embodiment, the antibody is selected from the group consisting of an anti-EphA2 antibody or antigen-binding fragment thereof, an anti-Her2 antibody or antigen-binding fragment thereof, an anti-GPC3 antibody or antigen-binding fragment thereof, an anti-ASCT2 antibody or antigen-binding fragment thereof and an anti-B7H4 antibody or antigen-binding fragment thereof.

The invention provides a pharmaceutical composition comprising the ADC of the invention. The invention provides a pharmaceutical composition comprising a IMT agent of the invention. The invention provides a pharmaceutical composition comprising a ADC and a IMT agent of the invention.

In one embodiment, the cancer as defined anywhere herein includes but is not limited to neoplasms and tumours (e.g., histocytoma, glioma, astrocyoma, osteoma), cancers (e.g. lung cancer, small cell lung cancer, gastrointestinal cancer, head and neck cancer, gastric cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), and leukemias. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin.

### FIGURE LEGENDS

FIG. 1: Vaccination model. (A): Schematic representation of vaccination/challenge mouse model experiment. (B): Percentage of tumor-free mice following challenge with CT26 cells treated with tubulysin, PBD, or radiation. Necrotic cells were used as a negative control (C): Number of tumor-free mice following challenge with necrotic CT26 cells, irradiated CT26 cells, tubulysin PBD,

FIG. 2: Rejection of new tumors in mice that achieved complete response with ADCs. (A): Table of complete responses in CT26 tumor-bearing mice that were treated with EphA2-1508 or EphA2-PBD. Mice were treated when tumor volumes were either 75 or 150 mm³. B): Mean tumor volume of cured mice that were re-challenged with CT26 cells on Day 138, along with growth of CT26 cells that were simultaneously implanted into naïve Balb/C animals. C): MCA205 tumor-bearing mice were dosed with EphA2 PBD. All mice achieved complete responses, and no tumors formed when mice were re-challenged on Day 43. Growth of MCA205 tumor in naïve animals is shown.

FIG. 3: AH1 ex-vivo stimulation assay from mice that achieved complete response. Splenic T cells from five mice that obtained complete response from EphA2-1508 and EphA2-PBD were assayed for IFN-gamma and TNF alpha production following ex-vivo stimulation by the AH1 peptide.

FIG. 4: ADCs had more activity in immunocompetent vs immunodeficient mice. Three syngeneic tumor models (CT26, 4T1, and MCA205) were evaluated for their anti-tumor response to EphA2-tubulysin (top panels) and EphA2-PBD (bottom panels) in immunodeficient (left panels) versus immunocompetent mice (right panels). (A): CT26 tumor model. (B): 4T1 tumor model. (C): MCA205 tumor model. ADCs were dosed when tumors were between 150-200 mm³.

FIG. 5: CD8 T cells are important for the efficacy of ADCs. Balb/C CT26 tumor-bearing mice were dosed with EphA2-Tub and EphA2-PBD simultaneously with a mixture of isotype control antibodies or a CD8-depleting antibody. The isoptype mix was also dosed separately.

FIG. 6: Synergistic effect of combination of ADCs with IMT. Balb/C mice bearing CT26 tumors were dosed with EphA2-PBD ADC or EphA2-tubulysin ADCalone and in combination with an anti-PD-L1 antibody or a mouse GITRL fusion protein. A) Untreated mice, B) EphA2-PBD, C) anti-PD-L1, D) EphA2-PBD+a-PD-L1, E) EphA2-tubulysin, F) GITRL FP, G) EphA2-tubulysin+GITRL FP, H) EphA2-PBD+a-PD-L1 (15 day post), and I) EphA2-tubulysin+GITRL FP (15 day post).

FIG. 7: Summary of combination studies with ADCs and anti-PD-1, anti-PD-L1, an OX40 ligand fusion protein, and GITRL ligand fusion protein

FIG. 8: CD8 T cells are important for ADC+anti-PD-L1 combination activity. Balb/C CT26 tumor-bearing mice were dosed with EphA2-Tub and EphA2-PBD simultaneously with α-PD-L1 and a mixture of isotype control antibodies or with α-PD-L1 and a CD8-depleting antibody. The isoptype mix was also dosed separately.

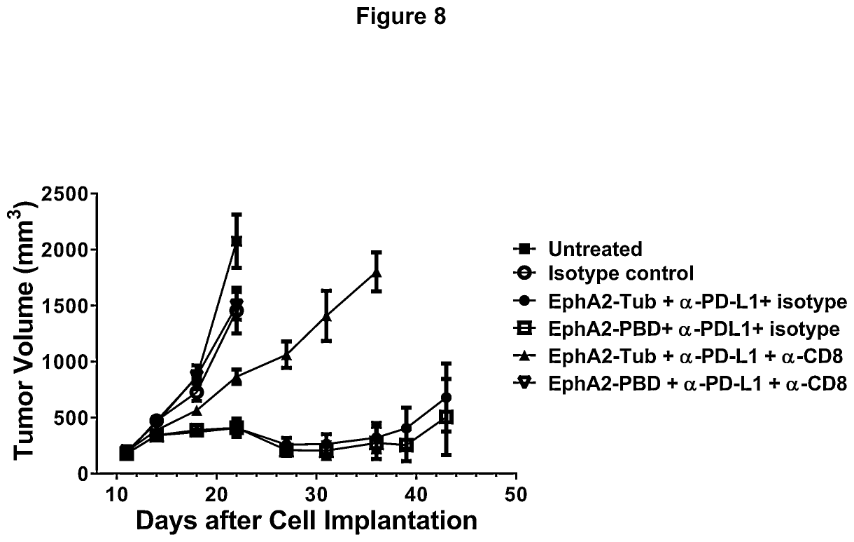

FIG. 9: Immunophenotyping of T-cell populations following ADC treatment. CT26 tumor-bearing mice were administered EphA2-PBD, EphA2-tubulysin alone or in combination with OX40 or PD-L1 antibodies. Spleen and tumor were harvested 5 days after drug administration. A), Percent of CD45 cells in tumor; B) Percent of CD45+CD8+ cells in tumor; C) Percent of CD45+CD8+CD69+ cells in tumor; D) Percent of CD45+CD8+PD-1+ cells in tumor; E) Percent of CD45+CD8+Ki67+ cells in tumor and F) Percent of CD45+CD4+Ki67+ cells in spleen

FIG. 10: Immunophenotyping of myeloid cell populations following ADC treatment. CT26 tumor-bearing mice were administered EphA2-PBD, EphA2-tubulysin alone or in combination with OX40 or PD-L1 antibodies. Spleen and tumor were harvested either 5 or 12 days after initial drug administration. A), Percent of CD45+CD86+ cells in tumor; B) Percent of CD45+CD80+ cells in tumor; C) Percent of CD45+F480+CD86+ cells in tumor; D) Mean fluorescence intensity (MFI) of CD86 in CD45+CD11c+ cells in tumor; E) Percent of CD45+GR−1^{hi}+CD11b^{hi}+CD86+ cells in tumor; F) MFI of CD86 in CD45+ cells in spleen; G) Percent of CD45+Gr−1^{int}+CD11b^{hi}+CD86+ cells in spleen; H) Percent of CD45+GR−1^{hi}+CD11b^{hi}CD86% in spleen; I)

FIG. 11: ADC+IMT combinations with different ADC in different syngeneic tumor models. A) Activity of IGF1R-PBD ADC (top right), α-PD-L1 (lower left) or the combination (lower right) in the CT26 model. CR number is out of 12 mice. B) Activity of EphA2-Tubuysin ADC (top right), OX40 ligand fusion protein (lower left) or the combination (lower right) in the MCA205 model. CR number is out of 12 mice C) Activity of EphA2-PBD ADC (top right), GITR ligand fusion protein (lower left) or the combination (lower right) in the Renca model. CR number is out of 10 mice.

### DETAILED DESCRIPTION OF THE INVENTION

The present invention is based upon the surprising and unexpected discovery that an ADC payload can have an effect on immune cells, specifically inducing tumor-specific immunological memory. The present invention is further based upon the surprising and unexpected discovery that the combination of an ADC and an IMT provides an enhanced effect to provide a potent combination therapy. PBDs and tubulysins represent two potent ADC payload classes that have an effect on immune cells. The present invention thus provides targeted immunomodulatory tumor-specific therapy.

The present invention provides a new combination therapy based on an ADC and an IMT agent.

### Antibody-Drug Conjugates (ADC)

The ADCs defined herein may be used to provide cytotoxic payloads to the target location (e.g., tumorigenic cells), promote intracellular accumulation of the drug within the tumor cells and subsequently induce apoptosis.

In one embodiment of the invention, the drug conjugated to the ADC is a pyrrolobenzodiazepine PBD. In another embodiment of the invention, the drug conjugated to the ADC is a tubulysin.

One example of a PBD dimer is SG2000 (SJG-136):

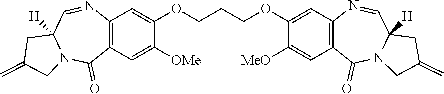

(Gregson, S., et al., J. Med. Chem., 44, 737-748 (2001); Alley, M. C., et al., Cancer Research, 64, 6700-6706 (2004); Hartley, J. A., et al., Cancer Research, 64, 6693-6699 (2004)). Other examples are known from the literature and will be apparent to the person skilled in the art.

Tubulysins for use in the present invention have the following general formula:

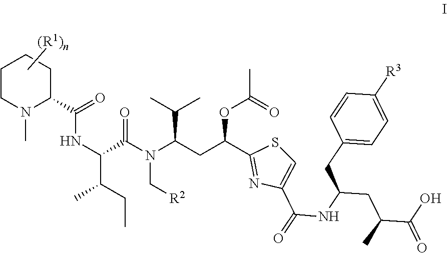

Further specific examples are disclosed in WO2015/157594, which is incorporated herein by reference.

PBDs are naturally occurring antibiotics that bind in the minor groove of DNA forming inter- and intra-strand cross-linked adducts (Hartley, 2011), incorporated herein by reference). Tubulysins are anti-mitotic agents that function to depolymerize microtubules (Li et al., 2016). These compounds have been shown to be extremely potent as ADC payloads (Saunders et al., 2015; Jeffrey et al., 2013, both of which are incorporated herein by reference).

The synthesis of PBD compounds is extensively discussed in the following references, which discussions are incorporated herein by reference:
a) WO 00/12508 (pages 14 to 30);
b) WO 2005/023814 (pages 3 to 10);
c) WO 2004/043963 (pages 28 to 29); and
d) WO 2005/085251 (pages 30 to 39).

The synthesis of tubulysin molecules for use in the present invention is discussed in WO 2015/157594, which is incorporated herein by reference.

In one embodiment of the invention, the antibody of the ADC is an anti-tumor antibody or antigen-binding fragment thereof. Potential antitumor targets include but are not limited to the tumor-associated antigens set forth above. This advantageously allows the ADC of the invention to be targeted to tumor cells. Such localization provides for relatively high concentrations of drug within the tumor. In order to identify suitable tumor-associated antigens to target with an ADC, two general approaches can be employed. An indication-dependent approach results from focusing on a particular cancer type and then conducting research that leads to selection of targets and then ADCs for that disease. Alternatively, indication-independent screens can be used to identify targets based on functional characteristics, such as internalization, rather than on a specific type of cancer. Once potential targets are identified, various cancers can be screened for expression of those targets to select a lead indication.

In a further embodiment of the invention, the antibody is an anti-EphA2 antibody or antigen-binding fragment thereof. In a still further embodiment of the invention, the antibody is 1C1 anti-EphA2 antibody (Jackson et al., 2008; US2011/028092 (SEQ ID NO:3)). EphA2 is abundantly expressed on several tumors.

In a further embodiment of the invention, the antibody is an anti-GPC3 antibody or antigen-binding fragment thereof.

In a further embodiment of the invention, the antibody is an anti-B7H4 antibody or antigen-binding fragment thereof.

In a further embodiment of the invention, the antibody is an anti-ASCT2 antibody or antigen-binding fragment thereof.

In a further embodiment of the invention, the antibody is an anti-Her2 antibody or antigen-binding fragment thereof.

In one embodiment of the invention, the antibody or antigen-binding fragment thereof is a monoclonal antibody. In another embodiment of the invention, the antibody or antigen-binding fragment thereof is a humanised antibody. In yet another embodiment of the invention, the antibody or antigen-binding fragment thereof is a human antibody.

In one embodiment of the invention, the antibody or antigen-binding fragment thereof is an IgA, IgD, IgE, IgG, IgM, IgG1 or IgG2 antibody or antigen-binding fragment thereof.

Antibodies or antigen-binding fragments thereof are not limited to a particular method of generation or production. Antibodies or antigen-binding fragments thereof can be prepared using a wide variety of techniques known in the art including hybridoma techniques, recombinant techniques, phage display technologies, transgenic animals (e.g., a XenoMouse®) or some combination thereof.

Antigen-binding fragments include Fab, Fv, scFv, dAb, Fd, Fab′, F(ab′)₂ or an isolated complementarity determining region (CDR) having sufficient framework to bind. A Fab fragment may be a monovalent fragment consisting of the VLC, VHC, CL and CHI domains. A F(ab′)₂ fragment may be a bivalent fragment comprising two Fab fragments linked by a disulfide bridge at the hinge region. A Fc fragment may consist of the VHC and CHI domains. A Fv fragment may consist of the VLC and VHC domains of a single arm of an antibody. A dAb fragment (Ward et al., 1989) incorporated herein by reference may consist of a VHC domain. An isolated CDR having sufficient framework to bind may be an antigen binding portion of a variable region.

An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see (Huston et al., 1988; Bird et al., 1988), both of which are incorporated herein by reference). These are obtained using conventional techniques known to those with skill in the art, and the portions are screened for utility in the same manner as are intact antibodies.

As would be well recognized by those skilled in the art, fragments can be obtained by molecular engineering or via chemical or enzymatic treatment (such as papain or pepsin) of an intact or complete antibody or antibody chain or by recombinant means. See (Paul, 1999) for a more detailed description of antibody fragments.

In one embodiment of the invention, the antibody or antigen-binding fragment thereof binds its target with high affinity. In one embodiment of the invention, the antibody or antigen-binding fragment thereof binds its target with a K_{D} of <50 nM as measured by BIAcore. In order to be effective the ADC not only needs to bind efficiently to the target on the surface of cells, but that binding needs to result in internalization of the ADC-antigen complex. Following internalization, the ADC needs to be metabolized in order to release the warhead and elicit cytotoxicity. In one embodiment of the invention the antibody or antigen-binding fragment internalizes following binding to its target antigen.

### Immunotherapy (IMT) Agent

In one embodiment, the IMT agent as defined anywhere herein is a checkpoint inhibitor. In another embodiment, the IMT agent as defined anywhere herein is an agonist of the tumor necrosis factor (TNF) receptor superfamily. In one embodiment, the IMT agent as defined anywhere herein is selected from the group consisting of: a PD1 inhibitor, a PD-L1 inhibitor, an OX40 agonist, and a GITRL agonist. In a further embodiment, the IMT agent as defined anywhere herein is selected from the group consisting of: an anti-PD1 antibody, an anti-PD-L1 antibody and an OX40 antibody, OX40 ligand fusion protein and a GITRL fusion protein.

### Pharmaceutical Preparations

The present invention provides pharmaceutical compositions comprising an ADC of the invention. The present invention also provides pharmaceutical compositions comprising an IMT agent of the invention. The present invention further provides pharmaceutical compositions comprising an ADC and an IMT agent of the invention. In one embodiment, the pharmaceutical composition comprises a pharmaceutically acceptable excipient.

Various pharmaceutically acceptable excipients, which include vehicles, adjuvants, and diluents, are readily available from numerous commercial sources. Certain non-limiting exemplary excipients include saline, buffered saline, dextrose, water, glycerol, ethanol, and combinations thereof. Moreover, an assortment of pharmaceutically acceptable auxiliary substances, such as pH adjusting and buffering agents, tonicity adjusting agents, stabilizers, wetting agents and the like, are also available. The pharmaceutical composition may contain suitable stabilisers or agents which increase the solubility of the compounds to allow for the preparation of highly concentrated solutions. Suitable pharmaceutically acceptable excipients may facilitate administration of the ADC or facilitate processing of the ADC into preparations that are pharmaceutically optimized for delivery to the site of action.

The pharmaceutical composition may take the form of an aqueous solution and may include physiologically compatible buffers such as Hank's solution, Ringer's solution, or physiologically-buffered saline. The pharmaceutical composition may additionally or alternatively contain substances which increase the viscosity of the suspension, such as sodium carboxymethyl cellulose, sorbitol, or dextran. The pharmaceutical composition may be prepared as appropriate oily injection suspensions. Suitable lipophilic solvents or vehicles include fatty oils such as sesame oil, or synthetic fatty acid esters, such as ethyl oleate or triglycerides, or liposomes.

Pharmaceutical compositions of the invention may be administered to a patient by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The pharmaceutical compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

In one embodiment, the pharmaceutical compositions of the invention are administered intravenously. In one embodiment, the pharmaceutical compositions of the invention are administered intraperitoneally. In one embodiment, the pharmaceutical compositions of the invention are administered intratumorally.

### Combination Therapy

The invention provides an ADC defined herein for use in cancer immunotherapy, wherein the use comprises administering to a patient the ADC in combination with an IMT agent. The invention also provides an IMT agent as defined anywhere herein for use in cancer immunotherapy, wherein the use comprises administering to a patient the IMT in combination with an ADC. The invention also provides an ADC defined herein and an IMT for use in cancer immunotherapy, wherein the use comprises administering to a patient the ADC in combination with the IMT agent.

The invention provides an ADC as defined anywhere herein for use in cancer immunotherapy, wherein the use comprises simultaneously, separately or sequentially administering to a patient the ADC in combination with an IMT agent. The invention also provides an IMT agent as defined anywhere herein for use in cancer immunotherapy, wherein the use comprises simultaneously, separately or sequentially administering to a patient the IMT agent in combination with an ADC. The invention also provides an ADC defined herein and an IMT agent for use in cancer immunotherapy, wherein the use comprises simultaneously, separately or sequentially administering to a patient the ADC in combination with the IMT agent.

The invention provides a cancer immunotherapy method, the method comprising administering to a patient an ADC defined herein and an IMT agent. The invention also provides a cancer immunotherapy method, the method comprising simultaneously, separately or sequentially administering to a patient an ADC defined herein and an IMT agent.

In use, the combination of the ADC defined herein and an IMT therapy provides an enhanced effect (e.g., additive or synergistic in nature). The combined results may be additive of the effects observed when each treatment (e.g., ADC and IMT therapy) is conducted separately. Although at least additive effects are generally desirable, any increased effect is beneficial.

In a preferred embodiment of the invention, in use, the combination of the ADC defined herein and an IMT therapy has a synergistic effect. Specifically, the efficacy of the cancer immunotherapy is increased. In addition, the anti-tumor response of the therapy is increased.

The combination treatment may be carried out in any way as deemed necessary or convenient by the person skilled in the art and for the purpose of this specification, no limitations with regard to the order, amount, repetition or relative amount of the components to be used in combination is contemplated.

The ADC defined herein and an IMT therapy may be administered to the patient simultaneously, either in a single composition or in separate compositions using the same or different administration routes.

Alternatively, the ADC and IMT may be in separate compositions and administered sequentially.

Preferably, administration of the ADC precedes or is simultaneous with the IMT therapy. Administration of the ADC may precede IMT therapy by at least 6, at least 12, at least 24, at least 48, at least 72 or at least 96 hours. Preferably, administration of the ADC is between 6 and 48 hours prior to IMT therapy. More preferably, administration of the ADC is between 12 and 24 hours prior to IMT therapy.

The combination therapy thus contemplates discontinuous administration or daily doses divided into several partial administrations. The time period between each delivery is such that the IMT therapy and ADC are able to exert a combined effect on the tumor. Administration may be effected by repeated administrations for a prolonged period of time. Administration can be concurrent or sequential, and can be effected in any order.

Administration of the ADC in combination with the IMT agent encompasses simultaneous and sequential administration as described above.

The dosage ranges for administration of the ADC defined herein and/or the IMT therapy are those to produce the desired therapeutic or prophylactic effect. It will be appreciated that the dosage range required depends on the precise nature of the ADC and/or the IMT therapy, the route of administration, the nature of the formulation, the age of the patient, the nature, extent or severity of the patient's condition, contraindications, if any, and the judgement of the attending physician. Variations in these dosage levels can be adjusted using standard empirical routines for optimisation. Determining the optimal dosage will generally involve the balancing of the level of therapeutic benefit against any risk or deleterious side effects. Sustained continuous release formulations of a subject pharmaceutical composition may be appropriate.

Administration to a patient of the ADC defined herein and/or the IMT therapy defined herein may be by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, intratumoral and intrathecal, or otherwise by implantation or inhalation. In one embodiment of the invention, the ADC defined herein is administered intravenously. In one embodiment of the invention, the ADC defined herein is administered intratumorally. In one embodiment of the invention, the IMT therapy defined herein is administered intravenously. In one embodiment of the invention, the IMT therapy defined herein is administered intraperitoneally. In one embodiment of the invention, the IMT therapy defined herein is administered intratumorally.

The appropriate doses of ADC defined herein and the IMT therapy defined herein may be around those already employed in clinical therapies wherein the IMT therapy is administered alone or in combination with other IMT therapies. Advantageously, in one embodiment, the ADC as defined anywhere herein is administered at a lower dosage compared to the dosage required to be therapeutically effective as a monotherapy. Advantageously, in another embodiment, the IMT agent as defined anywhere herein is administered at a lower dosage compared to the dosage required to be therapeutically effective as a monotherapy. In a further embodiment, the ADC as defined anywhere herein and the IMT agent as defined anywhere herein are both administered at lower dosages compared to the respective dosages for the IMT agent or the ADC required to be therapeutically effective as a monotherapy. Such sub-efficacious doses have reduced toxicity and thus improved safety profiles.

In one embodiment, the ADC as defined anywhere herein is administered at a dosage that is at least 90%, at least 80%, at least 70%, at least 60%, at least 50%, at least 40%, at least 30%, at least 20%, at least 10%, at least, 5%, at least 1% lower than the dosage required to be therapeutically effective as a monotherapy. In another embodiment, the IMT agent as defined anywhere herein is administered at a dosage that is at least 90%, at least 80%, at least 70%, at least 60%, at least 50%, at least 40%, at least 30%, at least 20%, at least 10%, at least, 5%, at least 1% lower than the dosage required to be therapeutically effective as a monotherapy. In a further embodiment, the ADC as defined anywhere herein and the IMT agent as defined anywhere herein are both administered at dosages that are at least 90%, at least 80%, at least 70%, at least 60%, at least 50%, at least 40%, at least 30%, at least 20%, at least 10%, at least, 5%, at least 1% lower than the respective dosages for the IMT agent or the ADC required to be therapeutically effective as a monotherapy. Suitable models for observing tumor growth are well known to the person skilled in the art and may vary depending on the indication being investigated.

In one embodiment of the invention, the patient is immunocompromised. In a further embodiment of the invention, the patient is immunocompetent. The immune response contributes to the anti-tumor activity observed on administration of an ADC as defined herein and thus a greater clinical response is observed in immunocompetent patients.

In one embodiment, the cancer immunotherapy comprises inducing tumor-specific immunological memory. In one embodiment, the cancer immunotherapy comprises inducing cancer-specific immunological memory. Such immunological memory may beneficially enhance any response to subsequent exposure to tumor antigen and prevent or reduce the recurrence of patient's tumor.

In one embodiment, the cancer immunotherapy comprises reducing tumor growth.

In one embodiment, the ADC or cancer immunotherapy comprises inducing immunogenic cell death.

In one embodiment, the cancer as defined anywhere herein includes but is not limited to neoplasms and tumours (e.g., histocytoma, glioma, astrocyoma, osteoma), cancers (e.g. lung cancer, small cell lung cancer, gastrointestinal cancer, head and neck cancer, gastric cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), and leukemias. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin.

In one embodiment, the cancer is colon cancer. In one embodiment, the cancer is lung cancer. In one embodiment, the cancer is breast cancer. In one embodiment, the cancer is renal cancer. The preferred cancer indication includes those that express the antigen that is targeted by the ADC.

### Therapeutic Uses of Novel ADCs

The present invention provides an ADC of the invention for use in medicine. The present invention also provides an ADC of the invention for use in cancer immunotherapy wherein the cancer immunotherapy comprises administering the ADC to a patient.

The invention also provides a method of preventing or treating disease, the method comprising administering to the patient an ADC of the invention. The present invention also provides a cancer immunotherapy method, the method comprising administering to a patient an ADC of the invention.

In one embodiment, said method of treating disease comprises administering a therapeutically effective amount of the ADC of the invention. In another embodiment, said method of preventing disease comprises administering a prophylactically effective amount of the ADC of the invention.

An ADC may be applied as a monotherapy or may involve, in addition to an ADC defined herein, administration of an IMT. The administration of an IMT may be in combination with, or as an adjunct to, or in conjunction with, an ADC defined herein and may be by way of simultaneous, sequential or separate dosing of the individual components of the treatment.

In use, an ADC defined herein may reduce tumor growth.

In use, an ADC defined herein may induce immunogenic cell death.

### Definitions

A “therapeutically effective amount” refers to the amount of the ADC, which when administered alone or in combination to a patient for treating disease, or at least one of the clinical symptoms of disease, is sufficient to affect such treatment of the disease, or symptom. The therapeutically effective amount can vary depending, for example, on the ADC and/or symptoms of the disease, the age, weight, and/or health of the patient to be treated, and the judgment of the prescribing physician. An appropriate therapeutically effective amount in any given instance may be ascertained by those skilled in the art or capable of determination by routine experimentation. A therapeutically effective amount is also one in which any toxic or detrimental effects of the ADC are outweighed by the beneficial effects.

Cancer immunotherapy either stimulates the activities of specific components of the immune system or counteract signals produced by cancer cells that suppress immune responses

### EXAMPLES

### Example 1: Materials and Methods

### Example 1.1: Antibodies, Reagents and Cell Lines

CT26, 4T1 and Renca cells were obtained from ATCC (Manassas, Va.). CT26 and 4T1 were maintained in RPMI media supplemented with 10% fetal bovine serum. Renca were maintained in EMEM supplemented with 10% fetal bovine serum. MCA205 cells were obtained from Agonox (Portland, Oreg.) and grown in RPMI supplemented with 10% fetal bovine serum. Cell lines were re-authenticated using STR-based DNA profiling and multiplex PCR (IDEXX Bioresearch, Columbia, Mo.). Anti-PD-1 (RMP1-14), anti-PD-L1 (10F.9G2), anti-CD4 (GK1.5), and anti-CD8 (53-6.7) were obtained from BioXCell (West Lebanon, N.H.). Mouse OX40 ligand fusion protein (OX40L FP), mouse GITR ligand fusion protein (GITRL FP), and isotype control antibodies were produced by MedImmune. To generate OX86 mlgG2a antibody, the OX86 hybridoma was purchased from Sigma. The Fc domain was then re-engineered to mouse IgG2a format by MedImmune.

### Example 1.2: Animal Studies

Cells were grown in monolayer culture, harvested by trypsinization, and implanted subcutaneously into mice. For the CT26 and Renca tumor models, 5×10⁵ cells were implanted in the right flank of 6-8 week old female BALB/c mice (Harlan, Indianapolis, Ind.) using a 26-gauge needle. For the MCA205 tumor model, 2.5×10⁵ cells were implanted in the right flank of 6-8 week old female C57BL/6 mice (Harlan, Indianapolis, Ind.) using a 26-gauge needle. For the 4T1 tumor model, 1×10⁵ cells were implanted in the right flank of 6-8 week old female BALB/c mice using a 26-gauge needle.

All antibodies and fusion proteins were dosed via intraperitoneal injection. Immunotherapeutic agent dosing in the CT26 model was as follows: anti-PD-L1 (30 mg/kg, 2×/week×4); anti-PD-1 (20 mg/kg; 2×/week×4); mouse OX40 ligand fusion protein (5 mg/kg; 2×/week×2); mouse GITR ligand fusion proten (5 mg/kg×6). Dosing in the MCA205 model was as follows: EphA2-tubulysin (3 mg/kg, single dose) and mouse OX40L FP (20 mg/kg, 2×/week×2). Dosing in the Renca model was as follows: EphA2-PBD (0.33 mg/kg, once a week×3), and mouse GITRL FP (1 mg/kg; 2×/week×6). ADCs were dosed by intravenous injection at 10 mL per kg of mouse body weight. At the beginning of treatment, mice were randomized by tumor volume and were dosed when tumors reached 100-200 mm³, with the exception of GITRL FP in the CT26 model which was dosed when tumors reached ˜300 mm³. For delayed dosing experiments, therapy was initiated 15 days after the ADC was administered using the same dose schedule as above.

The number of animals per group ranged from 10-12 animals per group as determined based on sample size calculations using nQuery software. Both tumor and body weight measurements were collected twice weekly and tumor volume was calculated using the equation (L×W²)/2, where L and W refer to the length and width dimensions, respectively. Error bars were calculated as the standard error of the mean. The general health of mice was monitored daily and all experiments were conducted in accordance to AAALAC and MedImmune IACUC guidelines for humane treatment and care of laboratory animals. Statistical analysis for synergy was determined as previously described (Rios-Doria et al., 2015).

For depletion studies, CD8 depleting antibody was administered (8 mg/kg) on Day 6, 10, 14 and 18 after tumor cell implantation. EphA2-tubulysin was dosed at 5 mg/kg and EphA2-PBD was dosed at 0.3 mg/kg on Day 11. Anti-PD-L1 was administered at 30 mg/kg on days 11, 14, 17, and 21.

### Example 1.3: AH-1 Stimulation Assay

Spleens from mice that achieved complete response from ADC treatment were processed, and cells were plated at 2×10⁶ cells per well in a 96-well plate. The cells were incubated with AH1 peptide (Anaspec #64798) at 10 μg/mL along with protein transport inhibitors (Ebioscience #00-4980-93) for four hours followed by evaluation by flow cytometry. The percentage of CD45⁺/CD8⁺ or CD45⁺/CD4⁺ that were also TNFα⁺ and/or IFNγ⁺ were then analyzed.

### Example 1.4: Pharmacodynamic Studies

CT26 cells (5×10⁵ cells/mouse) were implanted in the right flank of 6-8 week old BALB/c female mice. When tumors were ˜300 mm³, mice were dosed with EphA2-PBD (0.3 mg/kg), EphA2-tubulysin (5 mg/kg), anti-PDL1 (10F.9G2, 20 mg/kg) or OX40 monoclonal antibody (OX86, 5 mg/kg). ADCs were administered as one intravenous dose on Day 0. PD-L1 antibody was administered on Days 0, 4, 7 and 11 and OX40 antibody was administered on Days 0 and 4. On Day 5 and Day 12 following dosing, spleen and tumor were collected, processed and stained for flow cytometry. For the Renca model, dosing of EphA2-PBD, GITRL FP, or the combination started when tumors were ˜150 mm³. EphA2-PBD was dosed on D0, GITRL FP was dosed on Day 0 and 4, and tumors were harvested on Day 5. Red blood cells were lysed with ACK solution (Life Tech, Carlsbad, Calif.). Tumors were cut into 2 mm³ pieces and digested for 40 min. using a Miltenyi Tumor Dissociation Human kit (Miltenyi Biotec, San Diego, Calif.). Tissues were counted for viability and then plated at 1 million cells per well. Live Dead Blue (Life Tech, Carlsbad, Calif.) was stained at 1:1000 for 20 minutes at room temperature then washed and blocked using 4% mouse serum for 15 minutes at room temperature. Extracellular dyes were added then incubated at 4 degrees Celsius for 20 minutes in FACS buffer (PBS+2% FBS). Cells were then washed, fixed and permeabilized using a FOXP3 transcription kit (Ebioscience, San Diego, Calif.). Intracellular stains were applied for 30 minutes at room temperature. Cells were then washed and run on the LSRII or Fortessa flow cytometer (BD, San Diego). Antibodies used for flow cytometry staining include, CD8 (BD, Clone 5H10), CD11 b (BD, Clone M1/70), CD4 (Biolegend Clone RM4-5), CD11c (Biolegend Clone n418), CD86 (Biolegend, Clone GL-1), GR-1 (Biolegend, Clone RB6-8C5), MHC-II (Biolegend Clone M5/114.15.2), F4/80 (Biolegend, Clone BM8), CD69 (Biolegend, Clone H1.2F3), KI-67 (eBioscience, Clone SolA15), PD-1 (Ebioscience, Clone J43), FOXP3 (Ebioscience Clone FJK-16S), CD45 (eBioscience Clone 30-F11) and IFN-gamma (Clone XMG1.2). Data were analyzed using Flowjo software (Treestar, Ashland, Oreg.).

### Example 1.5: In Vivo Vaccination Studies

CT26 cells were treated with either 400 nM AZD9185 (tubulysin) or 8 nM SG3199 (PBD) for 24 hours so that the cells were committed to cell death (as assessed by lack of growth in re-plating experiments) but still >95% viable. Five-hundred thousand of these treated cells were inoculated into the left flank of BALB/c mice to test for their ability to vaccinate for subsequent right flank challenge. Controls included cells treated with 75 Gy radiation or taken through 3 freeze-thaw cycles (necrotic). Challenge on the right flank utilized 3×10⁶ untreated CT26 cells 7 days later.

### Example 1.6: ADC Production

The antibody to EphA2 has been previously described (Jackson et al., 2008). The PBD and tubulysin payloads were site-specifically conjugated to cysteines engineered into the Fc domain of the antibodies. Initially the antibodies are partially reduced by adding the antibody, at a 5-10 mg/mL concentration, to tris(2-carboxyethyl)phosphine (TCEP) dissolved in a pH-adjusted PBS EDTA buffer such that the resulting antibody/TCEP solution had a TCEP/mAb molar ratio of 40. The antibody/TCEP solution was incubated at 37° C. for 2.5-3 hours. The reduced antibody was buffer exchanged into conjugation reaction buffer (PBS, 1 mM EDTA, pH 7.2) using NAP columns for mg scale conjugations. Re-oxidation of the antibody interchain disulfide bonds was initiated by the addition of dehydrascorbic acid (dHAA) in DMSO solution to achieve a dHAA/mAb molar ratio of 20, and incubation at 20° C. for 3-4 hours.

The PBD or tubulysin payloads were prepared by dissolving these in DMSO to achieve a final concentration of 10 mM. For the conjugation reaction, DMSO is added slowly to the reduced antibody to a final concentration of 10% v/v, and subsequently the payload is added to the antibody to achieve a payload/mAb molar ratio of 12. The payload/mAb solution is incubated at 20° C. for 1 hour and the conjugation reaction is quenched by the addition of N-acetylcysteine (NAC) solution to achieve a NAC/mAb molar ratio of 48 and subsequent incubation at 20° C. for 15 minutes. The final ADC products were then buffer exchanged into either PBS or 25 mM histidine HCl, 200 mM sucrose and 0.02% w/v PS80, pH 6. The biochemical properties of the resulting ADC are characterized using size-exclusion chromatography high pressure liquid chromatography (SEC-HPLC) to determine purity and aggregation content, and by using hydrophobic interaction chromatography HPLC (HIC-HPLC) to confirm drug loading. Reduced glycosylated reverse-phase HPLC (RP-HPLC) and liquid chromatography-mass spectrometry (LC-MS) were conducted to determine the drug:antibody ratio (DAR) and specificity of site-specific conjugation. Typically these conjugation reactions produced ADCs with >98% monomer, with a conjugation efficiency of >90% correlating to a DAR>1.82.

### Example 2: Vaccination/Challenge Assays

Different anti-neoplastic compounds differ in their ability to orchestrate the release of immunogenic molecules from targeted dying cells. Immunogenic cell death involves induction of cell surface calreticulin as well as release of ATP and HMGB1 from dying cells which bind to CD91, P2RX7, and TLR4, respectively, on dendritic cells resulting in the potentiation of an adaptive immune response.

Since tubulysin and PBDs induce cell death through the fundamentally distinct mechanisms of microtubule inhibition and DNA crosslinking, respectively, it is understood that they might differ in their abilities to activate dendritic cells or boost immunity through other means.

### Example 2.1: Ability of PBD and Tubulysin to Vaccinate In Vivo

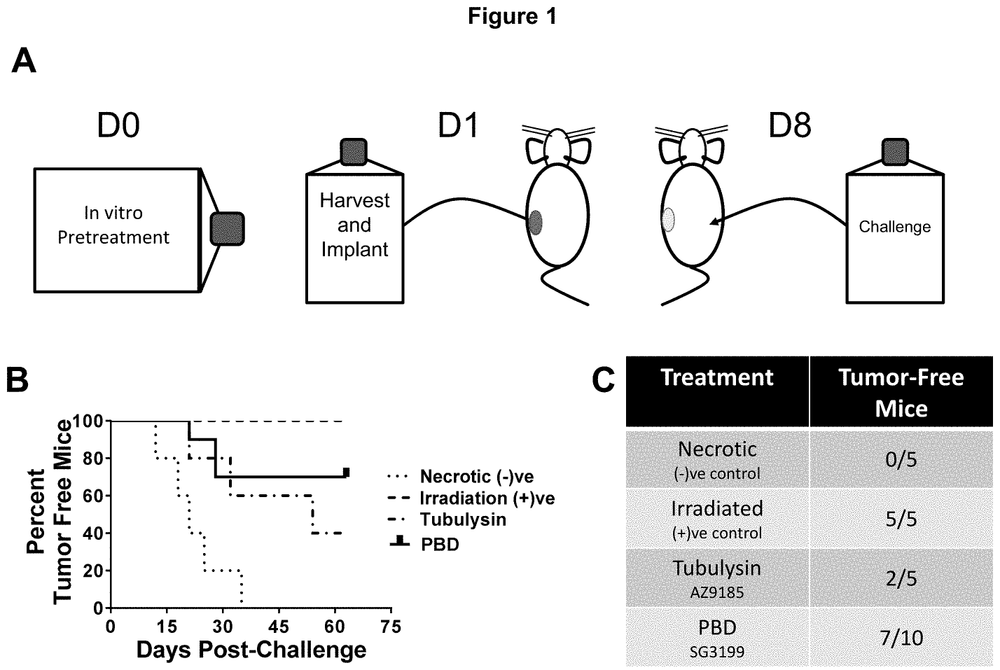

In order to test this, tubulysin and PBD were used in a vaccination/challenge mouse model. CT26 mouse colon cancer cells were treated with either tubulysin (1 uM) or PBD (10 nM) for a period of 24 hours (FIG. 1A). At this point in time, the cells were still viable but committed subsequent cell death.

These dying but not yet dead cells were implanted subcutaneously into the left flank of BALB/c mice to test if they could vaccinate against subsequent challenge with a tumor forming dose of untreated CT26 cells in the opposite flank 1 week later. Irradiated cells were included as a positive control and necrotic cells which were taken through a freeze thaw cycle were included as a negative control.

Necrotic cells provided did not vaccinate since by day 32 following CT26 challenge, there were no tumor free mice (FIGS. 1B and 1C). As expected, irradiated CT26 cells were highly immunogenic and protected all mice from tumors. Tubulysin and PBD treated cells protected 40 and 70% of mice, respectively, from tumor formation when assessed on day 65. These results demonstrate that tubulysin treated cells and PBD treated cells provided significant vaccination.

### Example 2.2: Ability of ADCs Comprising PBD or Tubulysin to Vaccinate In Vivo

Based on the results in Example 2.1, it was investigated whether treatment of tumor-bearing mice with ADCs conjugated with tubulysin or PBD payloads could also result in vaccination of mice.

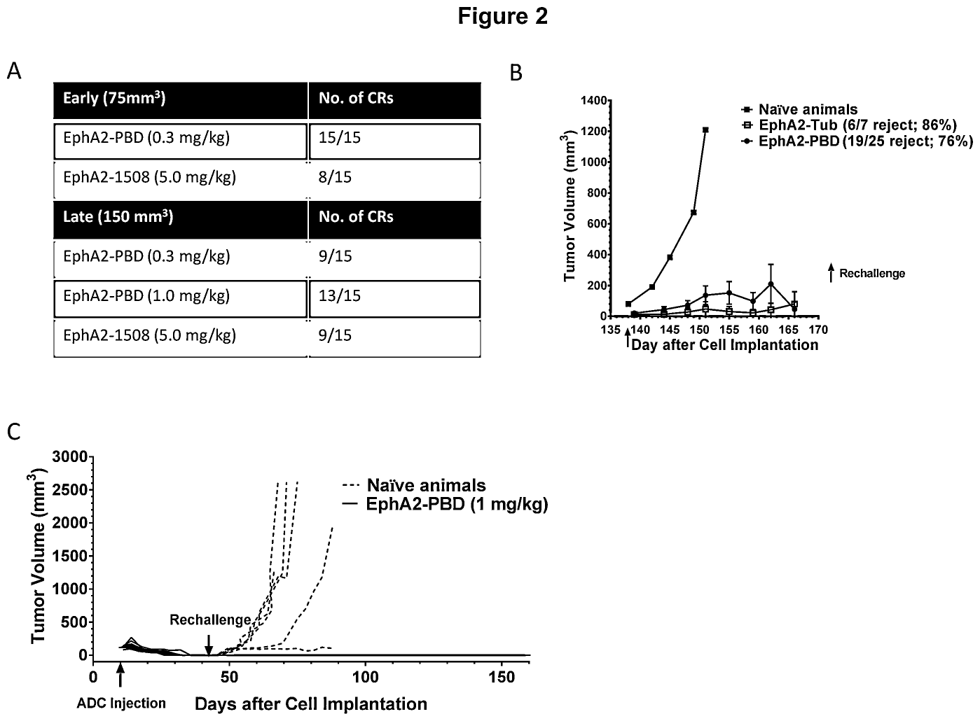

For these studies, an antibody targeting EphA2 was utilized and this was conjugated with a tubuylsin (AZ1508) or PBD (SG3315) payload. CT26 tumor-bearing BALB/c mice were treated with EphA2-tubulysin or EphA2-PBD at dose levels and frequency expected to result in a significant percentage of complete responses. Treatment of CT26 tumor-bearing mice when tumor volumes were either 75 mm³ or 150 mm³ produced a large percentage of complete responses (FIG. 2A). Following treatment and 90 days of observation, ˜57% of mice that received EphA2-tubulysin at 5 mg/kg had a complete response. In contrast, the majority of mice (82%) that received EphA2-PBD at 1 mg/kg had complete responses.

Subsequently, a subset of these tumor-free mice were re-challenged with CT26 cells, and 86% of mice cured with EphA2-tubulysin rejected the tumor cell challenge, while 76% of those cured with EphA2-PBD rejected the tumor cell challenge (FIG. 2B). CT26 cells grew readily in naïve BALB/c mice that were innoculated at the same time. These results demonstrate that cell-killing induced by these ADCs produced tumor-specific immunological memory. The ability of ADCs to induce memory was then evaluated in another syngeneic tumor model, MCA205. In this model, 10/10 animals achieved complete response when a single dose of EphA2-PBD was administered at 1 mg/kg. Following re-challenge with MCA205 cells at Day 43 (FIG. 2C), all mice remained tumor-free and no tumor growth was observed up to 160 days after initial cell implantation. In comparison, MCA205 tumors readily grew in naïve C57131/6 animals which were simultaneously implanted at the time of re-challenge. These data demonstrate that both EphA-Tub and EphA2-PBD provided significant vaccination to re-challenge in vivo.

### Example 2.3: Ability of ADCs to Induce Tumor-Specific Immunological Memory

To determine whether there were any functional changes in T cells from mice that achieved complete response following treatment with an ADC, splenic T cells from five mice that obtained complete response from either EphA2-tubulysin or EphA2-PBD were assayed for IFN-gamma and TNF alpha production following ex-vivo stimulation by the AH1 peptide, which is the immunodominant antigen of CT26 cells.

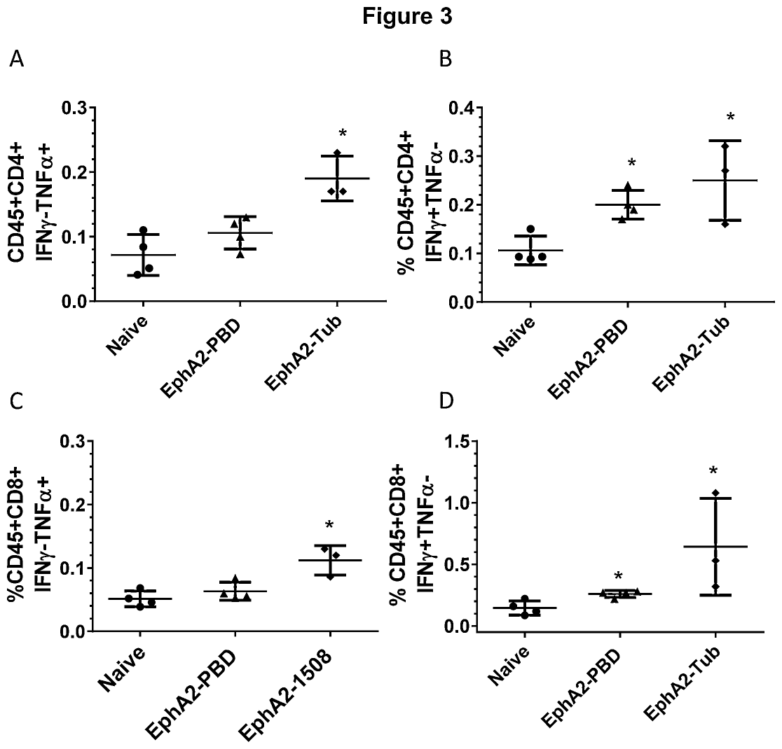

In this assay, CD4⁺ T cells from mice treated with EphA2-tubulysin produced TNF alpha upon stimulation with AH1 peptide (FIG. 3A), while CD4+ T cells from both EphA2-tubulysin and EphA2-PBD treated animals produced IFN-gamma (FIG. 3B). A similar pattern of expression was found on CD8⁺ T cells (FIG. 3C-D).

Taken together, these data demonstrate that these ADCs induced tumor-specific immunological memory and that these cells were functionally distinct from T cells from naïve mice. Also there appear to be payload-specific responses since only the EphA2-tubulysin induced TNF-alpha secreting T cells.

### Example 3: Role of the Immune System in the ADC-Induced Anti-Tumor Activity

Most antitumor studies evaluating ADCs have been performed in immunodeficient mice. Since the data in Example 2 suggests that T cells may play a role in anti-tumor activity, the anti-tumor activity of these EphA2 ADCs in T-cell deficient (nude) mice was evaluated compared to immunocompetent mice.

### Example 3.1: Anti-Tumor Activity in Immunocompetent Vs. Immunodeficient Mice

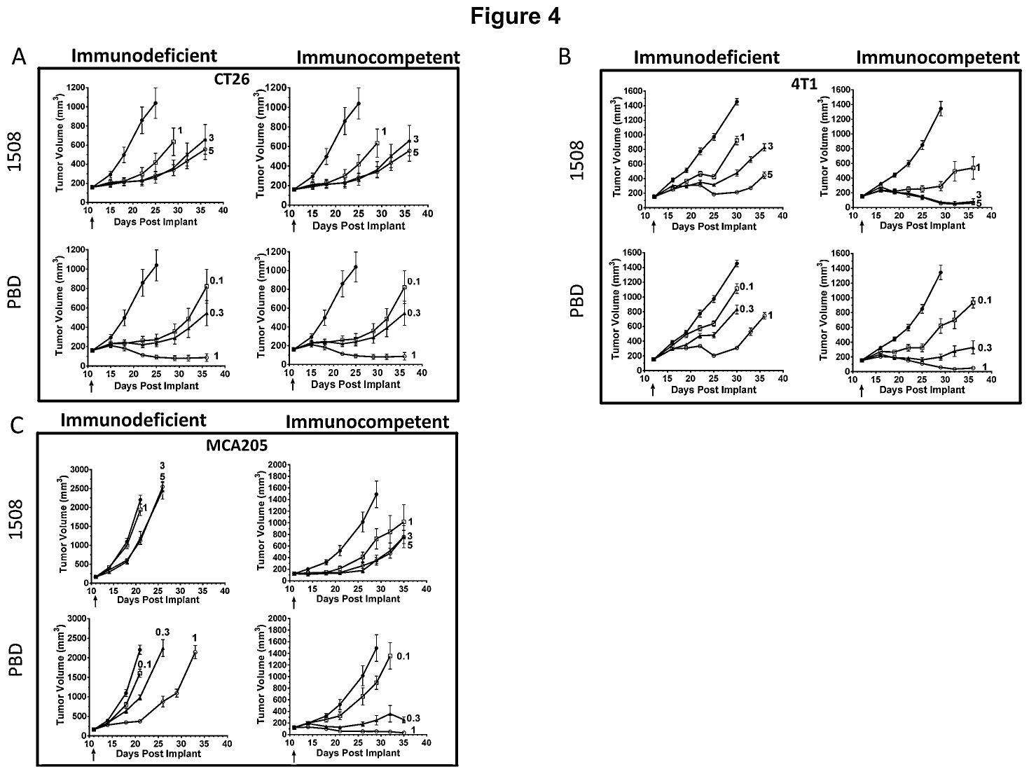

The anti-tumor activity of ADCs against three syngeneic tumor models (CT26, 4T1, and MCA205) grown in either immunocompetent or immunodeficient mice was evaluated (FIG. 4). In each tumor model, both EphA2-tubulysin (top panels) and EphA2-PBD (bottom panels) had more anti-tumor activity in immunocompetent mice (right panels) compared to immunodeficient mice (left panels).

These results demonstrate that a functional immune system is important for full activity of these ADCs, and suggest that T cells are important for the efficacy of ADCs in immunocompetent models.

### Example 3.2: Role of T Cells in Efficacy of ADCs

To test whether T cells are important for the efficacy of ADCs in immunocompetent models, CT26 tumor-bearing mice were treated with EphA2-tubuylsin or EphA2-PBD ADC along with a CD8 depleting antibody.

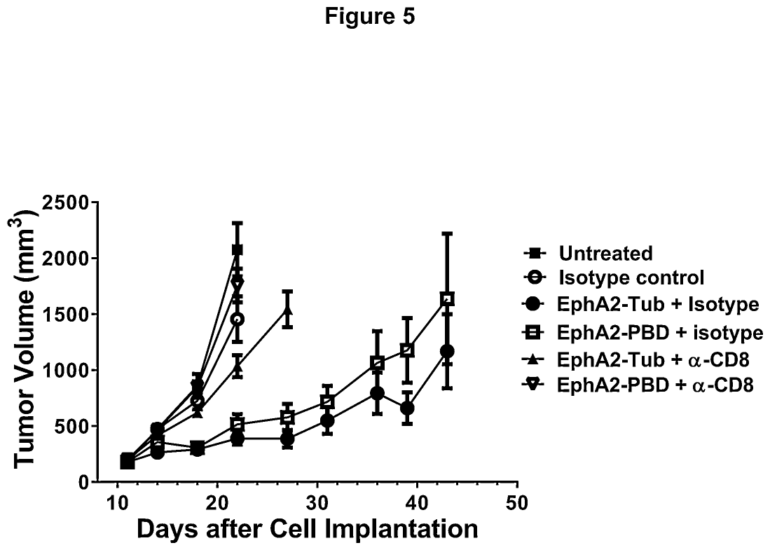

Strikingly, depletion of CD8⁺ T cells abrogated the efficacy of the ADCs alone (FIG. 5). There was no effect on ADC activity when dosing with an isotype control antibody. These results demonstrate that T cells are important for the efficacy of ADCs in immunocompetent models.

The above results demonstrate that an immune response is likely responsible for the ADC-induced increased anti-tumor activity.

### Example 4: Synergistic Effect of ADCs and Cancer Immunotherapies

The fact that cells killed with tubulysin or PBD could vaccinate mice against CT26 tumor re-challenge suggested that these warheads may induce immunogenic cell death. It was investigated whether ADCs carrying these warheads may be able to produce additive or potentially synergistic effects if combined with cancer immunotherapies.

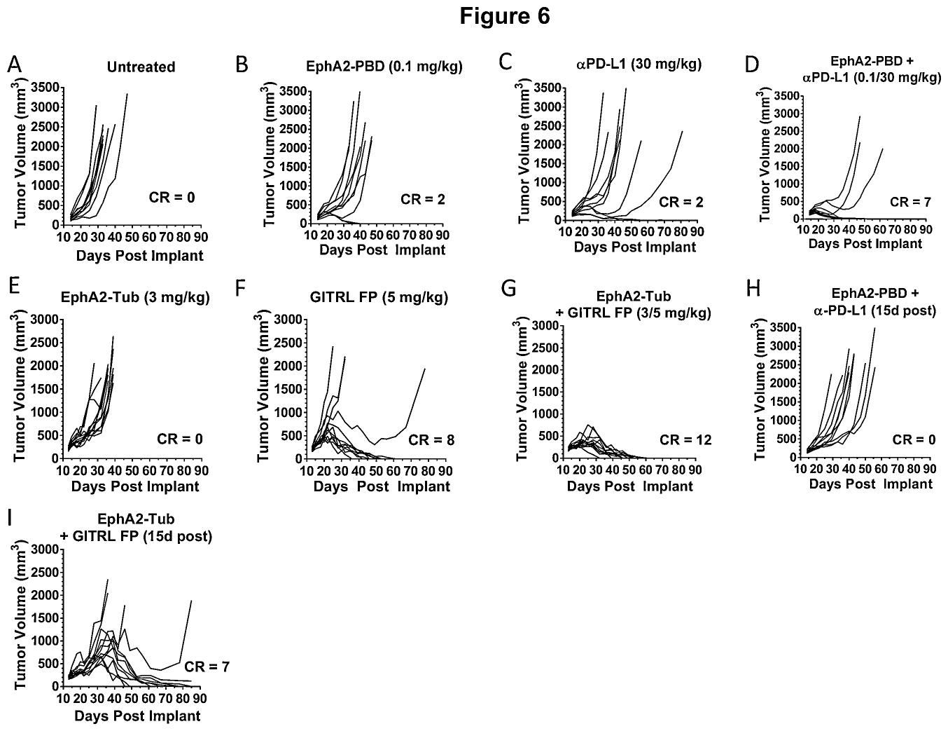

BALB/c mice bearing CT26 tumors were dosed with EphA2-ADCs alone or in combination with an anti-PD-L1 antibody (FIG. 6).

Treatment with either 0.1 mg/kg of EphA2-PBD or 30 mg/kg of anti-PD-L1 produced moderate anti-tumor activity with two complete responses (CRs), i.e. complete regressions, in each group of ten mice (FIGS. 6B and 6C). The combination of anti-PD-L1 and EphA2-PBD however produced a synergistic response with 7/10 CRs (FIG. 6D). It should be noted that 0.1 mg/kg of EphA2-PBD is a suboptimal dose of the ADC in the CT26 model (FIG. 4A).

Treatment of mice with EphA2-tubulysin ADC produced tumor growth delay but no CRs (FIG. 6E). Mice treated with a mouse GITR ligand fusion protein eliceted potent anti-tumor activity with 8/12 animals achieving CRs (FIG. 6F). However, the combination of EphA2-tubulysin ADC with GITRL FP produced a synergistic response with CRs observed in all (12/12) mice (FIG. 6G).

The aforementioned studies were performed through dosing the ADCs and immunotherapy simultaneously. When dosing of the immunotherapy was delayed, a much different outcome was observed. When anti-PD-L1 was dosed 15 days after EphA2-PBD ADC (FIG. 6H), no combination effect was observed. A much reduced combination effect was observed with GITRL FP was dosed 15 days after EphA2-tubulysin ADC (FIG. 6I). These data demonstrate that the schedule of dosing of ADCs and IMTs is important for activity.

Combination studies with these ADCs combined with either anti-PD-1 antibodies or an OX40 ligand fusion protein were also performed. Results obtained from these combination studies are shown in FIG. 6. These results demonstrate that the combination of these ADCs with these cancer immunotherapies produced synergistic anti-tumor responses compared to single agent activities.

These results demonstrate that combining ADCs with checkpoint inhibitors or agonists of the TNFR superfamily results in potent, enhanced anti-tumor effects in vivo.

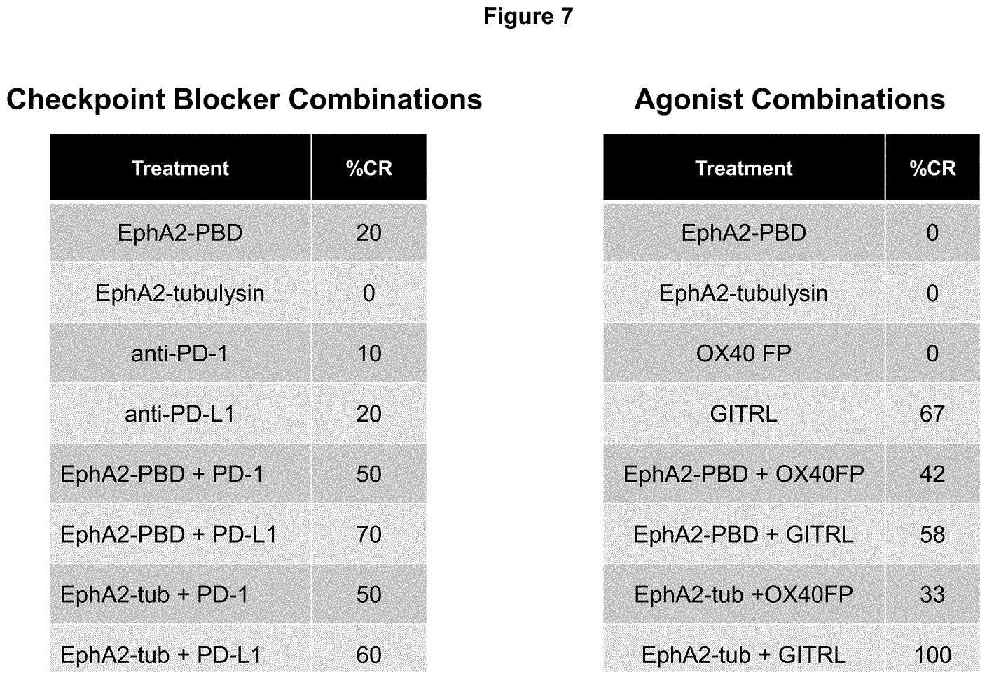

CD8 depletion also abrogated the activity of the ADCs in combination with anti-PD-L1 (FIG. 7 and see Example 3.2). These data demonstrate that CD8⁺ T cells play a role in the activity of ADCs in immunocompetent animals.

### Example 5: A Pharmacodynamics Study of ADCs

To determine the effects these ADCs were having on immune cells in vivo, a pharmacodynamics study was performed in the CT26 model.

CT26 tumor-bearing mice were administered EphA2-PBD or EphA2-tubulysin alone or in combination with PD-L1 or OX40 antibodies. Spleen and tumor were harvested 5 or 12 days after drug administration and investigated for changes in immune cell populations.

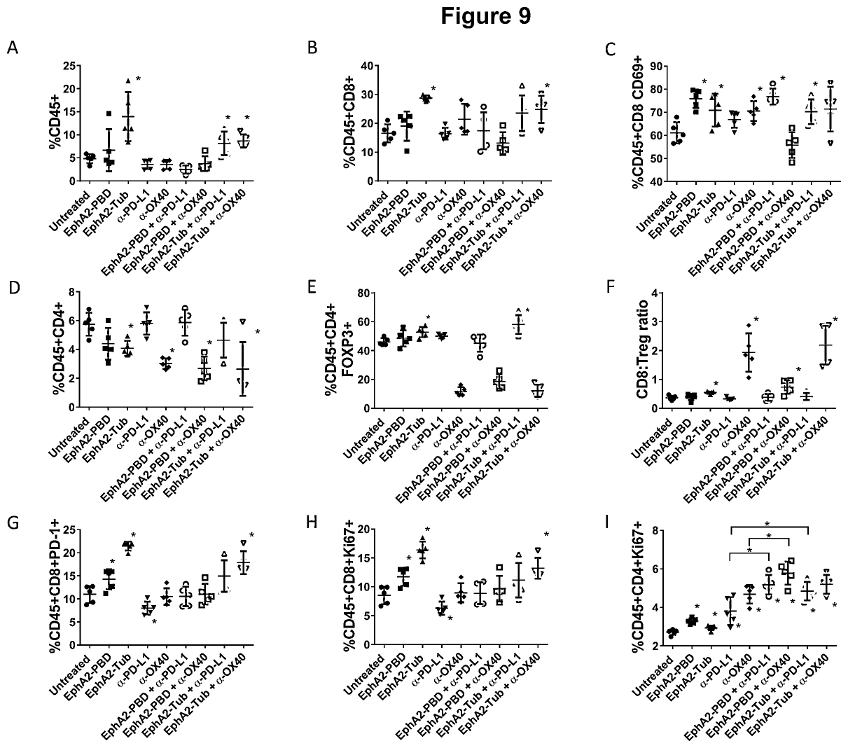

EphA2-tubulysin induced tumor infiltration of both CD45⁺ lymphocytes and CD45⁺CD8⁺ cytotoxic T lymphocytes (CTLs) (FIG. 9, A-B). While EphA2-PBD did not induce tumor-infiltration of these cell types, it did induce, along with EphA2-tubulysin, activation of pre-existing CD8⁺ cells in the tumor as identified by a CD8⁺CD69⁺ population (FIG. 9C). Activation of CD8+ cells was also observed with anti-OX40 treatment, and both EphA2-PBD and EphA2-tubulysin in combination with anti-PD-L1. EphA2-tubulysin, OX40 antibody alone and in combination with both ADCs decreased the number of total CD4⁺ T cells in the tumor (FIG. 9D). Within this subset, the number of FOXP3⁺ cells, a marker of regulatory T cells (T_{regs}), were slightly increased in the tumor following treatment with EphA2-tubulsysin alone or in combination with anti-PD-L1 (FIG. 9E). In contrast, the number of T_{regs} within the tumor was markedly decreased following treatment with either an OX40 antibody alone or in combination with either ADC (FIG. 9E). However, despite the slight increase in T_{regs} with EphA2-tubulysin, the CD8:Tre_{g} ratio in this group was increased overall compared to untreated tumors, as well as with OX40 antibody alone and in combination with both ADCs (FIG. 9F). Both ADCs induced PD-1 expression, as well as a higher percentage of CD8⁺ cells that were Ki67 positive, demonstrating increased CD8⁺ T cell proliferation (FIG. 9, G-H). PD-1 and Ki67 expression also increased on CD8⁺ cells following treatment with EphA-tubulysin in combination with OX40 antibodies. Both ADCs were also able to induce proliferation of CD4⁺ cells in the spleen as indicated by increased numbers of CD45⁺CD4⁺Ki67⁺ cells (FIG. 91). However, significantly larger increases in splenic CD4⁺ T cell proliferation were observed with EphA2-PBD in combination with anti-PD-L1 and OX40 and EphA2-tubulysin in combination with anti-PD-L1 compared to individual agents (FIG. 91). This finding highlights one potential mechanism by which ADC+IMT combination therapy led to significantly larger antitumor effects than single agent therapy.

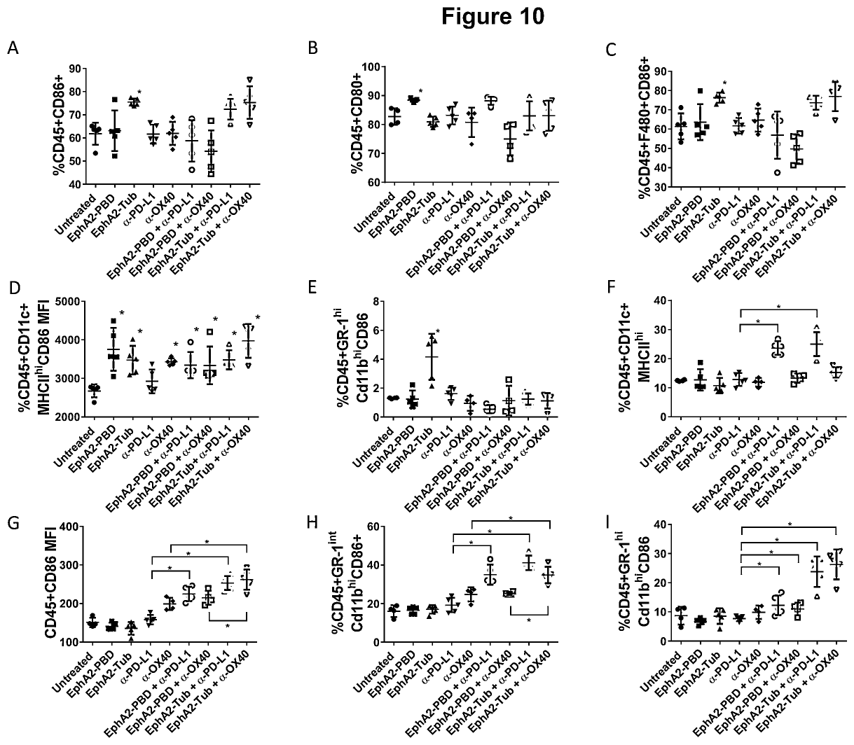

In examining changes to myeloid cells, ADCs were found to increase levels of CD86 in multiple cell populations. These results are shown in FIG. 10 as follows. EphA2-tubulysin alone and in combination with anti-PD-L1 or OX40 induced tumor-infiltration of CD45⁺CD86⁺ cells. These same groups also increased expression of CD86 on F480⁺ macrophages. As CD86 is a marker of increased antigen presentation, these results demonstrate that EphA2-tubulysin is able to directly induce tumor infiltration of cells that may possess this increased capability. Although EphA2-PBD did not induce tumor infiltration of CD45⁺CD86⁺ cells, EphA2-PBD did induce CD86 expression on CD45⁺CD11c⁺MHCII^{hi} mature dendritic cells either alone or in combination with anti-PD-L1 and OX40. Indeed, all treatment groups except anti-PD-L1 alone increased CD86 expression on mature dendritic cells. Interestingly, EphA2-tubulysin alone and in combination with anti-PD-L1, and EphA2-PBD in combination with anti-OX40 increased the percent of CD86⁺ granulocytic MDSCs in the tumor, suggesting an increased antigen-presentation phenotype on these cells.

The main phenotypic differences between combination and individual therapies were observed on myeloid cells in the spleen of CT26 tumor-bearing animals. EphA2-tubulysin or EphA2-PBD in combination with anti-PD-L1 increased the percent of CD45⁺CD11c⁺MHCII^{hi} mature dendritic cells in the spleen compared to anti-PD-L1 alone. Combining EphA2-tubulysin with anti-OX40, but not EphA2-PBD with OX40, had similar effects. Both EphA2-PBD and EphA2-tubulysin in combination with anti-PD-L1 increased CD86 expression on CD45⁺ lymphocytes compared to anti-PD-L1 alone. Interestingly, the combination of EphA2-tubulysin with anti-OX40, but not EphA2-PBD with anti-OX40, also increased CD86 expression on these cells. This phenotype represents a differentiating effect when OX40 antibodies are combined with tubulysin as compared to PBD-based ADCs. Additional examinations of myeloid populations in the spleen revealed modulation of CD86 expression on MDSCs. EphA2-PBD and EphA2-tubulysin in combination with anti-PD-L1 increased the percent of CD86⁺ monocytic MDSCs compared to individual therapies. The combination of EphA2-tubulysin with anti-OX40, but not EphA2-PBD combined with anti-OX40, also increased CD86⁺ monocytic MDSCs. EphA2-PBD and EphA2-tubulysin also increased the percent of CD86 expression on granulocytic MDSCs compared to anti-PD-L1 alone. The combination of EphA2-tubulysin and anti-OX40, but not EphA2-PBD and anti-OX40, increased the percent of CD86+ granulocytic MDSCs in the spleen. Strikingly, the combination of EphA2-tubulysin with anti-PD-L1 and anti-OX40 induced significantly higher infiltration of these cells as compared to the EphA2-PBD combinations. These results demonstrate that ADCs with either PBD or tubulysin payloads induce immunophenotypic changes within both the tumor and spleen.

The results demonstrate that the two different ADC payloads induce immunomodulatory effects, which has previously been unreported. Although PBD and tubulysin have distinct cytotoxic mechanisms of action, in an immunocompetent background, both payloads were able to induce an immunogenic cell death that manifests when ADCs conjugated with these payloads were dosed in combination with immunotherapy. The finding that both payloads were able to induce antigen-specific immunological memory also supports this. Specifically, the results demonstrate that the effects of these ADC payloads on immune cells reveal a role for these payloads in dendritic cell activation, leading to potent combination with immunotherapies, which is CD8⁺ T cell-dependent.

Taken together these data suggest that combinatorial therapies with ADCs and immunotherapy discussed herein could provide an increased clinical response.

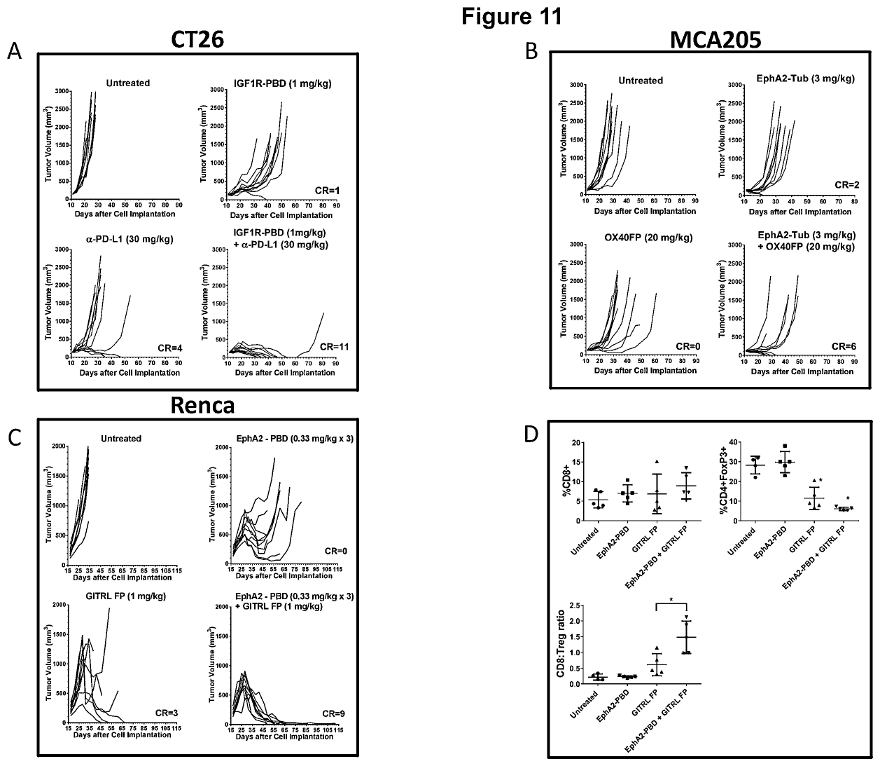

All of the data presented thus far has been using the CT26 model, which is known to be sensitive to many immunotherapies, and using ADCs targeting a single tumor-associated antigen, EphA2. To confirm that the effects of the ADC payloads observed with the EphA2 targeting ADCs are not model-dependent and can be applied to ADCs against other targets, we set out to determine whether ADCs can combine with immunotherapy in a different syngeneic tumor model, and also whether synergy could be observed using ADCs targeting a different tumor-associated antigen. CT26 cells overexpress the IGF1R receptor, therefore an antibody recognizing mouse IGF1R was conjugated with the PBD payload. IGF1R-PBD given as a single administration of 1 mg/kg in established CT26 tumors yielded one CR (FIG. 11A, top right). Anti-PD-L1 treatment resulted in 4 CRs (FIG. 11A, bottom left). However, the combination results in synergistic effects with CRs observed in 11/12 mice. These results demonstrate that synergy with ADCs and immunotherapy could be observed with a different ADC.

The EphA2 ADCs were then evaluated in the MCA205 tumor model as this model also overexpresses the EphA2 receptor. MCA205 tumor-bearing mice were dosed with EphA2-tubulysin (FIG. 11B, top right), an OX40 ligand fusion protein (FIG. 9B, bottom left) and the combination (FIG. 9B, bottom right). EphA2-tubulysin treatment resulted in 2CRs, OX40 FP treatment resulted in 0 CRs but the combination treatment resulted in 6/12 CRs. These results demonstrate that synergistic antitumor effects resulting from combing ADCs and immunotherapy may also be observed in diverse tumor models representing different patient tumor phenotypes.

EphA2-PBD was then evaluated in the Renca tumor model. As agonist antibodies to GITR have been shown to deplete Tre_{g}s in mouse models, and the Renca model was previously shown to be dependent on Tre_{g}s for growth (25), we examined the effect of combining a Tre_{g}-depleting mouse GITR ligand fusion protein (mGITRL FP) with EphA2-PBD ADC. Renca tumor-bearing mice were treated with EphA2-PBD (FIG. 11C, top right), mGITRL FP (FIG. 12C, bottom left), or the combination (FIG. 12C, bottom right). Treatment of mice with EphA2-PBD resulted in no CRs, whereas mGITRL FP induced 3 CRs. Strikingly, the combination of EphA2-PBD and mGITRL FP produced CRs in 8 of 10 mice. These data demonstrated yet a third tumor model in which an ADC bearing a PBD payload demonstrated synergy with an immunotherapy.

### Example 6: Differences Between ADC Payloads

While ADCs with either tubulysin or PBD payloads were able to synergize with immunotherapy, most of the differences between these were observed when the immune microenvironment was examined following treatment of mice with these ADCs.

EphA2 ADCs conjugated with the tubulysin payload consistently induced CD86 expression in a number of myeloid compartments, including on dendritic cells, macrophages and MDSCs. The same effect was only observed with the EphA2-PBD in some instances,

Taken together, these findings support the idea that these payloads are inducing immunogenic cell death, and at least for dendritic cells, CD86 upregulation may indicate an increase in antigen-presentation function for these cells.

### REFERENCES

Bird, R. E., K. D. Hardman, J. W. Jacobson, S. Johnson, B. M. Kaufman, S. M. Lee, T. Lee, S. H. Pope, G. S. Riordan, and M. Whitlow. 1988. Single-chain antigen-binding proteins. Science. 242:423-426.
Callahan, M. K., and J. D. Wolchok. 2013. At the bedside: CTLA-4- and PD-1-blocking antibodies in cancer immunotherapy. J. Leukoc. Biol. 94:41-53.
Galluzzi, L., L. Senovilla, L. Zitvogel, and G. Kroemer. 2012. The secret ally: immunostimulation by anticancer drugs. Nat. Rev. Drug Discov. 11:215-233.
Gerber, H. P., P. Sapra, F. Loganzo, and C. May. 2016. Combining antibody-drug conjugates and immune-mediated cancer therapy: What to expect? Biochem. Pharmacol. 102:1-6.
Hartley, J. A. 2011. The development of pyrrolobenzodiazepines as antitumour agents. Expert Opin. Investig. Drugs. 20:733-744.
Hu-Lieskovan, S., S. Mok, B. Homet Moreno, J. Tsoi, L. Robert, L. Goedert, E. M. Pinheiro, R. C. Koya, T. G. Graeber, B. Comin-Anduix, and A. Ribas. 2015. Improved antitumor activity of immunotherapy with BRAF and MEK inhibitors in BRAF(V600E) melanoma. Sci. Transl. Med. 7:279ra41.
Huston, J. S., D. Levinson, M. Mudgett-Hunter, M. S. Tai, J. Novotny, M. N. Margolies, R. J. Ridge, R. E. Bruccoleri, E. Haber, and R. Crea. 1988. Protein engineering of antibody binding sites: recovery of specific activity in an anti-digoxin single-chain Fv analogue produced in Escherichia coli. Proc. Natl. Acad. Sci. U.S.A. 85:5879-5883.
Jackson, D., J. Gooya, S. Mao, K. Kinneer, L. Xu, M. Camara, C. Fazenbaker, R. Fleming, S. Swamynathan, D. Meyer, P. D. Senter, C. Gao, H. Wu, M. Kinch, S. Coats, P. A. Kiener, and D. A. Tice. 2008. A human antibody-drug conjugate targeting EphA2 inhibits tumor growth in vivo. Cancer Res. 68:9367-9374.
Jeffrey, S. C., P. J. Burke, R. P. Lyon, D. W. Meyer, D. Sussman, M. Anderson, J. H. Hunter, C. I. Leiske, J. B. Miyamoto, N. D. Nicholas, N. M. Okeley, R. J. Sanderson, I. J. Stone, W. Zeng, S. J. Gregson, L. Masterson, A. C. Tiberghien, P. W. Howard, D. E. Thurston, C. L. Law, and P. D. Senter. 2013. A potent anti-CD70 antibody-drug conjugate combining a dimeric pyrrolobenzodiazepine drug with site-specific conjugation technology. Bioconjug. Chem. 24:1256-1263.
Khalil, D. N., E. L. Smith, R. J. Brentjens, and J. D. Wolchok. 2016. The future of cancer treatment: immunomodulation, CARs and combination immunotherapy. Nat. Rev. Clin. Oncol.
Kroemer, G., L. Galluzzi, O. Kepp, and L. Zitvogel. 2013. Immunogenic cell death in cancer therapy. Annu. Rev. Immunol. 31:51-72.
Larkin, J., V. Chiarion-Sileni, R. Gonzalez, J. J. Grob, C. L. Cowey, C. D. Lao, D. Schadendorf, R. Dummer, M. Smylie, P. Rutkowski, P. F. Ferrucci, A. Hill, J. Wagstaff, M. S. Carlino, J. B. Haanen, M. Maio, I. Marquez-Rodas, G. A. McArthur, P. A. Ascierto, G. V. Long, M. K. Callahan, M. A. Postow, K. Grossmann, M. Sznol, B. Dreno, L. Bastholt, A. Yang, L. M. Rollin, C. Horak, F. S. Hodi, and J. D. Wolchok. 2015. Combined Nivolumab and Ipilimumab or Monotherapy in Untreated Melanoma. N. Engl. J. Med. 373:23-34.
Li, J. Y., S. R. Perry, V. Muniz-Medina, X. Wang, L. K. Wetzel, M. C. Rebelatto, M. J. Hinrichs, B. Z. Bezabeh, R. L. Fleming, N. Dimasi, H. Feng, D. Toader, A. Q. Yuan, L. Xu, J. Lin, C. Gao, H. Wu, R. Dixit, J. K. Osbourn, and S. R. Coats. 2016. A Biparatopic HER2-Targeting Antibody-Drug Conjugate Induces Tumor Regression in Primary Models Refractory to or Ineligible for HER2-Targeted Therapy. Cancer. Cell. 29:117-129.
Liu, L., P. A. Mayes, S. Eastman, H. Shi, S. Yadavilli, T. Zhang, J. Yang, L. Seestaller-Wehr, S. Y. Zhang, C. Hopson, L. Tsvetkov, J. Jing, S. Zhang, J. Smothers, and A. Hoos. 2015. The BRAF and MEK Inhibitors Dabrafenib and Trametinib: Effects on Immune Function and in Combination with Immunomodulatory Antibodies Targeting PD-1, PD-L1, and CTLA-4. Clin. Cancer Res. 21:1639-1651.
Martin, K., P. Muller, J. Schreiner, S. S. Prince, D. Lardinois, V. A. Heinzelmann-Schwarz, D. S. Thommen, and A. Zippelius. 2014. The microtubule-depolymerizing agent ansamitocin P3 programs dendritic cells toward enhanced anti-tumor immunity. Cancer Immunol. Immunother. 63:925-938.
Muller, P., M. Kreuzaler, T. Khan, D. S. Thommen, K. Martin, K. Glatz, S. Savic, N. Harbeck, U. Nitz, O. Gluz, M. von Bergwelt-Baildon, H. Kreipe, S. Reddy, M. Christgen, and A. Zippelius. 2015. Trastuzumab emtansine (T-DM1) renders HER2+ breast cancer highly susceptible to CTLA-4/PD-1 blockade. Sci. Transl. Med. 7:315ra188.
Muller, P., K. Martin, S. Theurich, J. Schreiner, S. Savic, G. Terszowski, D. Lardinois, V. A. Heinzelmann-Schwarz, M. Schlaak, H. M. Kvasnicka, G. Spagnoli, S. Dirnhofer, D. E. Speiser, M. von Bergwelt-Baildon, and A. Zippelius. 2014a. Microtubule-depolymerizing agents used in antibody-drug conjugates induce antitumor immunity by stimulation of dendritic cells. Cancer. Immunol. Res. 2:741-755.
Muller, P., K. Martin, S. Theurich, M. von Bergwelt-Baildon, and A. Zippelius. 2014b. Cancer chemotherapy agents target intratumoral dendritic cells to potentiate antitumor immunity. Oncoimmunology. 3: e954460.
Obeid, M., A. Tesniere, F. Ghiringhelli, G. M. Fimia, L. Apetoh, J. L. Perfettini, M. Castedo, G. Mignot, T. Panaretakis, N. Casares, D. Metivier, N. Larochette, P. van Endert, F. Ciccosanti, M. Piacentini, L. Zitvogel, and G. Kroemer. 2007. Calreticulin exposure dictates the immunogenicity of cancer cell death. Nat. Med. 13:54-61.
Paul, W. E. 1999. Fundamental Immunology, Fourth Edition. 1589 pp.
Rios-Doria, J., N. Durham, L. Wetzel, R. Rothstein, J. Chesebrough, N. Holoweckyj, W. Zhao, C. C. Leow, and R. Hollingsworth. 2015. Doxil synergizes with cancer immunotherapies to enhance antitumor responses in syngeneic mouse models. Neoplasia. 17:661-670.
Saunders, L. R., A. J. Bankovich, W. C. Anderson, M. A. Aujay, S. Bheddah, K. Black, R. Desai, P. A. Escarpe, J. Hempl, A. Laysang, D. Liu, J. Lopez-Molina, M. Milton, A. Park, M. A. Pysz, H. Shao, B. Slingerland, M. Torgov, S. A. Williams, O. Foord, P. Howard, J. Jassem, A. Badzio, P. Czapiewski, D. H. Harpole, A. Dowlati, P. P. Massion, W. D. Travis, M. C. Pietanza, J. T. Poirier, C. M. Rudin, R. A. Stull, and S. J. Dylla. 2015. A DLL3-targeted antibody-drug conjugate eradicates high-grade pulmonary neuroendocrine tumor-initiating cells in vivo. Sci. Transl. Med. 7:302ra136.
Sievers, E. L., and P. D. Senter. 2013. Antibody-drug conjugates in cancer therapy. Annu. Rev. Med. 64:15-29.
Smyth, M. J., S. F. Ngiow, A. Ribas, and M. W. Teng. 2016. Combination cancer immunotherapies tailored to the tumour microenvironment. Nat. Rev. Clin. Oncol. 13:143-158.
Tesniere, A., F. Schlemmer, V. Boige, O. Kepp, I. Martins, F. Ghiringhelli, L. Aymeric, M. Michaud, L. Apetoh, L. Barault, J. Mendiboure, J. P. Pignon, V. Jooste, P. van Endert, M. Ducreux, L. Zitvogel, F. Piard, and G. Kroemer. 2010. Immunogenic death of colon cancer cells treated with oxaliplatin. Oncogene. 29:482-491.
Vanneman, M., and G. Dranoff. 2012. Combining immunotherapy and targeted therapies in cancer treatment. Nat. Rev. Cancer. 12:237-251.
Ward, E. S., D. Gussow, A. D. Griffiths, P. T. Jones, and G. Winter. 1989. Binding activities of a repertoire of single immunoglobulin variable domains secreted from Escherichia coli. Nature. 341:544-546.
Wolchok, J. D., H. Kluger, M. K. Callahan, M. A. Postow, N. A. Rizvi, A. M. Lesokhin, N. H. Segal, C. E. Ariyan, R. A. Gordon, K. Reed, M. M. Burke, A. Caldwell, S. A. Kronenberg, B. U. Agunwamba, X. Zhang, I. Lowy, H. D. Inzunza, W. Feely, C. E. Horak, Q. Hong, A. J. Korman, J. M. Wigginton, A. Gupta, and M. Sznol. 2013. Nivolumab plus ipilimumab in advanced melanoma. N. Engl. J. Med. 369:122-133.

1-15. (canceled)

16. A cancer immunotherapy method, the method comprising administering an antibody-drug conjugate (ADC) and an immune-mediated therapy (IMT) agent, wherein the method comprises administering to a patient the ADC in combination with the IMT agent, and
administering the ADC at a lower dosage compared to the dosage required to be therapeutically effective as a monotherapy; or
administering the IMT agent at a lower dosage compared to the dosage required to be therapeutically effective as a monotherapy; or
administering both the IMT agent and the ADC at lower dosages compared to the respective dosages for the IMT agent or the ADC required to be therapeutically effective as a monotherapy.

17. The method according to claim 16, wherein the ADC is administered at a dosage that is at least 90%, at least 80%, at least 70%, at least 60%, at least 50%, at least 40%, at least 30%, at least 20%, at least 10%, at least, 5%, at least 1% lower than the dosage required to be therapeutically effective as a monotherapy.

18. The method according to claim 16, wherein the IMT agent is administered at a dosage that is at least 90%, at least 80%, at least 70%, at least 60%, at least 50%, at least 40%, at least 30%, at least 20%, at least 10%, at least, 5%, at least 1% lower than the dosage required to be therapeutically effective as a monotherapy.

19. The method according to claim 16, the method comprising simultaneously, separately or sequentially administering to a patient an ADC and an IMT agent.

20. The method according to claim 16, wherein the drug conjugated to the ADC is a PBD or a tubulysin.

21. The method according to claim 16, wherein the ADC is administered intravenously or intratumorally.

22. The method according to claim 16, wherein the IMT agent is administered intravenously, intraperitoneally or intratumorally.

23. The method according to claim 16, wherein the IMT agent is a checkpoint inhibitor.

24. The method according to claim 16, wherein the IMT agent is an agonist of the tumor necrosis factor (TNF) receptor superfamily.

25. The method according to claim 16, wherein the IMT agent is selected from the group consisting of: a PD1 inhibitor, a PD-L1 inhibitor, an OX40 agonist, and a GITRL agonist.

26. The method according to claim 25, wherein the IMT agent is selected from the group consisting of: an anti-PD1 antibody, an anti-PD-L1 antibody and an anti-OX40 antibody, OX40 ligand fusion protein and a GITRL fusion protein.

27. The method according claim 16, wherein the ADC comprises an antibody that is an anti-tumor antibody or antigen binding fragment thereof.

28. The method according to claim 27, wherein the antibody is selected from the group consisting of an anti-EphA2 antibody or antigen-binding fragment thereof, an anti-Her2 antibody or antigen-binding fragment thereof, an anti-GPC3 antibody or antigen-binding fragment thereof, an anti-ASCT2 antibody or antigen-binding fragment thereof and an anti-B7H4 antibody or antigen-binding fragment thereof.

29. (canceled)
